(19) United States
(12) Patent Application Publication
(10) Pub. No.: US 2023/0062612 A1
(43) Pub. Date: Mar. 2, 2023

(54) SAFETY SWITCHES FOR REGULATION OF GENE EXPRESSION

(71) Applicant: Sana Biotechnology, Inc., Seattle, WA (US)

(72) Inventors: Ryan CLARKE, Seattle, WA (US); Greg HOFFMAN, Seattle, WA (US); Andrew MAY, San Francisco, CA (US); Eleonore THAM, San Francisco, CA (US)

(21) Appl. No.: 17/793,008

(22) PCT Filed: Jan. 15, 2021
(86) PCT No.: PCT/US21/13735

§ 371 Date: Jul. 14, 2022

Related U.S. Application Data
(60) Provisional application No. 62/962,764, filed on Jan. 17, 2020.
(60) Provisional application No. 62/962,739, filed on Jan. 17, 2020.
(60) Provisional application No. 62/962,730, filed on Jan. 17, 2020.

(51) Int. Cl. A61K 35/28; C12N 15/63; C12N 5/074; C12N 15/117; C12N 15/90; C12N 15/86; C12N 15/62; A61K 35/34; A61K 35/407; A61K 35/22; A61K 35/39; A61K 35/30; A61K 35/17; A61K 35/44

(52) U.S. Cl. CPC A61K 35/28; C12N 15/635; C12N 5/0696; C12N 15/117; C12N 15/907; C12N 15/86; C12N 15/625; A61K 35/34; A61K 35/407; A61K 35/22; A61K 35/39; A61K 35/30; A61K 35/17; A61K 35/44; C12N 2310/17; C12N 2310/531; C12N 2740/15043

(57) ABSTRACT

Disclosed herein are cells including pluripotent stem cells that conditionally express an immunosuppressive factor and related methods of their use and generation. In some embodiments, the cells disclosed do not express MHC I and MHC II human leukocyte antigens, and in some cases, also do not express one or more TCR complexes. In some embodiments, hypoimmunogenicity of the cells is controlled by activation of a controllable expression system upon contacting the cells with a specific factor or agent.

### CROSS-REFERENCE

This application claims priority under 35 U.S.C. § 119(e) to U.S. Provisional Application Nos. 62/962,730 filed Jan. 17, 2020, 62/962,739 filed Jan. 17, 2020, and 62/962,764 filed Jan. 17, 2020, the disclosures of which are herein incorporated by reference in their entirety.

### INCORPORATION BY REFERENCE OF SEQUENCE LISTING

The present application is being filed along with a Sequence Listing in electronic format. The Sequence Listing is provided as a file entitled SANA006WO1SeqList.txt, created Jan. 14, 2021, which is 33,948 bytes in size. The information in the electronic format of the Sequence Listing is incorporated by reference in its entirety.

### BACKGROUND

Degenerative diseases pose a disproportionate threat to human health. Often age-related, these diseases result in the progressive deterioration of affected tissues and organs and, ultimately, disability and death of the affected subject. The promise of regenerative medicine is to replace diseased or missing cells with new healthy cells. Over the past five years, a new paradigm for regenerative medicine has emerged—the use of human pluripotent stem cells (hPSCs) to generate any adult cell type for transplantation into patients. In principle, hPSC-based cell therapies have the potential to treat most if not all degenerative illnesses, however the success of such therapies may be limited by a subject's immune response.

Strategies that have been considered to overcome the immune rejection include HLA-matching (e.g., identical twin or umbilical cord banking), the administration of immunosuppressive drugs to the subject, blocking antibodies, bone marrow suppression/mixed chimerism, HLA-matched stem cell repositories, and autologous stem cell therapy.

There remains a need for novel approaches, compositions and methods for overcoming immune rejection associated with cell therapies.

### BRIEF SUMMARY

In one aspect, provided herein is a method for controlling the immunogenicity of an engineered cell, the method comprising: (a) obtaining an isolated cell: (b) introducing into the isolated cell (i) a nucleic acid comprising an inducible RNA polymerase promoter operably linked to an shRNA sequence targeting an immunosuppressive factor and (ii) a nucleic acid comprising a promoter operably linked to a transactivator element corresponding to the inducible RNA polymerase promoter to produce an engineered cell; and (c) exposing the engineered cell to an exogenous factor to activate the transactivator element, thereby controlling the immunogenicity of the cell.

In one aspect, provided herein is a method for controlling the immunogenicity of an engineered cell, the method comprising: (a) obtaining an isolated cell: (b) introducing into the isolated cell a nucleic acid comprising (i) a sequence encoding an inducible degron element operably linked to an immunosuppressive factor or (ii) a sequence encoding an immunosuppressive factor operably linked to an inducible degron element to produce an engineered cell; and (c) exposing the engineered cell to an exogenous factor to activate the inducible degron element, thereby controlling the immunogenicity of the engineered cell.

In another aspect, provided herein is a method for controlling immunogenicity of an engineered cell comprising: (a) obtaining an isolated cell; (b) introducing into the isolated cell: (i) a first construct comprising from 5′ end to 3′ end: a first promoter and an immunosuppressive factor gene; (ii) a second construct comprising from 5′ end to 3′ end: a second promoter and a nucleic acid sequence encoding Cas9 or a variant thereof; and (ii) a third construct comprising from 5′ end to 3′ end: an inducible RNA polymerase promoter, a guide RNA (gRNA) sequence targeting the immunosuppressive factor, a third promoter, and a transactivator element corresponding to the inducible RNA polymerase promoter; and (c) exposing the engineered cell to an exogenous factor to activate the transactivator element, thereby controlling the immunogenicity of the engineered cell.

In yet another aspect, provided herein is a method for controlling the immunogenicity of an engineered cell, the method comprising: (a) obtaining an isolated cell; (b) introducing into the isolated cell (i) a nucleic acid comprising an inducible RNA polymerase promoter operably linked to an immune signaling factor gene and (ii) a nucleic acid comprising a promoter operably linked to a transactivator element corresponding to the inducible RNA polymerase promoter to produce an engineered cell; and (c) exposing the engineered cell to an exogenous factor to activate the transactivator element, thereby controlling the immunogenicity of the engineered cell.

In some embodiments, the method further comprises administering the engineered cell to a subject prior to step (c).

In some embodiments, the step (b) of any of the methods comprises introducing into the isolated cell a single nucleic acid construct comprising (i) the inducible RNA polymerase promoter operably linked the shRNA sequence targeting the immunosuppressive factor and (ii) the promoter operably linked to the transactivator element. In some embodiments, construct comprises from 5′ end to 3′ end: the inducible RNA polymerase promoter; the shRNA sequence; the promoter; and the transactivator element.

In some embodiments, the step (b) comprises introducing into the isolated cell a single nucleic acid construct comprising (i) the inducible RNA polymerase promoter operably linked the immune signaling factor gene and (ii) the promoter operably linked to the transactivator element.

In some embodiments, the construct comprises from 5′ end to 3′ end: the inducible RNA polymerase promoter, the immune signaling factor gene, the promoter, and the transactivator element.

In some embodiments, the isolated cell is engineered to exogenously express the immunosuppressive factor. In some embodiments, the isolated cell overexpresses the immunosuppressive factor in the absence of the exogenous factor that activates the transactivator element.

In some embodiments, the inducible RNA polymerase promoter is a U6Tet promoter. In some embodiments, the inducible RNA polymerase promoter is U6Tet promoter, the transactivator element is a Tet Repressor element, and the exogenous factor is tetracycline or a derivative thereof. In some embodiments, the inducible RNA polymerase promoter is a TRE promoter and the transactivator element is a Tet-On element, and the exogenous factor is tetracycline or a derivative thereof.

In some embodiments, a flexible linker connects the inducible degron element to the immunosuppressive factor. In some embodiments, the flexible linker is selected from the group consisting of (GSG)ₙ, (GGGS)ₙ, and (GGGSGGGS)ₙ, wherein n is 1-10.

In some embodiments, the step (b) comprises introducing into the isolated cell a single nucleic acid construct comprising a promoter operably linked to the nucleic acid.

In some embodiments, the promoter is a constitutive promoter selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter. In some embodiments, the first, second and/or third promoters are constitutive promoters, each independently selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

In some embodiments, the construct comprises from 5′ end to 3′ end: a U6Tet promoter, a shRNA sequence targeting CD47, an EF1a promoter, and a Tet Repressor element, and wherein the exogenous factor is tetracycline or a derivative thereof.

In some embodiments, the construct further comprises a vector backbone for lentiviral expression.

In some embodiments, the inducible degron element is selected from the group consisting of a ligand inducible degron element, a peptidic degron element, and a peptidic proteolysis targeting chimera (PROTAC) element. In some embodiments, the ligand inducible degron element is selected from a small molecule-assisted shutoff (SMASH) degron element, Shield-1 responsive degron element, auxin responsive degron element, and a rapamycin responsive degron element. In some embodiments, the ligand inducible degron element is a small molecule-assisted shutoff (SMASH) degron element and the exogenous factor is asunaprevir.

In some embodiments, the construct further comprises a 5′ homology arm and a 3′ homology arm for targeted integration to a safe harbor locus selected from the group consisting of an AAVS1 locus, a CLBYL locus, a CXCR4 locus, a Rosa26 locus, and a CCR5 locus.

In some embodiments, the isolated cell is an isolated human cell further comprising deletion or reduced expression of MHC class I human leukocyte antigens and/or deletion or reduced expression of MHC class II human leukocyte antigens compared to an unmodified human cell. In some embodiments, the isolated human cell further comprises deletion or reduced expression of CIITA, B2M, and/or NLRC5.

In some embodiments, the isolated human cell is hypoimmunogenic and either a stem cell or a differentiated cell thereof, wherein the stem cell is selected from the group consisting of an embryonic stem cell, a pluripotent stem cell, an induced pluripotent stem cell, an adult stem cell, and wherein the differentiated cell is selected from the group consisting of a cardiac cell, liver cell, kidney cell, pancreatic cell, neural cell, immune cell, mesenchymal cell, and endothelial cell. In some embodiments, the isolated human cell is hypoimmunogenic. In some embodiments, the isolated human cell is a stem cell. In some instances, the stem cell is selected from the group consisting of an embryonic stem cell, a pluripotent stem cell, an induced pluripotent stem cell, and an adult stem cell.

In another aspect, provided herein is a construct comprising from 5′ end to 3′ end: an inducible RNA polymerase promoter; an shRNA sequence targeting an immunosuppressive factor; a constitutive promoter; and a transactivator element corresponding to the inducible RNA polymerase promoter.

In one aspect, provided herein is a construct comprising from 5′ end to 3′ end: an inducible RNA polymerase promoter; an immune signaling factor gene; a promoter; and a transactivator element corresponding to the inducible RNA polymerase promoter.

In some embodiments, the inducible RNA polymerase promoter is a U6Tet promoter. In many embodiments, the inducible RNA polymerase promoter is a TRE promoter.

In some embodiments, the immunosuppressive factor of the construct is selected from the group consisting of CD47, CD24, CD200, HLA-G, HLA-E, HLA-C, HLA-E heavy chain, PD-L1, IDO1, CTLA4-Ig, C1-Inhibitor, IL-10, IL-35, FASL, Serpinb9, CCl21, and Mfge8. In some embodiments, the immune signaling factor of the construct is selected from the group consisting of B2M, MIC-A, MIC-B, HLA-A, HLA-B, HLA-C, RFXANK, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some embodiments, the promoter of the construct is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the construct comprises from 5′ end to 3′ end: a U6Tet promoter, a shRNA sequence targeting CD47, an EF1a promoter, and a Tet Repressor element.

In some embodiments, the construct comprises from 5′ end to 3′ end: a TRE promoter, an immune signaling factor gene, an EF1a promoter, and a Tet-On element.

In some embodiments, the construct further comprises a vector backbone for lentiviral expression.

Also provided is a composition comprising an isolated cell comprising any of the constructs described herein.

Also provided is a composition comprising an isolated cell comprising any of the constructs described herein, wherein the isolated cell is engineered to exogenously express the immunosuppressive factor. In some embodiments, the isolated cell overexpresses the immunosuppressive factor in the absence of the exogenous factor that activates the transactivator element. In some embodiments, the isolated cell is exposed to an exogenous factor to activate the transactivator element. In some embodiments, the isolated cell is a stem cell selected from the group consisting of an embryonic stem cell, a pluripotent stem cell, and an adult stem cell.

In some embodiments, the composition comprises isolated differentiated cells prepared by culturing any of the stem cells outlined herein under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

In some aspects, provided herein is a method of treating a patient in need of cell therapy comprising: (a) administering any composition outlined herein to a patient; and (b) exposing the composition to an exogenous factor to activate the inducible RNA polymerase promoter, thereby controlling immunogenicity of the cells of the composition.

Provided is a pluripotent stem cell comprising (i) reduced or silenced expression of MHC class I molecules and/or MHC class II molecules, (ii) overexpression of CD47, and (iii) a factor selected from the group consisting of: an inducible shRNA targeting CD47, an inducible degron element controlling CD47, or a SMASH degron element controlling CD47.

Also provided herein is a pluripotent stem cell comprising (i) reduced or silenced expression of B2M and CIITA, (ii) overexpression of CD47, and (iii) a factor selected from the group consisting of: an inducible shRNA targeting CD47, an inducible degron element controlling CD47, or a SMASH degron element controlling CD47.

Additionally, outlined herein is a pluripotent stem cell comprising (i) reduced or silenced expression of MHC class I molecules and/or MHC class II molecules, (ii) overexpression of CD47, (iii) a Cas9 or a variant thereof, and (iv) an inducible guide RNA targeting CD47.

Also, outlined herein is a pluripotent stem cell comprising (i) reduced or silenced expression of B2M and CIITA, (ii) overexpression of CD47, (iii) a Cas9 or a variant thereof, and (iv) an inducible guide RNA targeting CD47.

Furthermore, outlined herein is a pluripotent stem cell comprising (i) reduced or silenced expression of MHC class I molecules and/or MHC class II molecules, (ii) overexpression of CD47, and (iii) an inducible protein degradation system for modulating expression of CD47 selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

Moreover, outlined herein is a pluripotent stem cell comprising (i) reduced or silenced expression of B2M and CIITA, (ii) overexpression of CD47, and (iii) an inducible protein degradation system for modulating expression of CD47 selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

Provided herein is a pluripotent stem cell comprising (i) reduced or silenced expression of MHC class I molecules and/or MHC class II molecules, (ii) overexpression of CD47, and (iii) an RNA regulation system for modulating expression of CD47 selected from the group consisting of an inducible shRNA, an inducible siRNA, a CRISPR interference (CRISPRi), and a RNA targeting nuclease system.

Provided herein is a pluripotent stem cell comprising (i) reduced or silenced expression of B2M and CIITA, (ii) overexpression of CD47, and (iii) an RNA regulation system for modulating expression of CD47 selected from the group consisting of an inducible shRNA, an inducible siRNA, a CRISPR interference (CRISPRi), and a RNA targeting nuclease system.

Provided herein is a pluripotent stem cell comprising (i) reduced or silenced expression of MHC class I molecules and/or MHC class II molecules, (ii) overexpression of CD47, and (iii) a DNA regulation system for modulating expression of CD47 selected from the group consisting of a tissue specific promoter expression system, an inducible promoter expression system, a molecule regulated riboswitch system, and an inducible nuclease-based genome editing system.

Provided herein is a pluripotent stem cell comprising (i) reduced or silenced expression of B2M and CIITA, (ii) overexpression of CD47, and (iii) a DNA regulation system for modulating expression of CD47 selected from the group consisting of a tissue specific promoter expression system, an inducible promoter expression system, a molecule regulated riboswitch system, and an inducible nuclease-based genome editing system.

Provided herein is a pluripotent stem cell comprising (i) reduced or silenced expression of MHC class I molecules and/or MHC class II molecules, (ii) overexpression of CD47, and (iii) an inducible system for modulating expression of CD47. In some embodiments, the inducible system decreases or reduces expression of CD47 in the cell.

Also, provided herein is a pluripotent stem cell comprising (i) reduced or silenced expression of B2M and CIITA. (ii) overexpression of CD47, and (iii) an inducible system for modulating expression of CD47. In some embodiments, the inducible system decreases or reduces expression of CD47.

In some instances, provided is a differentiated cell derived from any of the pluripotent stem cells outlined, wherein the differentiated cell is selected from the group consisting of a cardiac cell, liver cell, kidney cell, pancreatic cell, neural cell, immune cell, mesenchymal cell, and endothelial cell.

In one aspect, outlined is a construct comprising from 5′ end to 3′ end: a promoter, an inducible degron element, an optional sequence encoding a flexible linker, and an immunosuppressive factor gene.

In another aspect, outlined is a construct comprising from 5′ end to 3′ end: a promoter, an immunosuppressive factor gene, an optional sequence encoding a flexible linker, and an inducible degron element.

In some embodiments, the promoter is a constitutive promoter selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In many embodiments, the flexible linker is selected from the group consisting of consisting of (GSG)ₙ(SEQ ID NO:3), (GGGS)ₙ (SEQ ID NO:1), and (GGGSGGGS)ₙ (SEQ ID NO:2), wherein n is 1-10

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

In some embodiments, the inducible degron element is selected from the group consisting of a ligand inducible degron element, an inducible peptidic degron element, and a peptidic proteolysis targeting chimera (PROTAC) element. In some embodiments, the ligand inducible degron element is selected from a small molecule-assisted shutoff (SMASH) degron element, Shield-1 responsive degron element, auxin responsive degron element, and rapamycin responsive degron element.

In some embodiments, the construct further comprises a 5′ homology arm and a 3′ homology arm for targeted integration to a genomic safe harbor locus selected from the group consisting of an AAVS1 locus, a CLBYL locus, a CXCR4 locus, a Rosa26 locus, and a CCR5 locus.

Provided is a composition comprising an isolated cell comprising any of the constructs described.

In some embodiments, the isolated cell is selected from the group consisting of a stem cell, embryonic stem cell, pluripotent stem cell, and adult stem cell.

Provided is a composition comprising isolated differentiated cells prepared by culturing any of the stem cells described under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

In some aspect, provided herein is a method of treating a patient in need of cell therapy comprising: (a) administering the composition described to the patient; and (b) exposing the composition to an exogenous factor to activate the inducible degron element, thereby controlling immunogenicity of the cells of the composition.

In some aspect, provided herein is a composition comprising an isolated cell comprising a DNA targeted nuclease system for controlling immunogenicity of the cell comprising: (a) a first element comprising from 5′ end to 3′ end: a first promoter and an immunosuppressive factor gene; (b) a second element comprising from 5′ end to 3′ end: a second promoter and a nucleic acid sequence encoding Cas9 or a variant thereof; and (c) a third element comprising from 5′ end to 3′ end: an inducible RNA polymerase promoter, a guide RNA (gRNA) sequence targeting the immunosuppressive factor, a third promoter, and a transactivator element corresponding to the inducible promoter. In certain embodiments, immunogenicity of the cell is controllable upon exposing the cell to an exogenous factor to induce activity of the transactivator element. In some embodiments, the inducible RNA polymerase promoter is a U6Tet promoter, the transactivator element is a Tet Repressor element, and the exogenous factor is tetracycline or a derivative thereof.

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

In some embodiments, the first, second and/or third promoters are constitutive promoters, each independently selected from the group consisting of an EF1A promoter, an EFS promoter, a CMV promoter, a CAGGS promoter, a SV40 promoter, a COPIA promoter, an ACT5C promoter, a TRE promoter, a CBh promoter, a AGK promoter, and a UBC promoter.

In some embodiments, the isolated cell is an isolated human cell further comprising deletion or reduced expression of MHC class I human leukocyte antigens and/or deletion or reduced expression of MHC class II human leukocyte antigens compared to an unmodified human cell. In some embodiments, the isolated human cell further comprises deletion or reduced expression of CIITA, B2M, and/or NLRC5.

In some embodiments, the isolated human cell is hypoimmunogenic and a stem cell. In some instances, the stem cell is selected from the group consisting of an embryonic stem cell, a pluripotent stem cell, an induced pluripotent stem cell, and an adult stem cell.

Provided herein is a composition comprising isolated differentiated cells prepared by culturing the stem cell described under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

In one aspect, provided is a method of treating a patient in need of cell therapy comprising: (a) administering the composition described above; and (b) exposing the composition to an exogenous factor to activate the inducible RNA polymerase promoter, thereby controlling immunogenicity of the cells of the composition.

In one aspect, provided is a composition comprising an isolated mammalian cell comprising a modification comprising a recombinant nucleic acid sequence encoding a system for conditional expression of one or more immunosuppressive factors.

In one aspect, provided is a composition comprising an isolated mammalian cell comprising a recombinant nucleic acid sequence encoding a system for conditional expression of one or more immune signaling factors.

In some embodiments, the expression of the one or more immunosuppressive factors is controllable by an exogenous factor. In some embodiments, the expression of the one or more immune signaling factors is controllable by an exogenous factor.

In some embodiments, the system comprises an inducible protein degradation system to reduce protein levels of the one or more immunosuppressive factors. In some embodiments, the inducible protein degradation system is selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation. In some embodiments, the system comprises a RNA regulation system to controllably reduce RNA levels of the one or more immunosuppressive factors. In some embodiments, the RNA regulation system is selected from the group consisting of an inducible shRNA, an inducible siRNA, a CRISPR interference (CRISPRi), and a RNA targeting nuclease system. In some embodiments, the RNA regulation system is controllable by a ligand inducible transcription factor, a SynNotch receptor, or a ligand regulated riboswitch.

In some embodiments, the system comprises a DNA regulation system to reduce expression levels of the one or more immunosuppressive factors that is selected from the group consisting of a tissue-specific promoter expression system, an inducible promoter expression system, a molecule regulated riboswitch system, and an inducible nuclease-based genome editing system.

In some embodiments, the inducible promoter expression system comprises a U6Tet promoter and a Tet Repressor element.

In some embodiments, the system comprises an inducible protein stabilization system to increase protein levels of the one or more immune signaling factors.

In some embodiments, the inducible protein stabilization system comprises a ligand-inducible protein stabilization system and a small molecule-inducible protein stabilization system.

In some embodiments, the system comprises an RNA regulation system to increase RNA levels of the one or more immune signaling factors.

In some embodiments, the RNA regulation system comprises a CRISPR activation (CRISPRa) system.

In some embodiments, the system comprises a DNA regulation system to increase expression levels of the one or more immune signaling factors.

In some embodiments, the DNA regulation system comprises one selected from the group consisting of a CRISPR activation (CRISPRa) system, a tissue-specific promoter, an inducible promoter, and a molecule regulated riboswitch system.

In some embodiments, the tissue-specific promoter is selected from the group consisting of a cardiac cell-specific promoter, hepatocyte-specific promoter, kidney cell-specific promoter, pancreatic cell-specific promoter, neural cell-specific promoter, immune cell-specific promoter, mesenchymal cell-specific promoter, and endothelial cell-specific promoter.

In some embodiments, the inducible promoter comprises a TetOn system.

In some embodiments, the molecule regulated riboswitch system comprises a theophylline regulated riboswitch or a guanine regulated riboswitch.

In some embodiments, the inducible nuclease-based genome editing system comprises one selected from the group consisting of CRISPR genome editing comprising an inducible guide RNA targeting the one or more immunosuppressive factors, inducible TALEN genome editing, inducible ZFN genome editing, and small molecule enhanced CRISPR-based genome editing.

In some embodiments, the one or more immunosuppressive factors are selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

In some embodiments, the one or more immune signaling factors are selected from the group consisting of beta-2-microglobulin (B2M), MHC class I chain-related protein A (MIC-A), MHC class I chain-related protein B (MIC-B), HLA-A, HLA-B, HLA-C, RFXANK, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some embodiments, the isolated mammalian cell is an isolated human cell further comprising deletion or reduced expression of MHC class I human leukocyte antigens and/or deletion or reduced expression of MHC class II human leukocyte antigens compared to an unmodified human cell.

In some embodiments, the isolated human cell further comprises deletion or reduced expression of CIITA, B2M, and/or NLRC5.

In some embodiments, the isolated human cell is hypoimmunogenic and a stem cell. In some instances, the stem cell is selected from the group consisting of an embryonic stem cell, a pluripotent stem cell, an induced pluripotent stem cell, and an adult stem cell.

Provided is a composition comprising an isolated differentiated cell prepared by culturing any of the stem cells described under differentiation conditions appropriate for differentiation of a stem cell into a cell type selected from the group consisting of cardiac cells, liver cells, kidney cells, pancreatic cells, neural cells, immune cells, mesenchymal cells, and endothelial cells.

In another aspect, outlined is a method of treating a patient in need of cell therapy comprising: (a) administering the composition outlined; and (b) exposing the composition to an exogenous factor to control expression of the one or more immunosuppressive factors, thereby controlling immunogenicity of the cells of the composition.

In one aspect, outlined is a construct comprising from 5′ to 3′ end: (1) a safety switch transgene; (2) a ribosomal skipping sequence and/or a sequence encoding a linker; (3) a hypoimmunity gene. In another aspect, outlined is a construct comprising from 5′ to 3′ end: (1) a hypoimmunity gene; (2) a ribosomal skipping sequence or a linker; (3) a safety switch transgene.

In some embodiments, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

In some embodiments, the ribosomal skipping sequence comprises a sequence encoding an IRES sequence or a sequence encoding a 2A-coding sequence.

In some embodiments, the linker is selected from any one of the linkers provided in Table 3.

In some embodiments, the hypoimmunity gene is selected from the group consisting of: CD47, CD24, CD200, HLA-G, HLA-E, HLA-C, HLA-E heavy chain, PD-L1, ID01, CTLA4-Ig, IL-10, IL-35, FASL, Serpinb9, CCl21, and Mfge8.

In some embodiments, the construct further comprises a transcriptional regulatory element operably linked to the safety switch transgene and a polyadenylation sequence at the 3′ end of the hypoimmunity gene, or a transcriptional regulatory element operably linked to the hypoimmunity gene and a polyadenylation sequence at the 3′ end of the safety switch transgene.

In some embodiments, the transcriptional regulatory element of the construct is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

In some embodiments, the construct further comprises a vector backbone for lentiviral expression.

In some embodiments, provided is a method of delivering a construct into an isolated cell comprising transducing an isolated cell with a lentiviral construct comprising any construct outlined and selecting an engineered cell carrying the safety switch transgene and the hypoimmunity gene.

Provided herein is an isolated cell or a population thereof comprising a construct described. In some embodiments, the construct has been introduced into a target gene locus. In some embodiments, the gene locus is either a safe harbor locus selected from the group consisting of an AAVS1 locus, a CLBYL locus, a CXCR4 locus, a Rosa26 locus, and a CCR5 locus, or an immune signaling gene locus selected from the group consisting of B2M, HLA-A, HLA-B, HLA-C. HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D. In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and is a stem cell.

Also provided is a differentiated cell or a population thereof prepared by culturing the stem cell under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

Provided is a method of treating a patient in need of cell therapy comprising administering to patient the differentiated cell or the population thereof described. Provided is a method of treating a patient comprising activating a safety switch in a patient previously administered the differentiated cell or the population thereof described.

In one aspect, provided is a construct for homology directed repair into a safe harbor locus comprising from 5′ to 3′ end: (1) a first homology arm homologous to a first endogenous sequence of a safe harbor locus; (2) a safety switch transgene; (3) a ribosomal skipping sequence and/or a sequence encoding a linker; (4) an hypoimmunity gene; (5) a polyadenylation sequence; and (6) a second homology arm homologous to a second endogenous sequence of the safe harbor locus. In one aspect, provided is a construct for homology directed repair into a safe harbor locus comprising from 5′ to 3′ end: (1) a first homology arm homologous to a first endogenous sequence of an immune signaling gene locus; (2) a safety switch transgene; (3) a ribosomal skipping sequence and/or a sequence encoding a linker; (4) an hypoimmunity gene; (5) a polyadenylation sequence; and (6) a second homology arm homologous to a second endogenous sequence of the immune signaling gene locus. In one aspect, provided is a construct for homology directed repair into a safe harbor locus comprising from 5′ to 3′ end: (1) a first homology arm homologous to a first endogenous sequence of a safe harbor locus; (2) a safety switch transgene; (3) a ribosomal skipping sequence or a sequence encoding a linker; (4) an essential cell factor gene; (5) a polyadenylation sequence; and (6) a second homology arm homologous to a second endogenous sequence of the safe harbor locus. In another aspect, provided is a construct for homology directed repair into an immune signaling comprising from 5′ to 3′ end: (1) a first homology arm homologous to a first endogenous sequence of an immune signaling gene locus; (2) a safety switch transgene; (3) a ribosomal skipping sequence or a sequence encoding a linker; (4) an essential cell factor gene; (5) a polyadenylation sequence; and (6) a second homology arm homologous to a second endogenous sequence of the immune signaling gene locus. In yet another aspect, provided is a construct for homology directed repair into an essential cell factor gene locus comprising from 5′ to 3′ end: (1) a first homology arm homologous to a first endogenous sequence of an essential cell factor gene locus; (2) a sequence encoding a linker; (3) a safety switch transgene; and (4) a second homology arm homologous to a second endogenous sequence of the essential cell factor gene locus.

In some embodiments, the hypoimmunity gene is selected from the group consisting of CD47, CD24, CD200, HLA-G, HLA-E, HLA-C, HLA-E heavy chain, PD-L1, IDO1 CTLA4-Ig, IL-10, IL-35, FASL, Serpinb9, CCl21, and Mfge8.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

In some embodiments, the safe harbor locus is selected from the group consisting of an AAVS1 locus, a CLBYL locus, a CXCR4 locus, a Rosa26 locus, and a CCR5 locus.

In some embodiments, the immune signaling gene locus is selected from the group consisting of an B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAE11L/ULBP6, RAET1N/ULBP3, and other ligands of NKG2D.

In some embodiments, the immune signaling gene locus is selected from the group consisting of B2M, HLA-A, HLA-B, HLA-C, HLA-D, and HLA-E.

In some embodiments, the ribosomal skipping sequence comprises a sequence encoding an IRES sequence or a sequence encoding a 2A-coding sequence.

In some embodiments, the 2A-coding sequence is selected from the group consisting of T2A, P2A, E2A, and F2A.

In some embodiments, the construct enables a targeting nuclease to cleave the safe harbor locus or the immune signaling gene locus, thereby allowing the construct to recombine into the locus by homology directed repair.

In some embodiments, the construct enables a targeting nuclease to cleave the essential cell factor gene locus, thereby allowing the construct to recombine into the locus by homology directed repair.

In some embodiments, the construct further comprises a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter located at the 5′ end of the safety switch transgene.

In some embodiments, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

In some embodiments, the linker is selected from any one of the linkers provided in Table 3.

Outlined herein is an isolated cell or a population thereof comprising a safety switch transgene and a hypoimmunity gene integrated into a safe harbor locus or an immune signaling gene locus, wherein any of the constructs above has recombined into the endogenous safe harbor locus of a cell, or wherein any of the constructs above has recombined into the endogenous immune signaling gene locus of a cell. Outlined herein is an isolated cell or a population thereof comprising a safety switch transgene and an essential cell factor gene integrated into a safe harbor locus or an immune signaling gene locus, wherein any of the constructs above has recombined into the endogenous safe harbor locus of a cell, or wherein any of the constructs above has recombined into the endogenous immune signaling gene locus of a cell, and wherein the cell or the population thereof is unable to express the essential cell factor from the endogenous locus.

In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M and/or NLRC5. In some embodiments, the isolated cell is hypoimmunogenic and a stem cell. In some instances, the stem cell is selected from the group consisting of an embryonic stem cell, a pluripotent stem cell, an induced pluripotent stem cell, and an adult stem cell.

Provided is a differentiated cell or a population thereof prepared by culturing the any stem cell outlined herein under differentiation conditions appropriate for differentiation of the stem cell into a cell type selected from the group consisting of cardiac cells, liver cell, kidney cells, pancreatic cells, neural cells, immune cells, mesenchymal cells, and endothelial cells.

Disclosed is a method of treating a patient in need of cell therapy comprising administering to a patient the differentiated cell or the population thereof outlined. Also disclosed is a method of treating a patient comprising activating a safety switch in a patient previously administered the differentiated cell or the population thereof outlined.

Provided is a homology independent donor construct comprising from 5′ to 3′ end: (1) a 5′ long terminal repeats (LTR) comprising a left element (LE); (2) a splice acceptor-viral 2A peptide (SA-2A) element; (3) a safety switch transgene; (4) a ribosomal skipping sequence or sequence encoding a linker; (5) a hypoimmunity gene; (6) a polyadenylation sequence; and (7) 3′ LTR comprising a right element (RE).

Provided is a homology independent donor construct comprising from 5′ to 3′ end: (1) a 5′ long terminal repeats (LTR) comprising a left element (LE); (2) a splice acceptor-viral 2A peptide (SA-2A) element; (3) a safety switch transgene; (4) a ribosomal skipping sequence or a sequence encoding a linker; (5) an essential cell factor gene; (6) a polyadenylation sequence; and (7) 3′ LTR comprising a right element (RE).

Provided is a homology independent donor construct comprising from 5′ to 3′ end: (1) a 5′ long terminal repeats (LTR) comprising a left element (LE); (2) a splice acceptor-viral 2A peptide (SA-2A) element; (3) an essential cell factor gene; (4) a ribosomal skipping sequence or a sequence encoding a linker; (5) a safety switch transgene; (6) a polyadenylation sequence; and (7) 3′ LTR comprising a right element (RE).

In some embodiments, the hypoimmunity gene is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, and Mfge8.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

In some embodiments, the construct is configured to integrate into a target gene locus of an isolated cell to disrupt expression of the target gene.

In some embodiments, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

In some embodiments, the target gene locus is an immune signaling gene locus selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D. In some embodiments, the target gene locus is immune signaling gene locus selected from the group consisting of B2M, HLA-A, HLA-B, HLA-C, HLA-D, and HLA-E. In some embodiments, the target gene locus is a safe harbor locus selected from the group consisting of an AAVS1 locus, a CLBYL locus, a CXCR4 locus, a Rosa26 locus, and a CCR5 locus.

Outlined herein is an isolated cell or a population thereof comprising any of the constructs described, wherein the construct has integrated into an endogenous target gene to disrupt expression target gene expression in the isolated cell. Outlined herein is the isolated cell or the population thereof, wherein the isolated cell is unable to express the essential cell factor from the endogenous loci.

In some embodiments, the construct has integrated into the target gene at a nuclease or transposase target site. In some embodiments, one allele of the target gene are disrupted a nuclease or transposase targeting. In some embodiments, both alleles of the target gene are disrupted by a nuclease or transposase targeting. In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and a stem cell. In some instances, the stem cell is selected from the group consisting of an embryonic stem cell, a pluripotent stem cell, an induced pluripotent stem cell, and an adult stem cell.

In some aspect, provided herein is a differentiated cell or a population thereof prepared by culturing the stem cell outlined under differentiation conditions appropriate for differentiation of the stem cell into a cell type selected from the group consisting of cardiac cells, liver cells, kidney cells, pancreatic cells, neural cells, immune cells, mesenchymal cells, and endothelial cells. In some embodiments, provided is a method of treating a patient in need of cell therapy comprising administering to a patient the differentiated cell or the population thereof. In some embodiments, provided is a method of treating a patient comprising activating the safety switch in the patient previously administered the differentiated cell or the population thereof.

Provided herein is an isolated cell or a population thereof comprising an essential cell factor gene operably linked to a sequence encoding a linker that is operably linked to a safety switch transgene.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein. In some embodiments, the linker is selected from any one of the linkers provided in Table 3.

In some embodiments, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD30 transgene, and CD16 transgene.

In some aspect, provided herein is a recombinant peptide epitope fusion protein comprising: (1) a hypoimmunity factor selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof; and (2) a surface-exposed peptide epitope heterologous to the hypoimmunity factor selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

In some aspect, provided herein is a construct encoding a recombinant peptide epitope fusion protein comprising: (1) a sequence encoding a hypoimmunity factor selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof; and (2) a sequence encoding a surface-exposed peptide epitope heterologous to the hypoimmunity factor selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof, the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In some embodiments, the hypoimmunity factor and/or the peptide epitope is at the N-terminus of the fusion protein.

In some embodiments, the protein further comprises a linker connecting the hypoimmunity factor and the peptide epitope and/or located at the N-terminus or C-terminus of the fusion protein. In some embodiments, the linker is selected from any one of the linkers provided in Table 3.

In some embodiments, the sequence encoding the hypoimmunity factor of the construct is 5′ of the sequence encoding the peptide epitope and/or the sequence encoding the peptide epitope is at the 5′ of the sequence encoding the hypoimmunity factor.

In some embodiments, the construct further comprises a sequence encoding a linker connecting the sequence encoding the hypoimmunity factor and the sequence encoding the peptide epitope and/or located at the N-terminus or C-terminus of the fusion protein. In some embodiments, the linker is selected from any one of the linkers provided in Table 3.

In some embodiments, the construct further comprises a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter. In some embodiments, the construct further comprises a first homology arm and a second homology arm homologous to a target gene locus for CRISPR-based homology directed repair. In some embodiments, the construct further comprises a vector backbone for lentiviral expression.

Also provided is a method of delivering a construct into an isolated cell comprising transducing an isolated cell with a lentiviral construct comprising a construct of described herein; and selecting an engineered cell expressing a recombinant peptide epitope fusion protein. In some embodiments, provided is a method comprising transducing an isolated cell with any of the constructs described and selected the isolated cell that expresses the recombinant peptide epitope fusion protein encoded by the construct.

Also provided is an isolated cell or a population thereof comprising a construct described herein. In some embodiments, the isolated cell is an isolated human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and is a stem cell. In some embodiments, the stem cell is selected from the group consisting of an embryonic stem cell, a pluripotent stem cell, an induced pluripotent stem cell, and an adult stem cell.

Further, outlined herein is a differentiated cell or a population thereof prepared by culturing the stem cell described under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

In some cases, provided is a method of treating a patient in need of cell therapy comprising administering to patient the differentiated cell or the population thereof described. In some cases, provided is a method of treating a patient comprising administering to a patient previously administered the differentiated cell or the population thereof an antibody that binds the peptide epitope. In some embodiments, the antibody mediates ADCC or CDC.

In some aspects, outlined is a recombinant CD47-internal-peptide epitope fusion protein comprising from N- to C-terminal: (1) a human CD47 fragment comprising a IgV domain of CD47; (2) a first linker; (3) a heterologous peptide epitope; (4) a second linker; and (5) a human CD47 transmembrane domain.

In some embodiments, the human CD47 fragment comprising the IgV domain comprises amino acid residues 1-127 of the human CD47 protein. In some embodiments, the human CD47 transmembrane domain comprises amino acid residues 128-348 of the human CD47 protein. In some embodiments, the first and second linkers are selected from any one of the linkers provided in Table 3. In some embodiments, the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope. In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-IL2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In another aspect, disclosed is a construct comprising from 5′ to 3′ end: (1) a transcriptional regulatory element; (2) a sequence encoding a human CD47 fragment comprising a IgV domain of CD47; (3) a first linker; (4) a sequence encoding a peptide epitope; (5) a second linker; and (6) a sequence encoding a human CD47 fragment comprising a transmembrane domain and C-terminus.

In some embodiments, the human CD47 fragment comprising the IgV domain comprises amino acid residues 1-127 of the human CD47 protein. In some embodiments, the human CD47 fragment comprising the transmembrane domain and C-terminus comprises amino acid residues 128-348 of the human CD47 protein. In some embodiments, the first and second linkers are selected from any one of the linkers provided in Table 3. In some embodiments, the peptide epitope encoded by the sequence of (4) of the construct is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope. In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-IL2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In some embodiments, the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

In some embodiments, the construct further comprises a first homology arm and a second homology arm homologous to a target gene locus for CRISPR-based homology directed repair. In some embodiments, the construct further comprises a vector backbone for lentiviral expression.

Provided is a method of delivering a construct into an isolated cell comprising transducing an isolated cell with a lentiviral construct comprising a construct; and selecting an engineered cell expressing a CD47-internal-peptide epitope fusion protein. In some embodiments, the method comprises transducing an isolated cell with any of the constructs described and selected the isolated cell that expresses the CD47-internal-peptide epitope fusion protein encoded by the construct. Also provided is an isolated cell or a population thereof comprising the construct.

In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and a stem cell. In some instances, the stem cell is an embryonic stem cell, a pluripotent stem cell, an induced pluripotent stem cell, and an adult stem cell.

Further, outlined herein is a differentiated cell or a population thereof prepared by culturing the stem cell described under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

In some cases, provided is a method of treating a patient in need of cell therapy comprising administering to patient the differentiated cell or the population thereof described. In some cases, provided is a method of treating a patient previously administered the differentiated cell or the population thereof comprising administering to a patient an antibody that binds the peptide epitope. In some embodiments, the antibody mediates ADCC or CDC.

In one aspect, provided is a construct comprising (1) a transcriptional regulatory element, (2) an essential cell factor gene, (3) a post-transcriptional or post-translational regulatory element, and (4) a polyadenylation sequence.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

In some embodiments, the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter. In some embodiments, the post-transcriptional regulatory element is a RNA regulation system selected from the group consisting of an inducible shRNA, an inducible siRNA, a CRISPR interference (CRISPRi), and a RNA targeting nuclease system. In some embodiments, the post-translational regulatory element is an inducible protein degradation system is selected from the group consisting of a small molecule-assisted shutoff (SMASH) system, a shield-1-inducible degron, an auxin-inducible degron, an IMid-inducible degron, a peptidic degron, a proteolysis targeting chimera, and an antibody for targeted degradation.

Also provided is an isolated cell comprising a recombinant essential cell factor under the control of a post-transcriptional or post-translational regulatory element, wherein the endogenous essential cell factor gene is inactivated and expression of the recombinant essential cell factor is controllable by an exogenous factor. In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein. In some embodiments, the post-transcriptional regulatory element is a RNA regulation system selected from the group consisting of an inducible shRNA, an inducible siRNA, a CRISPR interference (CRISPRi), and a RNA targeting nuclease system. In some embodiments, the post-translational regulatory element is an inducible protein degradation system is selected from the group consisting of a small molecule-assisted shutoff (SMASH) system, a shield-1-inducible degron, an auxin-inducible degron, an IMid-inducible degron, a peptidic degron, a proteolysis targeting chimera, and an antibody for targeted degradation. In some embodiments, the isolated cell is an autologous human cell or an allogeneic human cell.

In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and selected from the group consisting of a stem cell and a differentiated cell.

In yet another aspect, provided herein is a bicistronic construct comprising from 5′ to 3′ end: (1) a transcriptional regulatory element; (2) a sequence encoding a surface-exposed peptide epitope; (3) a ribosomal skipping sequence; and (4) a sequence encoding a hypoimmunity factor. In some embodiments, the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof. In some embodiments, the surface-exposed peptide epitope encoded by the sequence of (2) of the construct is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope. In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof, the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In some embodiments, the ribosomal skipping sequence comprises a sequence encoding an IRES sequence or a sequence encoding a 2A-coding sequence.

In some embodiments, the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

In some embodiments, the construct further comprises a first homology arm and a second homology arm homologous to a target gene locus for CRISPR-based homology directed repair. In some embodiments, the construct further comprises a vector backbone for lentiviral expression.

In some embodiments, outlined is a method of delivering a construct into an isolated cell comprising transducing an isolated cell with a lentiviral construct comprising a construct described; and selecting an engineered cell expressing a hypoimmunity factor and a peptide epitope. In some embodiments, the method comprises transducing an isolated cell with the construct described; and selecting the isolated cell expressing the hypoimmunity factor and the peptide epitope both encoded by the construct. Also provided is an isolated cell or a population thereof comprising a construct of described.

In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5.

In some embodiments, the isolated cell is hypoimmunogenic and a stem cell. In some instances, the stem cell is selected from the group consisting of an embryonic stem cell, a pluripotent stem cell, an induced pluripotent stem cell, and an adult stem cell. In some embodiments, provided is a differentiated cell or a population thereof prepared by culturing the stem cell described under differentiation conditions appropriate for differentiation of the stem cell into a cell type selected from the group consisting of cardiac cells, liver cells, kidney cells, pancreatic cells, neural cells, immune cells, mesenchymal cells, and endothelial cells. In some instances, provided is a method of treating a patient in need of cell therapy comprising administering to patient the differentiated cell or the population thereof. In some instances, provided is a method of treating a patient comprising administering to a patient previously administered the differentiated cell or the population thereof an antibody that binds the peptide epitope. In some cases, the antibody mediates ADCC or CDC.

In one aspect, provided is a pluripotent stem cell comprising (i) reduced or silenced expression of MHC class I molecules and/or MHC class II molecules, (ii) a safety switch transgene and (iii) a hypoimmunity gene, wherein expression of the safety switch transgene modulates expression of the hypoimmunity gene.

In another aspect, provided is a pluripotent stem cell comprising (i) reduced or silenced expression of B2M and CIITA. (ii) overexpression of CD47, (iii) a safety switch transgene and (iv) a hypoimmunity gene, wherein expression of the safety switch transgene modulates expression of the hypoimmunity gene.

In yet another aspect, provided is a pluripotent stem cell comprising (i) reduced or silenced expression of MHC class I molecules and/or MHC class II molecules, (ii) a safety switch and (iii) a hypoimmunity factor, wherein expression of the safety switch modulates expression of the hypoimmunity factor.

In another aspect, provided is a pluripotent stem cell comprising (i) reduced or silenced expression of B2M and CIITA, (ii) overexpression of CD47, (iii) a safety switch and (iv) a hypoimmunity factor, wherein expression of the safety switch modulates expression of the hypoimmunity factor.

In one aspect, provided is a pluripotent stem cell comprising (i) reduced or silenced expression of MHC class I molecules and/or MHC class II molecules, and (ii) a hypoimmunity factor linked to a surface-exposed peptide epitope; wherein the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope, and the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

In one aspect, provided is a pluripotent stem cell comprising (i) reduced or silenced expression of B2M and CIITA, (ii) overexpression of CD47, and (iii) a hypoimmunity factor linked to a surface-exposed peptide epitope; wherein the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope, and the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

In one aspect, provided is a construct comprising from 5′ to 3′ end: (1) a safety switch transgene; (2) a ribosomal skipping sequence and/or a sequence encoding a linker; and (3) an essential cell factor gene. In some aspects, provided is a construct comprising from 5′ to 3′ end: (1) an essential cell factor gene; (2) a ribosomal skipping sequence or a linker; and (3) a safety switch transgene.

In some embodiments, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

In some embodiments, the ribosomal skipping sequence comprises a sequence encoding an IRES sequence or a sequence encoding a 2A-coding sequence.

In some embodiments, the linker is selected from any one of the linkers provided in Table 3.

In some embodiments, the hypoimmunity gene is selected from the group consisting of: CD47, CD24, CD200, HLA-G, HLA-E, HLA-C, HLA-E heavy chain, PD-L1, ID01, CTLA4-Ig, IL-10, IL-35, FASL, Serpinb9, CCl21, and Mfge8.

In some embodiments, the construct further comprises a transcriptional regulatory element operably linked to the safety switch transgene and a polyadenylation sequence at the 3′ end of the hypoimmunity gene, or a transcriptional regulatory element operably linked to the hypoimmunity gene and a polyadenylation sequence at the 3′ end of the safety switch transgene.

In some embodiments, the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

In some embodiments, the construct further comprises a vector backbone for lentiviral expression.

Outlined is a method of delivering a construct into an isolated cell comprising transducing an isolated cell with a lentiviral construct comprising a construct described; and selecting an engineered cell carrying the safety switch transgene and the hypoimmunity gene. In some embodiments, provided is a method comprising transducing an isolated cell with the construct (e.g., the lentiviral construct) described; and selecting the isolated cell carrying the safety switch transgene and the hypoimmunity gene of the construct. Also provided is an isolated cell or a population thereof comprising any one of the constructs described.

In some embodiments, the construct has been introduced into a target gene locus. In some embodiments, the target gene locus is selected from the group consisting of a safe harbor locus selected from the group consisting of an AAVS1 locus, a CLBYL locus, a CXCR4 locus, a Rosa26 locus, and a CCR5 locus and an immune signaling gene locus selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some embodiments, the isolated cell is an engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and a stem cell. In some embodiments, the stem cell is selected from an embryonic stem cell, a pluripotent stem cell, an induced pluripotent stem cell, and an adult stem cell.

In another aspect, disclosed is a differentiated cell or a population thereof prepared by culturing any stem cell under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

In some embodiments, provided is a method of treating a patient in need of cell therapy comprising administering to patient the differentiated cell or the population thereof disclosed. In some embodiments, provided is a method of treating a patient previously administered the differentiated cell or the population thereof disclosed comprising activating a safety switch in the patient.

In another aspect, provided is a recombinant peptide epitope fusion protein comprising: (1) an essential cell factor; and (2) a surface-exposed peptide epitope heterologous to the essential cell factor.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, a spliceosome subunit protein, and membrane-bound forms thereof.

In some embodiments, the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14,18K322A, Hu14.18-1L2, Hu3F8, dinituximab, c.60C3-RLIc, and biosimilars thereof, the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In some embodiments, the essential cell factor is at the N-terminus of the fusion protein. In some embodiments, the peptide epitope is at the N-terminus of the fusion protein. In some embodiments, the protein further comprises a linker connecting the essential cell factor and the peptide epitope. In some embodiments, the protein further comprises a linker located at the N-terminus of the peptide epitope. In some embodiments, the linker is selected from any one of the linkers provided in Table 3.

In another aspect, provided is a construct encoding a recombinant peptide epitope fusion protein comprising: (1) a sequence encoding an essential cell factor; and (2) a sequence encoding a surface-exposed peptide epitope heterologous to the essential cell factor.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, a spliceosome subunit protein, and membrane-bound forms thereof. In some embodiments, the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope. In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of M0R208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-IL2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In some embodiments, the sequence encoding the essential cell factor is 5′ of the sequence encoding the peptide epitope.

In some embodiments, the sequence encoding the peptide epitope is at the 5′ of the sequence encoding the essential cell factor.

In some embodiments, the construct further comprises a sequence encoding a linker connecting the sequence encoding the essential cell factor and the sequence encoding the peptide epitope. In some embodiments, the construct further comprises a sequence encoding a linker located at the N-terminus or C-terminus of the fusion protein. In some embodiments, the linker is selected from any one of the linkers provided in Table 3. In some embodiments, the construct comprises a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

In some embodiments, the construct further comprises a first homology arm and a second homology arm homologous to a target gene locus for CRISPR-based homology directed repair. In some embodiments, the construct further comprises a vector backbone for lentiviral expression.

In some embodiments, outlined is a method of delivering a construct into an isolated cell comprising transducing an isolated cell with a lentiviral construct comprising a construct described; and selecting an engineered cell expressing a recombinant peptide epitope fusion protein. In other embodiments, the method comprises transducing an isolated cell with the lentiviral construct described; and selecting the isolated cell expressing the recombinant peptide epitope fusion protein in the construct. In some embodiments, an isolated cell or a population thereof comprises a construct mentioned herein.

In some embodiments, the isolated cell is an isolated engineered human cell further comprising deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and a stem cell. In some instances, the stem cell is selected from the group consisting of an embryonic stem cell, a pluripotent stem cell, an induced pluripotent stem cell, and an adult stem cell.

In some embodiments, provided is a differentiated cell or a population thereof prepared by culturing the stem cell described under differentiation conditions appropriate for differentiation of the stem cell into a cell type selected from the group consisting of cardiac cells, liver cells, kidney cells, pancreatic cells, neural cells, immune cells, mesenchymal cells, and endothelial cells. In some instances, provided is a method of treating a patient in need of cell therapy comprising administering to patient the differentiated cell or the population thereof. In some instances, provided is a method of treating a patient comprising administering to a patient previously administered the differentiated cell or the population thereof an antibody that binds the peptide epitope. In some cases, the antibody mediates ADCC or CDC.

Provided is a construct for homology directed repair into a safe harbor locus comprising from 5′ to 3′ end: (1) a first homology arm homologous to a first endogenous sequence of a safe harbor locus; (2) a transcriptional regulatory element; (3) an HSVtk safety switch transgene; (4) a ribosomal skipping sequence and/or a sequence encoding a linker; (5) a CD47 hypoimmunity gene; (6) a polyadenylation sequence; and (7) a second homology arm homologous to a second endogenous sequence of the safe harbor locus. Provided is a construct for homology directed repair into a safe harbor locus comprising from 5′ to 3′ end: (1) a first homology arm homologous to a first endogenous sequence of an immune signaling gene locus; (2) a transcriptional regulatory element; (3) an HSVtk safety switch transgene; (4) a ribosomal skipping sequence and/or a sequence encoding a linker; (5) an CD47 hypoimmunity gene; (6) a polyadenylation sequence; and (7) a second homology arm homologous to a second endogenous sequence of the immune signaling gene locus.

In some embodiments, the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter. In some embodiments, the construct further comprises a vector backbone for lentiviral expression.

Provided is an isolated cell or a population thereof comprising a safety switch transgene and a hypoimmunity gene integrated into a safe harbor locus or an immune signaling gene locus, wherein the construct of described herein has recombined into the endogenous safe harbor locus of the isolated cell or into the endogenous targeted gene locus of the isolated cell.

In some embodiments, the isolated cell further comprises deletion or reduced expression of MHC class I human leukocyte antigens and/or deletion or reduced expression of MHC class II human leukocyte antigens compared to an unmodified human cell. In some embodiments, the isolated cell further comprises deletion or reduced expression of CIITA, B2M, and/or NLRC5. In some embodiments, the isolated cell is hypoimmunogenic and a stem cell. In some instances, the stem cell is selected from the group consisting of an embryonic stem cell, a pluripotent stem cell, an induced pluripotent stem cell, and an adult stem cell.

Provided herein is a differentiated cell or a population thereof prepared by culturing the stem cell described under differentiation conditions appropriate for differentiation of any of the stem cells into pancreatic cells. In some embodiments, the pancreatic cells are beta-islet cells.

Outlined is a method of treating a patient in need of cell therapy comprising administering to a patient the differentiated cell or the population thereof disclosed, and activating the safety switch in a patient previously administered the differentiated cell or the population thereof as described.

Detailed descriptions of hypoimmunogenic cells, methods of producing thereof, and methods of using thereof are found in WO2016183041 filed May 9, 2015 and WO2018132783 filed Jan. 14, 2018, the disclosures including the sequence listings and Figures are incorporated herein by reference in their entirety.

Other objects, advantages and embodiments of the present technology will be apparent from the detailed description following.

### BRIEF DESCRIPTION OF THE DRAWINGS

FIG. 1 depicts data in a HEK293 cell line engineered to express mouse CD47, showing various knockdown efficiencies of different shRNA constructs. Downregulation of exogenous CD47 was by way of an inducible shRNA controlled by a TetO system.

FIG. 2 provides a schematic diagram of the small-molecule assisted shutoff (SMASH) system (see Hannah et al., Nature Chemical Biology 11637-638 (2015)) for inducing CD47 degradation. SMASH is a system using the hepatitis C virus (HCV) nonstructural protein 3 (NS3) protease and elements in the NS4A protein to effectively shut off expression of CD47 protein fused to a SMASH-tag with clinically tested HCV protease inhibitors. In the absence of protease inhibitor (asunaprevir), a cryptic degron sequence is excised, leading to an unmodified gene product. By addition of asunaprevir, the NS3 protease is inhibited, leading to the degradation of newly synthesized CD47 proteins fused to the degron sequence.

FIG. 3A-FIG. 3B provide schematic diagrams of a donor template for homology directed repair (HDR) into the AAVS1 genomic safe harbor locus. FIG. 2A shows a cassette containing an EF1a core promoter (EFS) and a SMASH tag fused to the human CD47 gene and inserted in between two 1000 bp homology arms to the AAVS1 genomic safe harbor locus, hereafter referred to as the AAVS1-EFS-SMASH-CD47-AAVS1 donor template. FIG. 2B shows that overexpression of CD47 is achieved by knocking in the AAVS1-EFS-SMASH-CD47-AAVS1 cassette into the AAVS1 genomic safe harbor locus.

FIG. 4 provides a summary of a study to assess the ability of the SMASH system to promote CD47 degradation. (Top) a schematic diagram of an expression construct including a EFS-SMASH-CD47 expression cassette. This expression construct includes an EF1a core promoter (EFS) operably linked to a human CD47 gene fused to a nucleic acid encoding a SMASH tag. (Bottom) iPSCs transduced with either the EFS-SMASH-CD47 expression cassette or control EFS-CD47 expression cassette were assessed for CD47 expression in the presence of different concentrations of asunaprevir for 48 hours.

FIG. 5 provides a schematic diagram of the Ligand-Induced Degradation (LID) system for inducing CD47 degradation (see Bonger et al., Nat Chem Biol. 7(8):531-537 (2012)). In the LID system, a protein of interest (POI e.g., CD47) is fused to a LID domain. The LID domain includes the FK506- and rapamycin-binding protein (FKBP) and a peptide degron fused to the C-terminus of the FKBP. FKBP is an enzyme possessing cis/trans prolyl isomerase activity and can active on a broad spectrum of substrate polypeptides. The peptide degron is capable of binding to the FKBP active site and is not detected by cellular degradation proteins when sequestered in the active site, thus rendering it a cryptic degron. In the absence of the small molecule, Shield-1, the POI-LID fusion protein is stable. In the presence of Shield-1, however, Shield-1 binds tightly to FKBP, thereby displacing the peptide degron and inducing rapid degradation of the LID and any fused partner protein (e.g., CD47).

FIG. 6 provides a summary of a study to assess the ability of the LID system to promote CD47 degradation. (Top) a schematic diagram of the expression construct including the EFS-CD47-LID expression cassette used in the study. This expression construct includes an EF1a core promoter (EFS) operably linked to a human CD47 gene fused to a nucleic acid encoding a LID domain. (Bottom) summary of an experiment wherein iPSCs were transduced with either the EFS-CD47-LID expression cassette or control EFS-CD47 expression cassette were assessed for CD47 expression in the presence of different concentrations of Shield-1 for 24 hours.

FIG. 7 provides a schematic of a hypoimmune cell with an activated safety switch such that the resulting cell that no longer expresses the hypoimmunity factor is recognized by immune cells and is cleared by the immune system.

FIG. 8A-FIG. 8D schematically shows that hypoimmune cells can be engineered to have hypoimmunity when modified by way of cell targeting (FIG. 8A), protein regulation (FIG. 8B), RNA regulation (FIG. 8C), and DNA regulation (FIG. 8D). Protein regulation, RNA regulation, and DNA regulation can be inducible thereby generating an inducible hypoimmune cell.

FIG. 9 schematically shows that expression of immunosuppressive factors is controlled by regulated degradation or knockdown. See, e.g., Liang, Qin, et al. “Linking a cell-division gene and a suicide gene to define and improve cell therapy safety.” Nature 563.7733 (2018): 701.

FIG. 10 shows that cells transduced with a lentivirus vector harboring an inducible shRNA targeting exogenous CD47 and at an MOI above 0.3 exhibited efficient knockdown of CD47.

FIG. 11 depicts architecture of co-dependent safety switch-hypoimmune molecule constructs. Coexpression of a safety switch and hypoimmune molecule is obtained through the expression of a polycistronic transcript whereby the hypoimmune molecule and safety switch separated by a ribosomal skipping sequence, such as an IRES, or a 2A self-cleaving peptide. The expression of the cassette is regulated by a promoter for genomic location independent transcriptional regulation or a splice acceptor to enable regulation of the payload by an endogenous promoter following targeted integration.

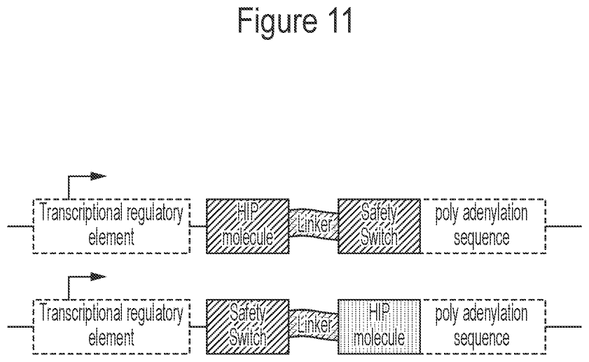

FIG. 12 schematically illustrates targeted genomic integration of a CD47-HSVtk fusion construct into the B2M locus for simultaneous disruption of B2M and expression of the safety switch cassette. HSVtk and CD47 are linked genetically within a polycistronic cassette via a P2A self-cleaving peptide. The cassette will be flanked by homology arms complementary to the B2M locus, allowing the cassette to be integrated via Cas9-induced HDR.

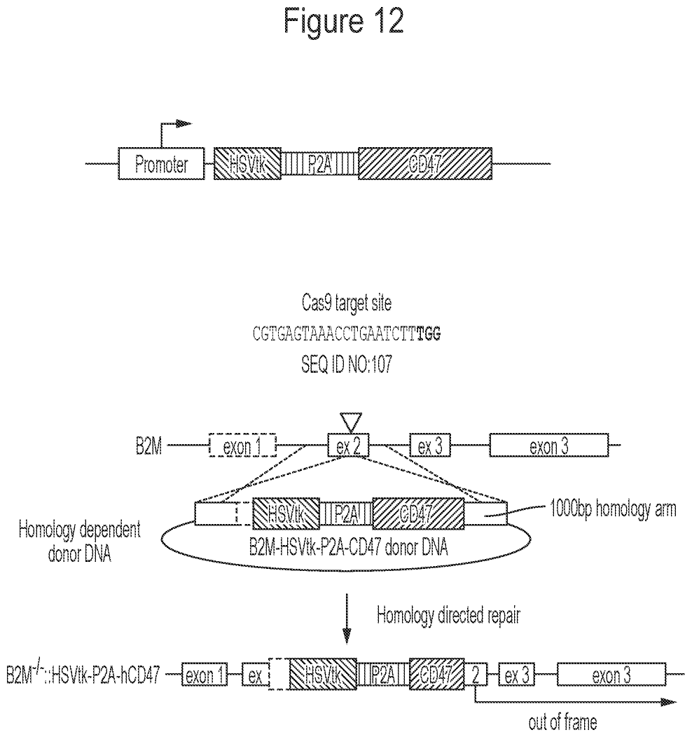

FIG. 13 schematically shows replacement of an essential gene with a synthetic essential gene-safety switch fusion. Linking safety switch expression to that of an essential gene ensures expression of the switch within viable cells. To directly link the expression of an essential gene to a safety switch without depending on the gene products of the essential endogenous locus, the essential gene cDNA (“SynEssentialGene”) is located within a cassette and fused to the safety switch. To require presence of the cassette and to place it under proper transcriptional regulation, the cassette harbors a splice acceptor 2A sequence to utilize the endogenous promoter and contains homology arms to the essential gene locus facilitating integration into the locus. By providing Cas9-sgRNA targeted to the essential gene locus, Cas9-induced HDR enables simultaneous disruption of the locus gene product and integration of the cassette, effectively replacing the native gene.

FIG. 14 schematically shows architecture of a CD47-ADCC/CDC amino terminal dependent safety switch. ADCC and CDC function via immune system effector cells recognizing an antibody bound to the extracellular surface of a cell. Expression of the epitope for which an antibody binds is sufficient to activate ADCC/CDC and can serve as a safety switch. The genetic fusion of an epitope to a hypoimmune molecule, such as CD47, encodes a failsafe for eliminating engineered hypoimmune cells. Peptidic epitopes, such as the fragment of CD20 recognized by rituximab, can be linked to extracellular hypoimmune molecules like CD47. Specifically, an N-terminal fusion of the CD20 epitope to CD47 sterically avails the epitope for rituximab binding without disrupting CD47 function. This same design can be applied to other hypoimmune molecules.

FIG. 15 schematically shows architecture of a CD47 variant harboring an internal CD20 epitope. The CD20 epitope can be inserted directly into CD47 downstream of the IgV domain to place the epitope between the IgV domain and nascent to the transmembrane domain. Placement downstream of the CD20 epitope downstream of the IgV domain renders the quarternary structure of the IgV intact.

FIG. 16 provides a summary of a study to assess the ability of cytosine demanise switch to induce cell death in an immune system dependent manner. (Top) a schematic diagram of the EFS-cytosine deaminase (CD)-CD47 bicistronic cassette used in the study. In this cassette, a nucleic acid encoding CD is located upstream of a nucleic acid encoding CD47. A 2A sequence is inserted between the CD and CD47 nucleic acids to ensure the two proteins are separate following translation. An EFS promoter is further included in the cassette for expression of the two proteins. (Middle chart) A chart showing the viability of EFS-CD-CD47 transduced cells and EFS-CD transduced cells (“CD”; suicide gene only, a control for the EFS-CD-CD47 transduced cells) in the presence of varying concentrations of 5-fluorocytosine (5-FC). (Bottom chart) compares CD47 expression in EFS-CD-CD47 transduced cells and CD cells by flow cytometry.

FIG. 17 is a schematic diagram depicting components and architecture of DNA cassettes encoding safety switches or essential cell factor molecules respectively or as a combined payload. In some embodiments, a payload refers to a safety switch linked to an essential cell factor. Heterologous genes harboring cDNAs for safety switches and essential cell factors contain several components: a transcriptional regulatory sequence such as a ubiquitous promoter or splice acceptor, an open reading frame (ORF) encoding the safety switch or essential cell factor, a polyadenylation sequence, and post-transcriptional or post-translational regulatory elements at the amino or carboxy terminus of the essential gene or the payload. Notably, examples of regulatory elements can be riboswitches for control of translation or chemically-destabilizable degron motifs that exist as a fusion protein with the payload.

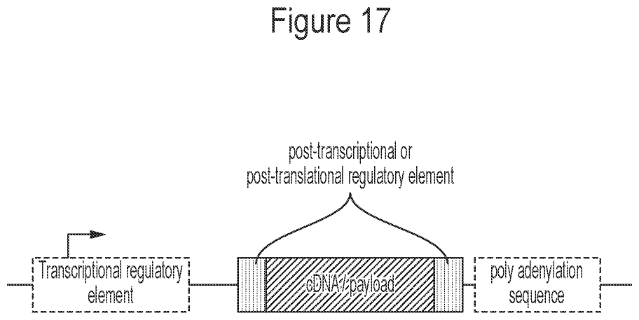

FIG. 18 is a schematic diagram illustrating architecture of co-dependent safety switch-essential cell factor constructs. Coexpression of a safety switch and essential cell factor molecule is obtained through the expression of a polycistronic transcript whereby the essential cell factor and safety switch separated by a ribosomal skipping sequence, such as an IRES, or a 2A self-cleaving peptide. The expression of the cassette is regulated by a promoter for genomic location independent transcriptional regulation or a splice acceptor to enable regulation of the payload by an endogenous promoter following targeted integration.

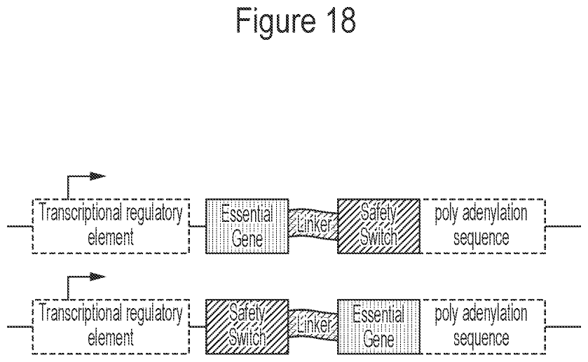

FIG. 19 is a schematic diagram illustrating integration of a safety switch into an endogenous essential locus to safeguard switch expression—organization of the locus post-targeting. The safety switch is integrated at the C terminus of an essential gene, such as a ribosomal or proteasomal gene, to ensure expression of the safety switch. The upstream of the switch is a linker sequence that encodes an iRES or 2A to allow proper separation of the safety switch and essential gene protein products. The stop codon is moved to be downstream of the safety switch.

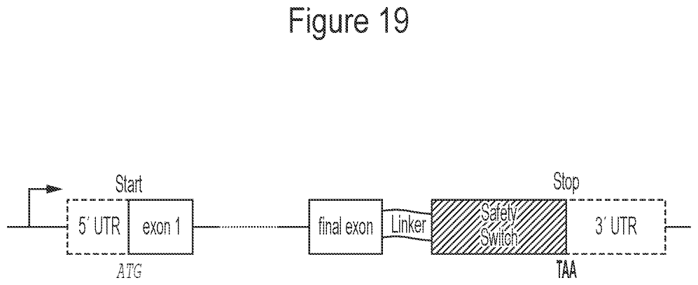

FIG. 20 is a schematic diagram illustrating targeted integration of a payload (e.g., safety switch and an essential gene) for co-expression of the safety switch and the essential gene into safe harbor loci. Donor DNAs encoding safety switch payloads as described above is integrated into a safe harbor locus, such as AAVS1, via targeted nuclease activity, such as the S. pyogenes Cas9-sgRNA complex. Integration occurs via homology directed repair (HDR). In some embodiments, the essential gene is knocked-out at its endogenous locus.

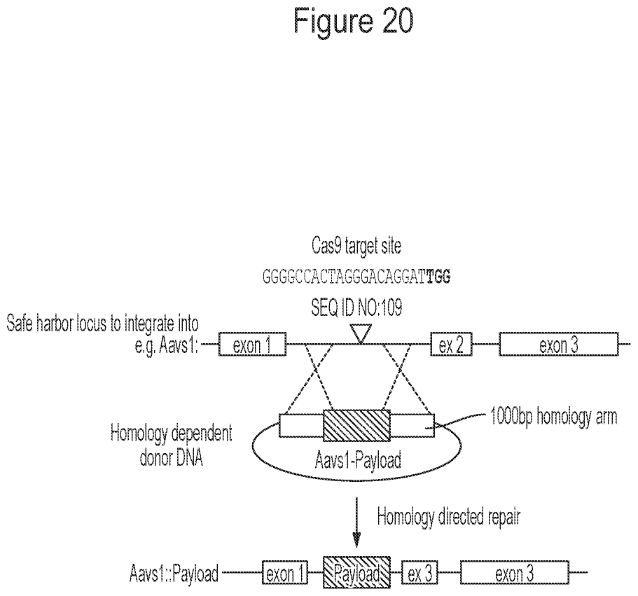

FIG. 21 is a schematic diagram illustrating simultaneous disruption of an immune-relevant locus and insertion of an essential cell factor or safety switch. Disruption of the immune-relevant locus, such as B2M, occurs via Cas9-induced HDR that incorporates the cassette encoding the safety switch or essential cell factor cassette. Incorporation of the cassette renders the endogenous gene product out of frame and results in expression of the cassette. Expression of the cassette is conferred by a promoter within the cassette or via the endogenous promoter through utilization of a splice-acceptor 2A sequence.

FIG. 22 is a schematic diagram illustrating integration of a safety switch or essential cell factor cassette into the carboxy terminus of an essential gene. As described in FIG. 3, cassettes encoding safety switches linked to essential cell factors are constructed to contain two homology arms that target the exogenous DNA for insertion at the carboxy terminus of the essential gene. Integration occurs via HDR mediated by Cas9-induced DNA repair.

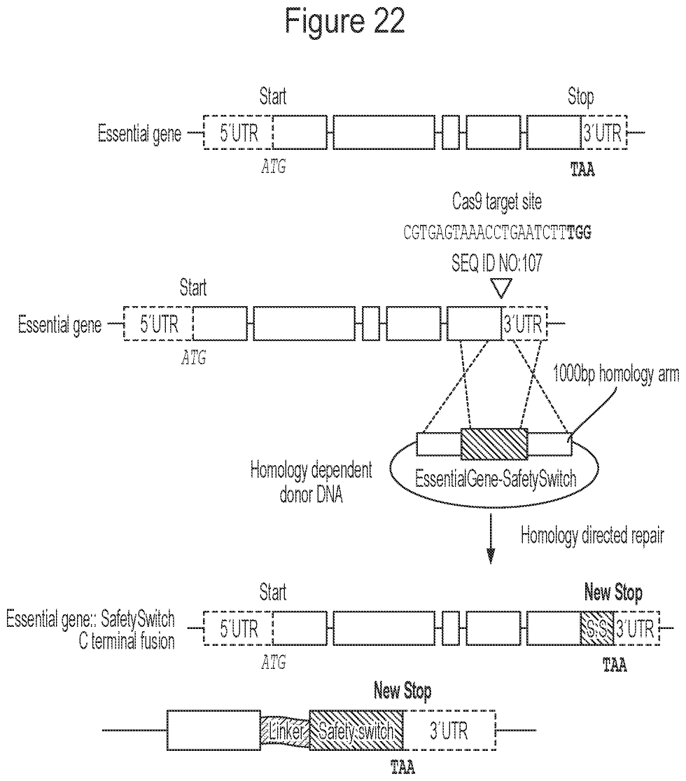

FIG. 23 is a schematic diagram illustrating targeted, homology independent integration of safety switches or essential cell factors. Donor DNAs encoding safety switch and essential cell factor payloads lacking homology arms are packaged as lentiviral genomes or transposons, facilitating integration via homology-independent DNA repair processes. Ligation of the exogenous DNA occurs at RNA-guided nuclease or transposase target sequences, and the expression of the cassette are regulated by the endogenous promoter using splice-acceptor 2A sequences.

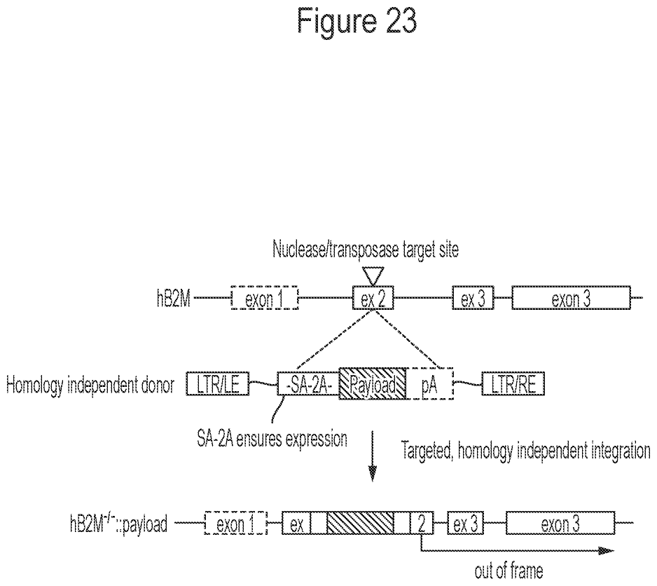

FIG. 24 is a schematic diagram illustrating targeted genetic disruption of an essential gene locus via nuclease activity. RNA-guided nucleases such as Cas9 are targeted to the PSMA3 locus, or another loci of interest, to facilitate the introduction of a frame-shifting mutation that disrupts proper transcription or translation of the protein product. In some embodiments (such as delivery of the safety-switch and co-expression construct to a safe harbor locus, immune signaling locus, or using lentiviral integration, the function of this switch requires inactivation of all copies of the essential gene at the endogenous locus, such that survival of the engineered cell is dependent on expression of the essential gene from the safe-harbor locus. The bottom panel depicts a strategy for inactivation of the endogenous locus using a CRISPR to introduce double strand breaks. The repair of these breaks by NHEJ or MMEJ leads to insertions or deletions that inactivate the essential gene at the endogenous locus.

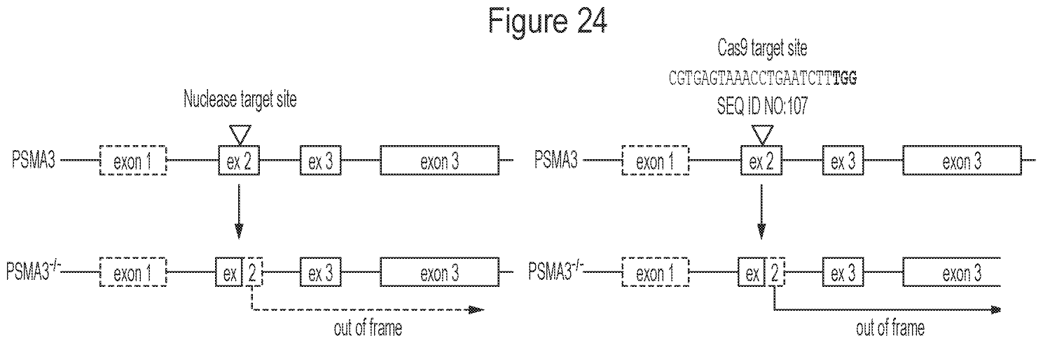

FIG. 25 is a schematic diagram illustrating integration of a post-transcriptional or post-translational regulatory element at the amino or carboxy terminus of an essential gene. This construct acts as a safety switch by providing exogenous control over expression of the essential gene.

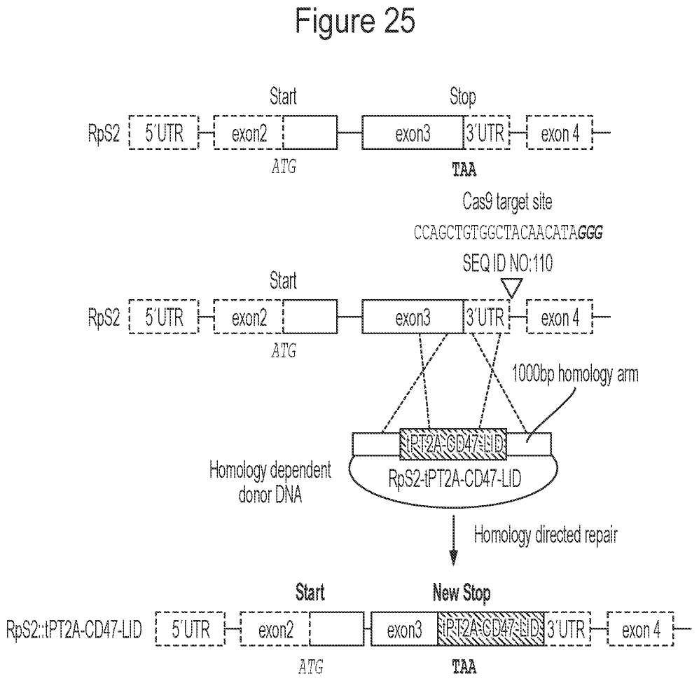

### DETAILED DESCRIPTION

### I. Introduction

Hypoimmune pluripotent stem cells (also referred to herein as “HIP cells”) and differentiated cells thereof that have been engineered to express immune regulator proteins and evade rejection by the host immune system hold significant promise for allogenic cell therapy. The introduction of safety switches to modulate the activity of such cells upon administration to a recipient subject is an important technology to improve the safety of these cell therapies. Described herein are embodiments of a safety switch based on regulating expression of an immunosuppressive factor (e.g., an hypoimmunity factor) in engineered cells. Provided herein are methods of regulating expression of immunosuppressive factors at either protein, RNA or DNA levels, and thereby functioning as a safety switch (e.g., conditional or inducible expression systems) for the cells. Also provided are pluripotent stem cells and derivative thereof comprising a modification for conditional expression of an immunosuppressive factor that is responsive to an exogenous signal such as a small molecule or biologic agent.

A key feature of HIP cells is their expression of immunosuppressive factors that function to suppress the host cell immune response to the engrafted cell population. In one embodiment, this safety switch is based on controllable expression of CD47. CD47 is a component of the innate immune system that functions as a “don't eat me” signal as part of the innate immune system to block phagocytosis by macrophages. In other embodiments, this safety switch

Provided herein are conditional or inducible hypoimmunogenic cells (e.g., conditional hypoimmunogenic pluripotent cells) that represents a viable source for any transplantable cell type. Such cells are protected from adaptive and innate immune rejection upon administration to a recipient subject by way of conditional expression of one or more immunogenicity factors. The expression of such immunogenicity factors is controlled by the activity of a conditional expression system. Non-limiting examples of a conditional expression system include an inducible protein degradation system, an inducible RNA regulatory system, and an inducible DNA regulation system.

In some embodiments, hypoimmunogenic cells outlined herein are not subject to an innate immune cell rejection prior to induction of the conditional expression system. In some instances, hypoimmunogenic cells are not susceptible to NK cell-mediated lysis prior to induction of the conditional expression system. In some instances, hypoimmunogenic cells are not susceptible to macrophage engulfment prior to induction of the conditional expression system. In some embodiments, hypoimmunogenic cells outlined herein are subject to an innate immune cell rejection upon induction of the conditional expression system. In some instances, hypoimmunogenic cells are susceptible to NK cell-mediated lysis upon induction of the conditional expression system. In some instances, hypoimmunogenic cells not susceptible to macrophage engulfment upon induction of the conditional expression system. In some embodiments, hypoimmunogenic cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation.

In some embodiments, provided herein are stem cells and/or differentiated derivatives thereof that conditionally evade immune rejection in an MHC-mismatched allogenic recipient. In some instances, differentiated cells produced from the stem cells outlined herein conditionally evade immune rejection when administered (e.g., transplanted or grafted) to MHC-mismatched allogenic recipient. In other words, the stem cells and/or differentiated cells derived from such stem cells are hypoimmunogenic in the absence of an exogenous factor that controls the activity of the conditional expression system targeting an exogenous immunogenicity factor expressed by the cells. In the presence of the exogenous factor, the exogenous immunogenicity factor is downregulated or degraded according to the conditional expression system. As such, the stem cells and/or differentiated cells derived from such stem cells are no longer hypoimmunogenic. In some cases, the cells do not have reduced immunogenicity (such as, at least 2.5%-99% less immunogenicity) compared to wild-type or non-engineered cell. In some cases, the cells have immunogenicity. In other words, such cells become immunogenic to a recipient subject and are thus cleared and/or targeted for cell death by the recipient subject's immune system.

In some embodiments, the stem cells described herein retain pluripotent stem cell potential and differentiation capacity.

### II. Definitions

The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In one embodiment, the safety switch is a ‘kill switch’ that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis.

As used herein to characterize a cell, the term “hypoimmunogenic” generally means that such cell is less prone to immune rejection by a subject into which such cells are transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some aspects, genome editing technologies are used to modulate the expression of MHC I and MHC II genes, and thus, generate a hypoimmunogenic cell. In some embodiments, a hypoimmunogenic cell evades immune rejection in an MHC-mismatched allogenic recipient. In some instance, differentiated cells produced from the hypoimmunogenic stem cells outlined herein evade immune rejection when administered (e.g., transplanted or grafted) to an MHC-mismatched allogenic recipient. In some embodiments, a hypoimmunogenic cell is protected from T cell-mediated adaptive immune rejection and/or innate immune cell rejection.

Hypoimmunogenicity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

The term “essential cell factor” is used herein refers to a protein or molecule that is necessary for cell survival and/or cell proliferation. Additional descriptions of essential cell factors or essential genes can be found, e.g., in Kabir et al., PloS One, 2017, 12, 5 e0178273; Hart et al., G3, 2017, 7, 2719-2727, Mair et al., Cell Reports, 2019, 27, 599-615; Wang et al., Science, 2015, 350(6264), 1096-1101; Yilmaz et al., Nat Cell Biol, 2018, 20, 610-619; Liu et al., Aging, 2019, 11(12):4011-4031; Ihry et al., Cell Reports, 2019, 27, 616-630; Bertomeu et al., Mol Cell Biol, 2018, 38(1):e00302-17; and Hart et al., Cell, 2015, 163, 1515-1526.

“Immunosuppressive factor” or “immune regulatory factor” as used herein include hypoimmunity factors and in some cases, also complement inhibitors. As used herein, the terms “immunosuppressive factor” and “hypoimmunity factor” are used interchangeably.

“Immune signaling factor” as used herein refers to, in some cases, a molecule, protein, peptide and the like that activates immune signaling pathways.

“Inducible expression system” as used herein refers a gene expression that can be controlled or induced by a ligand, small molecule, peptide, factor, agent, and the like. In some cases, the conditional gene expression system can turn on or turn off transcription in the presence of a ligand, small molecule, peptide, factor, agent, and the like. In some cases, the conditional gene expression system can activate a protein degradation pathway in response to the presence of a ligand, small molecule, peptide, factor, agent, and the like

“Degron element” as used herein refers to a subunit of a protein that regulates the degradation of the protein. In some instances, a degron comprises a sequence of amino acids, which provides a degradation signal that directs a polypeptide for cellular degradation. The degron may promote degradation of an attached polypeptide through either the proteasome or autophagy-lysosome pathways. In the fusion protein, the degron must be operably linked to the polypeptide of interest, but need not be contiguous with it as long as the degron still functions to direct degradation of the polypeptide of interest. Preferably, the degron induces rapid degradation of the polypeptide of interest. For a discussion of degrons and their function in protein degradation, see, e.g., Kanemaki et al. (2013) Pflugers Arch. 465(3):419-425, Erales et al. (2014) Biochim Biophys Acta 1843(1):216-221, Schrader et al. (2009) Nat. Chem. Biol. 5(11):815-822, Ravid et al. (2008) Nat. Rev. Mol. Cell. Biol. 9(9):679-690, Tasaki et al. (2007) Trends Biochem Sci. 32(11):520-528, Meinnel et al. (2006) Biol. Chem. 387(7):839-851, Kim et al. (2013) Autophagy 9(7):1100-1103, Varshaysky (2012) Methods Mol. Biol. 832:1-11, and Fayadat et al. (2003) Mol Biol Cell. 14(3):1268-1278; the contents herein incorporated by reference in their entirety.

“Safe harbor locus” as used herein refers to a gene locus that allows safe expression of a transgene or an exogenous gene. Exemplary “safe harbor” loci include a CCR5 gene, a CXCR4 gene, a PPP1R12C (also known as AAVS1) gene, an albumin gene, and a Rosa gene.

An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule or construct can be the same type of molecule as an endogenous molecule, e.g., an exogenous protein or nucleic acid. In such instances, the exogenous molecule is introduced into the cell at greater concentrations than that of the endogenous molecule in the cell. In some instances, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the expression level of a gene. Modulation of expression can include, but is not limited to, gene activation and gene repression. Modulation may also be complete, i.e. wherein gene expression is totally inactivated or is activated to wildtype levels or beyond; or it may be partial, wherein gene expression is partially reduced, or partially activated to some fraction of wildtype levels.

The term ““operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

“Pluripotent stem cells” as used herein have the potential to differentiate into any of the three germ layers: endoderm (e.g., the stomach lining, gastrointestinal tract, lungs, etc.), mesoderm (e.g., muscle, bone, blood, urogenital tissue, etc.) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al, Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A; HLA-B and HLA-C, which present peptides from the inside of the cell, and antigens presented by the HLA-I complex attract killer T cells (also known as CD8+ T cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ T cells (also known as helper T cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

The terms “treat”, “treating”, “treatment”, etc., as applied to an isolated cell, include subjecting the cell to any kind of process or condition or performing any kind of manipulation or procedure on the cell. As applied to a subject, the terms refer to administering a cell or population of cells in which a target polynucleotide sequence (e.g., B2M) has been altered ex vivo according to the methods described herein to an individual. The individual is usually ill or injured, or at increased risk of becoming ill relative to an average member of the population and in need of such attention, care, or management.

As used herein, the term “treating” and “treatment” refers to administering to a subject an effective amount of cells with target polynucleotide sequences altered ex vivo according to the methods described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a disorder associated with expression of a polynucleotide sequence, as well as those likely to develop such a disorder due to genetic susceptibility or other factors.

By “treatment” or “prevention” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the terms “administering,” “introducing” and “transplanting” are used interchangeably in the context of the placement of cells, e.g., cells described herein comprising a target polynucleotide sequence altered according to the methods of the present technology into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years. In some instances, the cells can also be administered a location other than the desired site, such as in the liver or subcutaneously, for example, in a capsule to maintain the implanted cells at the implant location and avoid migration of the implanted cells.

In additional or alternative aspects, the present technology contemplates altering target polynucleotide sequences in any manner which is available to the skilled artisan, e.g., utilizing a TALEN system. It should be understood that although examples of methods utilizing CRISPR/Cas (e.g., Cas9 and Cas12a) and TALEN are described in detail herein, the present technology is not limited to the use of these methods/systems. Other methods of targeting, e.g., B2M, to reduce or ablate expression in target cells known to the skilled artisan can be utilized herein.

The methods outlined herein can be used to alter a target polynucleotide sequence in a cell. The present technology contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the CRISPR/Cas systems of the present technology. In other instances, a “mutant cell” exhibits a wild-type phenotype, for example when a CRISPR/Cas system of the present technology is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system described herein can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This, in some embodiments, causes increased or decreased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, the alteration results in reduced expression of the target polynucleotide sequence. The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” is intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al, infra).

One example of an algorithm that is suitable for determining percent sequence identity and sequence similarity is the BLAST algorithm, which is described in Altschul et al, J. Mol. Biol. 215:403-410 (1990). Software for performing BLAST analyses is publicly available through the National Center for Biotechnology Information.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present technology. Any recited method may be carried out in the order of events recited or in any other order that is logically possible. Although any methods and materials similar or equivalent to those described herein may also be used in the practice or testing of the technology, representative illustrative methods and materials are now described.

As described in the present technology, the following terms will be employed, and are defined as indicated below.

Before the present technology is further described, it is to be understood that this technology is not limited to particular embodiments described, as such may, of course, vary. It is also to be understood that the terminology used herein is for the purpose of describing particular embodiments only, and is not intended to be limiting, since the scope of the present technology will be limited only by the appended claims.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the technology. The upper and lower limits of these smaller ranges may independently be included in the smaller ranges and are also encompassed within the technology, subject to any specifically excluded limit in the stated range. Where the stated range includes one or both of the limits, ranges excluding either or both of those included limits are also included in the technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the present technology described herein is not entitled to antedate such publication by virtue of prior technology. Further, the dates of publication provided might be different from the actual publication dates, which may need to be independently confirmed.

### III. Detailed Description of the Embodiments

### A. Conditional HIP Cells and Methods for Conditional Downregulation of Immunosuppressive Factors

The introduction of safety switches improves the safety of cell therapies developed using hypoimmunogenic cells (HIP cells). A feature of the HIP cells described herein is the inducible expression of one or more immune regulatory (immunosuppressive) factors In some embodiments, an immunosuppressive factor (also referred to herein as “an hypoimmunity factor”) includes, but is not limited to, CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8. In certain embodiments, the immunosuppressive factor is CD47. The regulatable or inducible expression of an immunosuppressive factor functions to control an immune response by a recipient subject to an engrafted hypoimmunogenic cell.

Described herein are methods for the expression of an immunosuppressive factor that requires a mechanism to ‘turn-off’ expression of the immune regulatory protein in a controlled manner. Also described are HIP cells possessing controllable expression of one or more immunosuppressive factors. In some cases, the cells overexpress one or more immunosuppressive factors and can be induced to downregulate expression of the one or more immunosuppressive factors. As such, the cells are no longer hypoimmunogenic and are recognized by the recipient's immune cells for cell death.

In some embodiments, the hypoimmunity of the cells that are introduced to a recipient subject is achieved through the overexpression of an immunosuppressive molecule including hypoimmunity factors and complement inhibitors accompanied with the repression or genetic disruption of the HLA-I and HLA-II loci. These modifications cloak the cell from the recipient immune system's effector cells that are responsible for the clearance of infected, malignant or non-self cells, such as T cells, B cells, NK cells and macrophages. Cloaking of a cell from the immune system allows for existence and persistence of allogeneic cells within the body. Controlled removal of the engineered cells from the body is crucial for patient safety and can be achieved by uncloaking the cells from the immune system. Uncloaking serves as a safety switch and can be achieved through the downregulation of the immunosuppressive molecules or the upregulation of immune signaling molecules. The level of expression of any of the immunosuppressive molecules described can be controlled on the protein level, mRNA level, or DNA level in the cells. Similarly, the level of expression of any of the immune signaling molecules described can be controlled on the protein level, mRNA level, or DNA level in the cells.

In some embodiments, any of the safety switch methods described (e.g., protein level, RNA level and DNA level safety switches) are used to decrease the level of an immunosuppressive factor in the cells such that the lower level of the immunosuppressive factor is below a threshold level. In some embodiments, the level of the immunosuppressive factor in the cells is decreased by about 10-fold, 9-fold, 8-fold, 7-fold, 6-fold, 5-fold, 4-fold, 3-fold, 2-fold, 1-fold or 0.5-fold below a threshold level of expression. In some embodiments, the level of the immunosuppressive factor in the cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some embodiments, the threshold level of expression of the immunosuppressive factor is established based on the expression of such factor in an induced pluripotent stem cell. In some embodiments, the threshold level of the immunosuppressive factor expression is established based on the expression level of the immunosuppressive factor in a corresponding hypoimmune cell, such as an MHC I and MHC II knockout cell or an MHC I/MHC II/TCR knockout cell.

1. Protein Level Control

In some embodiments, regulated degradation of an immunosuppressive protein is established by incorporating a degron into the amino acid sequence of the immunosuppressive factor that allows recruitment to the endogenous protein turnover machinery. Mechanisms for targeted protein degradation include, but are not limited to, recruitment to an E3 ligase for ubiquitination and subsequent proteasomal degradation, direct recruitment to the proteasome, and recruitment to the lysosome.

Fusion of inducible degron motifs to the immunosuppressive molecules enables exogenous control over the stability of the molecule through the addition or removal of small molecules that stabilize or destabilize the degron, and thus the immunosuppressive molecule.

In some embodiments, methods for inducible protein degradation by a degron includes, but is not limited to, ligand induced degradation (LID) using a SMASH tag, ligand induced degradation using Shield-1, ligand induced degradation using auxin, ligand induced degradation using rapamycin, peptidic degrons (e.g., IKZF3 based degrons), and proteolysis-targeting chimeras (PROTACs). In some embodiments of a ligand induced degradation method, a degron tag that is held in an inactive conformation but is induced to adopt a conformation capable of recognition by the proteasome upon binding of a specific molecule, such as but not limited to, a Shield-1 molecule. See, e.g., Roth et al., Cellular Molecular Life Sciences, 2019, 76(14), 2761-2777, which is herein incorporated by reference in its entirety. Detailed descriptions of SMASH degron technology can be found in Hannah and Zhou, Nat Chem Biol, 2015, 11:637-638 and Chung et al., Nat Chem Biol, 2015, 11:713-720, which are herein incorporated by reference in their entireties. Detailed descriptions of LID degron technologies can be found in Bonger et al., Nat Chem Biol, 2011, 7(8):531-7, which is herein incorporated by reference in its entirety.

In some aspects, provided are methods for controlling the immunogenicity of a mammalian cell (e.g., a human cell) by obtaining an isolated cell and introducing a construct containing a constitutive promoter operably linked to an inducible degron element that is operably linked to a gene encoding an immunosuppressive factor. In some embodiments, the construct includes a constitutive promoter operably linked to an inducible degron element that is operably linked to a nucleic acid sequence encoding flexible linker that is operable linked to a gene encoding an immunosuppressive factor. In some embodiments, the construct comprising a constitutive promoter operably linked to a gene encoding an immunosuppressive factor that is operably linked to an inducible degron element. In some embodiments, the construct includes a constitutive promoter operably linked to a gene encoding an immunosuppressive factor that is linked to a sequence encoding a flexible linker that is operably linked to an inducible degron element. As such, the degron targets the immunosuppressive factor for degradation upon contacting the cell with a degron ligand or molecule.

In some embodiments, the inducible degron element is selected from the group consisting of a ligand inducible degron element such as a small molecule-assisted shutoff (SMASH) degron element, Shield-1 responsive degron element, auxin responsive degron element, and rapamycin responsive degron element; a peptidic degron element; and a peptidic proteolysis targeting chimera (PROTAC) element. In useful embodiments, the ligand inducible degron element is a small molecule-assisted shutoff (SMASH) degron element and the exogenous factor for controlling immunogenicity is asunaprevir. In some embodiments, the immunosuppressive factor gene is selected from the group consisting of CD47, CD24, CD200, HLA-G, HLA-E, HLA-C, HLA-E heavy chain, PD-L1, IDO1, CTLA4-Ig, C1-Inhibitor, IL-10, IL-35, FASL, Serpinb9, CCl21, and Mfge8. In many embodiments, the immunosuppressive factor gene is CD47. In some instances, the constitutive promoter of the construct is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter. In some instances, the optional flexible linker is selected from the group consisting of (GSG)ₙ(SEQ ID NO:3), (GGGS)ₙ (SEQ ID NO:1), and (GGGSGGGS)ₙ (SEQ ID NO:2), wherein n is 1-10. In some embodiments, the construct is introduced into the cell to integrated into a safe harbor locus, such as but not limited to, an AAVS1 locus, a CLBYL locus, a CXCR4 locus, a Rosa26 locus, and a CCR5 locus. In some embodiments, the construct is introduced into the AAVS locus in the cell by way for homology directed recombination. As such, the construct includes 5′ and 3′ homology arms specific to the targeted safe harbor locus. In some embodiments, the construct comprises from 5′ end to 3′ end: a 5′ homology arm to the AAVS1 locus, an exogenous constitutive promoter, an inducible degron element, a gene encoding an immunosuppressive factor, and a 3′ homology arm to the AAVS1 locus. In other embodiments, the construct comprises from 5′ end to 3′ end: a 5′ homology arm to the AAVS1 locus, an exogenous constitutive promoter, an inducible degron element, a sequence encoding flexible linker, a gene encoding an immunosuppressive factor, and a 3′ homology arm to the AAVS1 locus. In useful embodiments, the engineered cell includes an exogenous nucleic acid sequence comprising a constitutive promoter operably linked to an inducible degron element that is operably linked to an optional sequence encoding a flexible linker that is operable linked to a gene encoding an immunosuppressive factor. The engineered cell expresses the inducible degron element fused or linked to an immunosuppressive factor. In some embodiments, the cell is contacted by a factor or agent such as, but not limited to, a ligand, molecule, peptide or small molecule, that activates the degron element to degrade the immunosuppressive factor.

In some embodiments of a peptidic degron, a peptide tag is used that confers small molecule-mediated recruitment to an E3 ligase. In some embodiments, the peptide tag comprises the lymphoid-restricted transcription factor IKZF3 that is recruited to the E3 ligase receptor (CRBN) in an immunomodulatory drug (IMiD) dependent manner, as described in Koduri et al., Proc Natl Acad Sci, 2019, 116(7), 2539-2544, which is herein incorporated by reference in its entirety. In certain embodiments, the degron is capable of targeting immunosuppressive factors for degradation (e.g., through a ubiquitination pathway), inducing protein degradation, or degrading proteins.

In some aspects, provided are methods for controlling the immunogenicity of a mammalian cell (e.g., a human cell) by obtaining an isolated cell and introducing a construct including a constitutive promoter, an inducible peptidic degron element, and a gene encoding an immunosuppressive factor. In some embodiments, the construct includes a constitutive promoter, an inducible peptidic degron element, a nucleic acid sequence encoding flexible linker, and a gene encoding an immunosuppressive factor. Any of the constitutive promoters, immunosuppressive factors, flexible linkers, and cells described herein are applicable to the method.

In some embodiments of a PROTAC, a bifunctional molecule is used to recruit an immunosuppressive factor to the protein degradation machinery of a cell. In some embodiments, the bi-functional molecule binds to the native or wildtype sequence of the immunosuppressive protein or an engineered version of the immunosuppressive protein expressing a domain that binds to the bi-functional molecule with high affinity. In some embodiments, the bi-functional molecule comprises a small molecule or a biologic agent (e.g., an antibody or fragment thereof). See, e.g., Burslem et al., Cell Chemical Biology, 2018, 25, 67-77 and Roth et al., Cellular Molecular Life Sciences, 2019, 76(14), 2761-2777, which are herein incorporated by reference in their entirety.

In some embodiments of a bi-functional antibody, the antibody targets an immunosuppressive factor and a second endogenous receptor which leads to internalization and degradation. Controllable expression of one or more immunosuppressive factors can be provided by way of a bifunctional antibody (e.g., a chemically reprogrammed bifunctional antibody), inducible protein degradation by a degron, inducible RNA regulation, inducible DNA regulation, and an inducible expression method. See, e.g., Natsume and Kanemaki, Annu Rev Genet, 2017, 51, 82-102; Burslem and Crews, Chem Rev, 2017, 117, 11269-11301; Banik et al., ChemRxiv, 2019; which are herein incorporated by reference in their entirety. In some embodiments, a cell expressing an immunosuppressive factor is contacted by an antibody that binds the cell for degradation.

In some instances, hypoimmune cells are availed and cleared by the immune system through the addition of an antibody that binds an epitope on the extracellular surface of the cell. The epitope can be native to the overexpressed immunosuppressive factor, or can be another epitope located within the immunosuppressive factor or distinctly located at the extracellular surface. Binding of an antibody to the surface uncloaks the cell and leads to antibody-dependent cellular cytotoxicity (ADCC) or complement-dependent cytotoxicity (CDC).

In some embodiments, the ADCC/CDC safety switch epitope is selected from the group consisting of EGFR, CD20, CD19, CCR4, HER2, MUC1, GD2, PSMA, CD30, CD16, and fragment, derivative, and variants thereof. In some instances, any of the cells described herein express an epitope selected from an EGFR epitope, CD20 epitope, CD19 epitope. CCR4 epitope. HER2 epitope, MUC1 epitope, GD2 epitope, PSMA epitope, CD30 epitope, or CD16 epitope. In some embodiments, the cells bind to an antibody specific to EGFR, CD20, CD19, CCR4, HER2, MUC1, GD2, PSMA, CD30, or CD16, which leads to ADCC/CDC.

The methods directed to a protein level safety switch as described herein provides a way for decreasing the level of an immunosuppressive factor (e.g., CD47) in an regulatable manner in engineered cells described herein (e.g., hypoimmune cells). By lowering the level of the immunosuppressive factor such as CD47 below a threshold level in the cells using any of the safety switch methods described herein, the recipient subject's immune system can initiate an immune response to such cells. In some embodiments, the level of CD47 in the engineered cells is decreased by the safety switch by about 10-fold, 9-fold, 8-fold, 7-fold, 6-fold, 5-fold, 4-fold, 3-fold, 2-fold, 1-fold or 0.5-fold below a threshold level of expression. In some embodiments, the level of CD47 in the engineered cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some instances, the threshold level of CD47 expression is established based on the exogenous expression of CD47 in an induced pluripotent stem cell. In other instances, the threshold level of CD47 expression is established based on the expression level of CD47 in a corresponding hypoimmune cell, such as an MHC I and MHC II knockout cell or an MHC I/MHC II/TCR knockout cell. In some instances, the level of CD47 is reduced using a degron-based safety switch such as, but not limited to, a SMASH degron or a LID degron. In some embodiments, the cells expressing a SMASH degron linked to an exogenous CD47 transgene are exposed to the small molecule asunaprevir (the degron inducer), which thereby induces a reduction of expression of the exogenous CD47 by the cells.

2. RNA Level Control

Immunosuppressive factors can be targeted by siRNAs or miRNAs, thereby leading to the degradation of the transcript encoding the factors. An siRNA can be exogenously provided or genetically encoded to provide control over transcription of the inhibitory RNA. The siRNA or miRNA can anneal to the immunosuppressive factor's transcript, resulting in degradation by the RISC complex

In some embodiments, methods for inducible RNA regulation to downregulate expression of an immunosuppressive factor include, but are not limited to, shRNAs induced by a small molecule or a biologic agent, inducible siRNAs, inducible miRNAs, inducible CRISPR interference (CRISPRi), and inducible RNA targeting nucleases.

In some embodiments, the method comprises an shRNA or siRNA targeting the RNA of the immunosuppressive factor. In some instances, expression of the shRNA or siRNA is induced by a small molecule or biologic agent.

In some aspects, provided are methods for controlling the immunogenicity of a mammalian cell (e.g., a human cell) by obtaining an isolated cell and introducing a construct containing an inducible RNA polymerase promoter operably linked an shRNA sequence targeting an immunosuppressive factor that is operably linked to a constitutive promoter that is operably linked to a transactivator element that can control the inducible RNA polymerase promoter. In some embodiments, the construct includes a U6Tet promoter, an shRNA targeting an immunosuppressive factor, a constitutive promoter, and a Tet Repressor element that is responsive to tetracycline or a derivative thereof (e.g., doxycycline). In other instances, the shRNA eliminates expression of the immunosuppressive factor. In other instances, the shRNA decreases expression of the immunosuppressive factor by about 99% or less, e.g., 99%, 98%, 97%, 96%, 95%, 94%, 93%, 92%, 90%, 85% or less. In some embodiments, the inducible promoter is a tetracycline responsive promoter. Any of the constitutive promoters, immunosuppressive factors, and cells described herein are applicable to the method.

In many embodiments, the engineered cell expresses an inducible shRNA that targets an immunosuppressive factor. In some embodiments, the cell also expresses an exogenous immunosuppressive factor that mediates the hypoimmunogenicity of the cell. In some embodiments, the cell is contacted by a factor such as, but not limited to, a ligand, molecule, peptide or small molecule, that activates the expression of the shRNA to degrade the immunosuppressive factor.

In some embodiments, the method comprises a CRISPR interference system (CRISPRi) for targeting the promoter of an immunosuppressive factor to downregulate its transcription. In some instances, expression of a CRISPRi and/or a gRNA targeting the immunosuppressive factor is induced by a small molecule or biologic agent. Detailed description of CRISPRi methods are found in, e.g., Engreitz et al., Cold Spring Harb Perspect Biol, 2019, 11:a035386, which is herein incorporated by reference in its entirety. In some embodiments, the CRISPRi system utilizes a dCas9-repressor fusion protein that is controlled by a constitutive promoter and a gRNA specific to the immunosuppressive factor under the control of an inducible promoter.

In some aspects, provided are methods for controlling the immunogenicity of a mammalian cell (e.g., a human cell) by obtaining an isolated cell and introducing into the cell (i) a first construct containing a constitutive promoter operably linked to a gene encoding an immunosuppressive factor; (ii) a second construct containing a constitutive promoter operably linked to a gene encoding a Cas9 nuclease or variant thereof such as dCas9-repressor fusion protein; and (iii) a third construct comprising an inducible RNA polymerase promoter operably linked to a gRNA sequence targeting the sequence encoding the immunosuppressive factor such that the gRNA sequence is operably linked to a transactivator element that corresponds to the inducible RNA polymerase promoter. In some instances, the first construct, second construct, and third construct are found in a single vector. In some instances, the first construct, second construct, and third construct are found in two vectors.

In some embodiments, the CRISPR based method includes a nuclease for targeting the mRNA sequence corresponding to the immunosuppressive factor such as, but not limited to, Cas13, Cas7, or Csx1. In some instances, expression of a nuclease and/or a gRNA targeting the immunosuppressive factor is induced by a small molecule or biologic agent.

In some aspects, provided are methods for controlling the immunogenicity of a mammalian cell (e.g., a human cell) by obtaining an isolated cell and introducing into the cell (i) a first construct comprising a constitutive promoter operably linked to a gene encoding an immunosuppressive factor; (ii) a second construct comprising a constitutive promoter operably linked to a gene encoding a Cas13a nuclease, a variant thereof, or a fusion protein thereof; and (iii) a third construct comprising an inducible RNA polymerase promoter operably linked to a gRNA sequence targeting the sequence encoding the immunosuppressive factor such that the gRNA sequence is operably linked to a transactivator element that corresponds to the inducible RNA polymerase promoter.

In some embodiments, inducible expression systems that are useful for RNA level control of the immunosuppressive factor include, but are not limited to, ligand inducible transcription factor systems, receptor mediated expression control systems, and ligand regulated riboswitches. In some embodiments, the inducible expression system comprises a tetracycline-controlled operator system, a synthetic Notch-based (SynNotch) system (see, e.g., Morsut et al., Cell, 2016, 164:780-791 and Yang et al., Commun Biol, 2020, 3:116), and riboswitch that regulates expression of the immunosuppressive factor gene by ligand (e.g., aptamer, peptide or small molecule) mediated alternative splicing of the resulting pre-mRNA. Useful riboswitches comprise a sensor region and an effector region that sense the presence of a ligand and alter the splice of the target immunosuppressive factor gene. Detailed descriptions and examples of riboswitch gRNAs are found in e.g., U.S. Pat. Nos. 9,228,207; 9,993,491; and 10,421,989; and Seeliger et al., PLoS One, 2012, 7(1):e29266; the contents are herein incorporated by reference in their entirety.

In some embodiments, the level of an immunosuppressive factor such as CD47 in the engineered cells is decreased by an RNA level safety switch by about 10-fold, 9-fold, 8-fold, 7-fold, 6-fold, 5-fold, 4-fold, 3-fold, 2-fold, 1-fold or 0.5-fold below a threshold level of expression. In some embodiments, the level of CD47 in the engineered cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some instances, the threshold level of CD47 expression is established based on the exogenous expression of CD47 in an induced pluripotent stem cell. In other instances, the threshold level of CD47 expression is established based on the expression level of CD47 in a corresponding hypoimmune cell, such as an MHC I and MHC II knockout cell or an MHC I/MHC II/TCR knockout cell.

3. DNA Level Control

Transcriptional regulation of immunosuppressive factors through employing inducible promoters provides the ability to turn expression of the switch on or off at will through the addition or removal of small molecules, such as, but not limited to, doxycycline. Genetic disruption via targeted nuclease activity can eliminate expression of the immunosuppressive factor to uncloak the cells as well.

In some embodiments, methods for inducible DNA regulation include, but are not limited to, using tissue-specific promoters, inducible promoters, controllable riboswitches, and knockout using an inducible nuclease (e.g., inducible CRISPRs, inducible TALENs, inducible zinc finger nucleases, inducible homing endonucleases, inducible meganucleases, and the like) to target the DNA sequence of one or more immunosuppressive factors. In some embodiments, the inducible nuclease comprises a nuclease such that its expression is controlled by the presence of a small molecule. In some embodiments, the inducible nuclease comprises a nuclease such that delivery of the nuclease RNA or protein to a cells is controlled by the presence of a small molecule. In some embodiments, expression of the nuclease is induced by a small molecule or biologic agent. In some embodiments, expression of a Cas nuclease and/or a guide RNA (gRNA) is induced by a small molecule or biologic agent.

In some embodiments, methods for inducible expression include, but are not limited to, ligand inducible transcription factors systems (e.g., a tetracycline-controlled operator system), receptor mediated control of expression system (e.g., a SynNotch system), and a ligand regulated riboswitch system for control of mRNA or gRNA activity. Detailed description of inducible expression methods are found in, e.g., Kallunki et al., Cells, 2019, 796 (doi:10.3390/ce11s8080796), which is herein incorporated by reference in its entirety.

In some embodiments, the immunosuppressive factors are expressed in a cell using an inducible expression vector. The expression vector can be a viral vector, such as but not limited to, a lentiviral vector. In some embodiments, the inducible immunosuppressive factors described herein are introduced into a cell by lentiviral transduction.

In some embodiments, the silencing of a construct encoding the immunosuppressive factor results in elimination of the engineered cell by a recipient subject's immune system. Furthermore, the construct containing the immunosuppressive factor and an inducible expression system can be integrated into an endogenous gene locus to safeguard expression of the cassette, as silencing of the gene will eliminate the engineered cells. In some embodiments, the endogenous gene locus useful for integration is a core essential gene locus or an immune signaling factor gene locus. Non-limiting examples of a core essential gene locus for such integration include RpS2, RpS9, RpS11, RpS13, RpS18, RpL8, RpL11, RpL32, RpL36, Rpn11, Psmd14, and PSMA3. Non-limiting examples of an immune signaling factor gene locus for such integration include B2M, MIC-A/B, HLA-A, HLA-B, HLA-C, RFXANK; CTLA4, PD1, and ligands of NKG2D (e.g., MICA, MICB, RAET1E/ULBP4, RAET1G/ULBP5, RAET1H/ULBP2, RAET1/ULBP1, RAET1L/ULBP6, and RAET1N/ULBP3).

In an exemplary embodiment, the conditional expression of an immunosuppressive factor is based on regulating expression of the immune regulatory factor CD47. CD47 is a component of the innate immune system that functions as a “do not eat me” signal as part of the innate immune system to block phagocytosis by macrophages. Useful immunosuppressive factors that can be engineered for controlled expression include, but are not limited to, CD47, CD27, CD200, HLA-C, HLA-E, HLA-E heavy chain, HLA-G, PD-L1, ID01, CTLA4-Ig, C1-Inhibitor, IL-10, IL-35, FASL, Serpinb9, CCL21, and Mfge8.

In some embodiments, the present disclosure provides a method of producing a stem cell (e.g., hypoimmunogenic pluripotent stem cell or hypoimmunogenic induced pluripotent stem cell) or a differentiated cell thereof that has been modified to conditionally express any one of the immunosuppressive factors selected from the group consisting of CD47, CD27; CD200, HLA-C, HLA-E, HLA-E heavy chain, HLA-G, PD-L1, ID01, CTLA4-Ig, C1-Inhibitor, IL-10, IL-35, FASL, Serpinb9, CCL21, and Mfge8. In other embodiments, the immunosuppressive factor is selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, and HELIOS.

In some embodiments, the cells conditionally express one or more of the immunosuppressive factors such that in the absence of the exogenous controlling signal, the cells are hypoimmunogenic or have reduced hypoimmunogenicity. In the presence of the exogenous controlling signal, the cells are recognized by immune cells and are targeted by cell death or clearance. In some instances, the HIP cells express an immunosuppressive factor that functions allow the HIP cell to evade the recipient subject's immune response. Upon exposing the HIP cells to an exogenous controlling signal, the expression (e.g., the DNA level expression, the RNA level expression, or the protein level expression) of immunosuppressive factor is downregulated; and thus the HIP cells are recognized by the innate immune system in the recipient subject. As such, the HIP cells undergo cell death and/or cell clearance in the recipient.

In some embodiments, the level of an immunosuppressive factor such as CD47 in the engineered cells is decreased by a DNA level safety switch by about 10-fold, 9-fold, 8-fold, 7-fold, 6-fold, 5-fold, 4-fold, 3-fold, 2-fold, 1-fold or 0.5-fold below a threshold level of expression. In some embodiments, the level of CD47 in the engineered cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some instances, the threshold level of CD47 expression is established based on the exogenous expression of CD47 in an induced pluripotent stem cell. In other instances, the threshold level of CD47 expression is established based on the expression level of CD47 in a corresponding hypoimmune cell, such as an MHC I and MHC II knockout cell or an MHC I/MHC II/TCR knockout cell.

### B. Conditional HIP Cells and Methods Conditional Upregulation of Immune Signaling Factors

Described herein are methods for the expression of an immune signaling factor in a controllable manner as to increase the expression of the factor to alter the hypoimmunogenicity of the cell. Also described are HIP cells that possess controllable expression of one or more immune signaling factors. In some aspects, the immune signaling factor is selected from the group consisting of B2M, MIC-A/B, HLA-A, HLA-B, HLA-C, RFXANK, CTLA-4, PD-1, and ligands of NKG2D (e.g., MICA, MICB, RAET1E/ULBP4, RAET1G/ULBP5, RAET1H/ULBP2, RAET1/ULBP1, RAET1L/ULBP6, and RAET1N/ULBP3).

Controllable expression of one or more immune signaling factors can be provided by way of a inducible ligand stabilization system using a degron, an inducible RNA upregulation system (e.g., an inducible CRISPR activation), and an inducible DNA upregulation system. In some embodiments, the inducible DNA upregulation system comprises inducible CRISPR activation (CRISPRa), tissue-specific promoters, inducible promoters, and riboswitches.

Detailed description of CRISPRa methods are found in, e.g., Engreitz et al., Cold Spring Harb Perspect Biol, 2019, 11:a035386, which is herein incorporated by reference in its entirety. Detailed descriptions and examples of inducible riboswitches are found in e.g., U.S. Pat. Nos. 9,228,207; 9,993,491; and 10,421,989; and Seeliger et al., PLoS One, 2012, 7(1):e29266; the contents are herein incorporated by reference in their entirety.

### C. Bicistronic Constructs for Expressing Safety Switches and Target Factors

Described herein is a system for associating the expression of a safety switch to the expression of a target factor (e.g., a hypoimmunity factor or an essential cell factor) in cells, thereby ensuring clearance of cells with silenced expression of the safety switch. By placing the safety switch 5′ to the gene encoding a target factor (e.g., a hypoimmunity factor or an essential cell factor), a silencing event or mutation that disrupts expression of the safety switch will also disrupt expression of the target factor (e.g., a hypoimmunity factor or an essential cell factor), thereby making the mutated cells non-viable and undergo apoptosis.

A key component of a bicistronic construct of the present technology is a safety switch that kills a cell containing the construct, in the presence of a drug or a prodrug. In some embodiments, the disclosure provides hypoimmunogenic cells (e.g., HIP stem cells or differentiated cells thereof) that comprise a “suicide gene” (or “suicide switch”). The suicide gene is incorporated to function as a “safety switch” that can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites.

Provided herein are bicistronic constructs for the coexpression of a safety switch (e.g., a safety protein) and a target factor such as a hypoimmunity factor or an essential cell factor. In some embodiments, coexpression of a safety switch and hypoimmune molecule is obtained through the expression of a polycistronic transcript, whereby the target factor and safety switch are separated by a ribosomal skipping sequence. In some embodiments, the expression of the construct (e.g., cassette) is regulated either by a promoter in the case of genomic location-independent transcriptional regulation or by a splice acceptor to enable regulation of the payload (e.g., the safety switch and the target factor) by an endogenous promoter following integration of the construct into a selected target gene.

In some embodiments, the safety switch transgene of the construct is selected from the group consisting of a HSVtk gene, cytosine deaminase gene, nitroreductase gene, purine nucleoside phosphorylase gene, horseradish peroxidase gene, iCaspase9 gene, HER1 transgene, RQR8 transgene, CD20 transgene, CCR4 transgene, CD19 transgene, MUC1 transgene, EGFR transgene, HER2 transgene, GD2 transgene, PSMA transgene, CD16 transgene, and CD30 transgene. In some embodiments, the HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, or CD30 transgene comprise an epitope thereof. In some instances, the transgene comprises a gene encoding an epitope selected from a group consisting of a CD20 epitope, CCR4 epitope, CD19 epitope, MUC1 epitope, EGFR epitope, HER2 epitope, GD2 epitope, PSMA epitope, CD16 epitope, and CD30 epitope. In some embodiments, the transgene comprises an epitope that binds to a CD20 gene product is recognized by an anti-CD20 therapeutic antibody selected from the group consisting of obinutuzumab, ublituximab, ocaratuzumab, rituximab, rituximab-RLIb, and biosimilars thereof; an anti-CCR4 therapeutic antibody selected from the group consisting of mogamulizumab and biosimilars thereof; an anti-HER2 therapeutic antibody selected from the group consisting of margetuximab, trastuzumab, TrasGEX, and biosimilars thereof; an anti-CD19 therapeutic antibody selected from the group consisting of MOR208 and biosimilars thereof, an anti-MUC1 therapeutic antibody selected from the group consisting of gatipotuzumab and biosimilars thereof; an anti-EGFR therapeutic antibody selected from the group consisting of tomuzotuximab, RO5083945 (GA201), cetuximab, and biosimilars thereof; an anti-GD2 therapeutic antibody selected from the group consisting of Hu14.18K322A, Hu14.18-1L2, Hu3F8, dinituximab, c.60C3-RLIc, and biosimilars thereof; an anti-PSMA therapeutic antibody selected from the group consisting of KM2812 and biosimilars thereof; an anti-CD30 or anti-CD16 therapeutic antibody selected from the group consisting of AFM13 and biosimilars thereof, or an anti-CD20 or anti-CD16 therapeutic antibody selected from the group consisting of (CD20)2×CD16 and biosimilars thereof.

Descriptions of safety switches and uses thereof are described in Dusgunes, N. (2019) Origins of Suicide Gene Therapy. In: Düzgüneş N. (eds) Suicide Gene Therapy. Methods in Molecular Biology, vol 1895. Humana Press, New York, N.Y. (for HSVtk, cytosine deaminase, nitroreductase, purine nucleoside phosphorylase, and horseradish peroxidase); Zhou and Brenner, Exp Hematol, 2016, 44(11):1013-1019 (for iCaspase9); Wang et al., Blood, 2001, 18(5), 1255-1263 (for huEGFR); US20180002397 (for HER1); and Philip et al., Blood, 2014, 124(8), 1277-1287 (for RQR8).

In some instances, the thymidylate synthase gene or a mutant thereof is included in the construct. For example, cells expressing thymidylate synthase are sensitive to certain prodrugs including ganciclovir. Expression of thymidylate synthase within the cell renders the cell sensitive to the prodrug ganciclovir. In another embodiment, the CD20 gene is included. Cells that are CD20-positive can be killed through treatment with an anti-CD20 antibody (e.g., rituximab or a biosimilar or surrogate thereof) In some cases, the HSVtk transgene is controlled by the exogenous factor ganciclovir. In some cases, the cytosine deaminase transgene is controlled by the exogenous factor 5-fluorocytosine. In some cases, the nitroreductase transgene is controlled by the exogenous factor CB1954. In some cases, the purine nucleoside phosphorylase transgene is controlled by the exogenous factor 6-methylpurine deoxyriboside or fludarabine. In some cases, the horseradish peroxidase transgene is controlled by the exogenous factor indole3-acetic acid.

In some cases, the iCaspase9 transgene is controlled by the exogenous factor rimiducid (AP1903), AP20187, or rapamycin. In some cases, the human truncated EGFR transgene (e.g., EGFRt) is controlled by the exogenous antibody cetuximab or a variant thereof that recognizes the same or similar epitope. In some cases, the human HER1 transgene is controlled by the exogenous antibody cetuximab or a variant thereof that recognizes the same or similar epitope. In some cases, the human RQR8 transgene is controlled by the exogenous antibody rituximab or a variant thereof that recognizes the same or similar epitope.

In some embodiments, the CD20 gene product is recognized by a therapeutic antibody selected from the group consisting of obinutuzumab, ublituximab, ocaratuzumab, rituximab, rituximab-RLIb, and biosimilars thereof; the CCR4 gene product is recognized by a therapeutic antibody selected from the group consisting of mogamulizumab and biosimilars thereof; the HER2 gene product is recognized by a therapeutic antibody selected from the group consisting of margetuximab, trastuzumab, TrasGEX, and biosimilars thereof; the CD19 gene product is recognized by a therapeutic antibody selected from the group consisting of MOR208 and biosimilars thereof; the MUC1 gene product is recognized by a therapeutic antibody selected from the group consisting of gatipotuzumab and biosimilars thereof; the EGFR gene product is recognized by a therapeutic antibody selected from the group consisting of tomuzotuximab, RO5083945 (GA201), cetuximab, and biosimilars thereof; the GD2 gene product is recognized by a therapeutic antibody selected from the group consisting of Hu14.18K322A, Hu14.18-1L2, Hu3F8, dinituximab, c.60C3-RLIc, and biosimilars thereof; the PSMA gene product is recognized by a therapeutic antibody selected from the group consisting of KM2812 and biosimilars thereof; the CD30 or CD16 gene product is recognized by a therapeutic antibody selected from the group consisting of AFM13 and biosimilars thereof, or the CD20 or CD16 gene product is recognized by a therapeutic antibody selected from the group consisting of (CD20)2×CD16 and biosimilars thereof.

In some embodiments, the safety switch transgene is an inducible Caspase protein. An inducible Caspase protein includes at least a portion of a Caspase protein capable of inducing apoptosis. In many embodiments, the inducible Caspase protein is iCasp9. In some instances, iCasp9 includes the sequence of the human FK506-binding protein, FKBP12, with an F36V mutation, connected through a series of amino acids to the gene encoding human caspase 9. FKBP12-F36V binds with high affinity to a small-molecule dimerizing agent, rimiducid or AP1903. Thus, the suicide function of iCasp9 is triggered by the administration of a chemical inducer of dimerization (CID). In some embodiments, the CID is the small molecule drug AP1903. Dimerization causes the rapid induction of apoptosis. See, e.g., WO2011146862; Stasi et al, N. Engl. J. Med 365; 18 (2011); Tey et al, Biol. Blood Marrow Transplant. 13:913-924 (2007), each of which are incorporated by reference herein in their entirety.

In some embodiments, the safety switch transgene is an antibody-dependent cell-mediated cytoxicity (ADCC) and complement-dependent cytoxicity (CDC) dependent safety switch. In some instances, the safety switch transgene comprises an EGFR fragment or epitope, a CD20 fragment or epitope, or a CD19 fragment or epitope.

In some cases, the human EGFR safety switch is controlled by the antibody cetuximab, a variant thereof that recognizes the same or similar epitope, or another anti-EGFR antibody. In some cases, the human CD19 safety switch is controlled by the antibody bevacizumab, a variant thereof that recognizes the same or similar epitope, or another anti-CD19 antibody. In some cases, the human CD20 safety switch is controlled by the antibody rituximab, a variant thereof that recognizes the same or similar epitope, or another anti-CD20 antibody.

In some embodiments, a safety switch is coexpressed with a hypoimmunity factor selected from the group consisting of CD47, CD24, CD200, HLA-G, HLA-E, HLA-C, HLA-E heavy chain, PD-L1, IDO1, CTLA4-Ig, IL-10, IL-35, FASL, Serpinb9, CCl21, and Mfge8. In certain embodiments, the hypoimmunity factor is CD47.

In some instances, the bicistronic construct also includes a natural or synthetic terminator. For example, encompassed for use in the presently disclosed constructs include any one of the terminators identified and described in Chen et al., Nature Methods, 2013, 10, 659-664, the contents of which are herein incorporated by reference. In some embodiments, the terminator is located at the 3′ end of the expression construct. In some embodiments, the terminator is operably linked to a target factor gene (e.g., a hypoimmunity factor gene or an essential cell factor gene).

In some instances, the bicistronic construct includes a ribosomal skipping sequence such as, but not limited to, a sequence encoding an IRES sequence or a sequence encoding a 2A-coding sequence. Non-limiting examples of self-cleaving 2A-coding sequence include T2A, P2A, E2A, and F2A. Exemplary sequences of the T2A, P2A, E2A, and F2A peptides are shown in Table 2.

In some instances, the bicistronic construct includes a linker, e.g., a peptide linker, flexible linker, and the like, located between the safety switch and the target factor. Exemplary linkers are provided in Table 2.

In some embodiments, the bicistronic construct includes a transcriptional regulatory element. In some instances, the transcriptional regulatory element controls expression of the safety switch and the hypoimmunity factor. In some embodiments, the transcriptional regulatory element is a promoter or a splice acceptor. In some embodiments, the promoter is a constitutive promoter selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter. Exemplary sequences of constitutive promoters is provided in Table 4.

In some embodiments, the bicistronic construct is designed for lentiviral expression. Provided herein are lentiviral vectors comprising the bicistronic construct as outlined. A useful lentiviral vector backbone is selected according to the cell types to be transduced.

Provided herein are bicistronic constructs that include a safety switch transgene operably linked to a ribosomal skipping sequence and/or a sequence encoding a linker, which is operably linked a gene encoding a target factor (e.g., a hypoimmunity factor or an essential cell factor). For example, it is noted that positioning the safety switch 5′ to the hypoimmunity factor gene in a bicistronic format ensures that a silencing event such as a frame-shift mutation that inactivates the safety switch will also inactivate the hypoimmunity factor gene. In some embodiments, expression of the bicistronic constructs is regulated by a constitutive promoter that is operably linked to the safety switch transgene operably linked to the ribosomal skipping sequence and/or the sequence encoding the linker, which is operably linked the hypoimmunity factor gene. In some embodiments, the construct also includes polyadenylation sequence at the 3′ end. In some embodiments, the construct includes a natural or synthetic terminator.

Also provided are bicistronic constructs that include a hypoimmunity factor gene (or an essential cell factor gene) operably linked to a ribosomal skipping sequence or a linker which is operably linked a safety switch transgene. In some instances, a frameshift mutation in the safety-switch does not inactivate the hypoimmunity factor (or essential cell factor) in this bicistronic format. In some embodiments, expression of the bicistronic constructs is regulated by a constitutive promoter that is operably linked the target factor gene operably linked to the ribosomal skipping sequence or the linker which is operably linked the safety switch transgene. In some embodiments, the construct also includes polyadenylation sequence at the 3′ end. In some embodiments, the construct includes a natural or synthetic terminator.

For all of these technologies, well known recombinant techniques are used, to generate recombinant nucleic acids as outlined herein. In certain embodiments, the recombinant nucleic acids encoding any of the factors described herein may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus long terminal repeat region, and the early promoter of human cytomegalovirus (CMV). Examples of other heterologous mammalian promoters includes the actin, immunoglobulin or heat shock promoter(s). In additional embodiments, promoters for use in mammalian cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). The early and late promoters of SV40 are conveniently obtained as an SV40 restriction fragment which also contains the SV40 viral origin of replication (Fiers et al, Nature 273: 113-120 (1978)). The immediate early promoter of the human cytomegalovirus is conveniently obtained as a HindIII restriction enzyme fragment (Greenaway et al, Gene 18: 355-360 (1982)). The foregoing references are incorporated by reference in their entirety.

The process of introducing the polynucleotides described herein into cells can be achieved by any suitable technique. Suitable techniques include, but are not limited to, calcium phosphate or lipid-mediated transfection, electroporation, and transduction or infection using a viral vector. In some embodiments, the polynucleotides are introduced into a cell via viral transduction (e.g., lentiviral transduction). Once altered, the presence of expression of any of the molecule described herein can be assayed using known techniques, such as Western blots, ELISA assays, FACS assays, and the like.

In some embodiments, the constructs described herein are introduced into isolated cells such as isolated mammalian cells and isolated human cells. In some embodiments, the cells are stem cells, embryonic stem cells, pluripotent stem cells, induced pluripotent stem cells, adult stem cell, or differentiated cells thereof. In some instances, the cells are hypoimmunogenic. Hypoimmunogenic cells and methods of generating such are described herein.

### D. Homology Directed Repair (HDR) of Safety Switches and Target Factors

In some aspects, a construct designed for coexpression of the safety switch (e.g., a degron and the like) and a target factor (e.g., an immunosuppressive factor or an essential cell factor) as a single mRNA transcript is introduced at an endogenous locus by homology directed repair (HDR). In this configuration, the inserted elements disrupt the endogenous coding sequence. In some embodiments, the endogenous locus is an essential cell factor gene locus. In such cases, the introduced tandem construct expressed under control of the endogenous promoter compensates for deletion of the essential cell factor gene and creates codependence on expression of the safety switch. In some instances, knocking in of a safety switch into an essential gene allows for evasion of expression pressure.

In some embodiments, the construct containing a promoter and a bicistronic expression construct is introduced by HDR at a genomic locus such as a safe harbor locus, an immune signaling gene locus, or an essential cell factor gene locus followed by bi-allelic knock-out of the endogenous essential cell factor gene (or in some cases, a gene encoding an immunosuppressive factor) using a targeted nuclease. In this configuration, silent mutations in the sequence encoding the essential cell factor gene are introduced in the bicistronic construct to confer resistance to nuclease cleavage. The introduced tandem expression construct compensates for deletion of the essential cell factor gene and creates co-dependence on expression of the safety switch.

In some embodiments, the construct for HDR into a safe harbor locus comprises: a first homology arm homologous to a first endogenous sequence of a safe harbor locus; a safety switch transgene; a ribosomal skipping sequence and/or a sequence encoding a linker; an immunosuppressive factor gene (or an essential cell gene); a polyadenylation sequence; and a second homology arm homologous to a second endogenous sequence of the safe harbor locus. In some embodiments, the construct for HDR into an immune signaling gene locus comprises: a first homology arm homologous to a first endogenous sequence of an immune signaling gene locus; a safety switch transgene; a ribosomal skipping sequence and/or a sequence encoding a linker; an immunosuppressive factor gene; a polyadenylation sequence; and a second homology arm homologous to a second endogenous sequence of the immune signaling gene locus. In some embodiments, the construct for HDR into an essential cell factor gene locus comprises: a first homology arm homologous to a first endogenous sequence of an essential cell factor gene locus; a safety switch transgene; a ribosomal skipping sequence and/or a sequence encoding a linker; an immunosuppressive factor gene; a polyadenylation sequence; and a second homology arm homologous to a second endogenous sequence of the essential cell factor gene locus. In some instances, a transcriptional regulatory element is located 5′ of the safety switch transgene.

In some embodiments, the transcriptional regulatory element is selected from the group consisting of an EF1A promoter, an EFS promoter, a CMV promoter, a CAGGS promoter (also known as the CAG promoter), an SV40 promoter, a COPIA promoter, an ACT5C promoter, a TRE promoter, a CBh promoter, a PGK promoter, and a UBC promoter.

In some embodiments, the safety switch transgene of the construct is selected from the group consisting of a HSVtk gene, cytosine deaminase gene, nitroreductase gene, a purine nucleoside phosphorylase gene, horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

As described above, the immunosuppressive factor can be, but is not limited to, CD47, CD24, CD200, HLA-G, HLA-E, HLA-C, HLA-E heavy chain, PD-L1, ID01, CTLA4-Ig, IL-10, IL-35, FASL, Serpinb9, CCl21, and Mfge8. In some instances, the essential cell factor can be, but is not limited to, RpS2, RpS9, RpS11, RpS13, RpS18, RpL8, RpL11, RpL32, RpL36, Rpn22, Psmd14, PSMA3, a ribosome subunit, a proteasome subunit, and a spliceosome subunit. In some embodiments, an immunosuppressive factor such as CD47 along with a safety switch transgene are introduced into a gene encoding an essential cell factor such that silencing of the essential cell factor gene is mitigated. In some cases, the gene encodes an essential cell factor selected from the group consisting of the proteasome subunit Psmd14, the ribosomal subunit Rps2, and the ribosomal subunit RpL32.

In some embodiments, the linker is a peptide linker, flexible linker, and the like, located between the safety switch and the hypoimmunity factor. In some embodiments, the linker is a peptide linker, flexible linker, and the like, located between the safety switch and the essential cell factor. Exemplary linkers are provided in Table 2.

In some embodiments, the ribosomal skipping sequence comprises a sequence encoding an IRES sequence or a sequence encoding a 2A-coding sequence. Non-limiting examples of self-cleaving 2A-coding sequence include T2A, P2A, E2A, and F2A. Exemplary sequences of the T2A, P2A, E2A, and F2A peptides are shown in Table 2.

In some embodiments for targeted integration, the safe harbor locus is selected from the group consisting of an AAVS1 locus, a CLBYL locus, a CXCR4 locus, a Rosa26 locus, and a CCR5 locus. In many embodiments, the safe harbor locus is a CLBYL locus or a CCR5 locus. In some embodiments, the immune signaling gene locus is selected from the group consisting of B2M, HLA-A, HLA-B, HLA-C, HLA-D, HLA-E, RFXANK, CIITA, CTLA4, PD1, and ligands of NKG2D (e.g., MICA, MICB, RAET1E/ULBP4, RAET1G/ULBP5, RAET1H/ULBP2, RAET1/ULBP1, RAET1L/ULBP6, and RAET1N/ULBP3). In some embodiments, the essential cell factor locus is selected from the group consisting of a RpS2 gene locus. RpS9 gene locus, RpS11 gene locus, RpS13 gene locus, RpS18 gene locus, RpL8 gene locus, RpL11 gene locus, RpL32 gene locus, RpL36 gene locus, Rpn22 gene locus, Psmd14 gene locus, PSMA3 gene locus, a gene locus for a ribosome subunit, a gene locus for a proteasome subunit, and a gene locus for a spliceosome subunit.

Targeted integration of the safety switch and the immunosuppressive factor into the selected locus can be accomplished using targeted nuclease technology such as CRISPR-based and non-CRISPR-based methods described herein.

Outlined herein are cells expressing the safety switch and the immunosuppressive factor in a safe harbor locus. Also provided are cells expressing the safety switch and the immunosuppressive factor in an immune signaling gene locus. Expression of the safety switch is associated with the expression of the immunosuppressive factor within viable cells. In some embodiments, the cells are mammalian cells and isolated human cells. In some embodiments, the cells are stem cells, embryonic stem cells, pluripotent stem cells, induced pluripotent stem cells, adult stem cell, or differentiated cells thereof. In some instances, the cells are hypoimmunogenic. Hypoimmunogenic cells and methods of generating such are described herein.

In some aspects, provided is a homology independent donor construct comprising from 5′ to 3′ end: a 5′ long terminal repeats (LTR) comprising a left element (LE); a splice acceptor-viral 2A peptide (SA-2A) element; a safety switch transgene; a ribosomal skipping sequence or a sequence encoding a linker; an immunosuppressive factor gene; a polyadenylation sequence; and 3′ LTR comprising a right element (RE). In some embodiments, the construct is introduced into a cell by way of Cas9-induced HDR. In some embodiments, the construct is introduced into a cell by way of homology independent integration.

Also provided is a homology independent donor construct comprising from 5′ to 3′ end: a 5′ long terminal repeats (LTR) comprising a left element (LE); a splice acceptor-viral 2A peptide (SA-2A) element; a hypoimmunity factor gene; a ribosomal skipping sequence or a sequence encoding a linker; a safety switch transgene; a polyadenylation sequence; and 3′ LTR comprising a right element (RE). In some embodiments, the construct is introduced into a cell by way of Cas9-induced HDR. In some embodiments, the construct is introduced into a cell by way of homology independent integration.

In some embodiments, any one of the constructs described are introduced into isolated cells such as isolated mammalian cells and isolated human cells. In some embodiments, the cells are stem cells, embryonic stem cells, pluripotent stem cells, induced pluripotent stem cells, adult stem cell, or differentiated cells thereof. In some instances, the cells are hypoimmunogenic. Hypoimmunogenic cells and methods of generating such are described herein.

### E. Hypoimmunity Factor-Safety Switch Fusion Proteins

ADCC and CDC function via immune effector cells by recognizing antibodies bound to the extracellular surface of a cell. ADCC/CDC is activated by expression of an epitope recognized by the antibodies. As such, this system can act as an effective safety switch. Provided herein is a fusion protein comprising an epitope and a hypoimmune molecule. The fusion protein provides a fail-safe for eliminating engineered hypoimmune cells. Peptidic epitopes, such as the CD20 fragment recognized by rituximab (referred to as “a CD20 mimotope”) are linked to extracellular or membrane-bound hypoimmune molecules such as, but not limited to, CD47.

In some embodiments, the fusion protein comprises a hypoimmunity factor and a peptidic epitope. In some embodiments, the fusion protein comprises a hypoimmunity factor, a peptidic epitope, and a linker.

In some embodiments, the fusion protein comprises from N- to C-terminal: a hypoimmunity factor and a peptidic epitope. In some embodiments, the fusion protein comprises from N- to C-terminal: a peptidic epitope and a hypoimmunity factor. In some embodiments, the fusion protein comprises from N- to C-terminal: a hypoimmunity factor; a linker; and a peptidic epitope. In some embodiments, the fusion protein comprises from N- to C-terminal: a peptidic epitope; a linker; and a hypoimmunity factor.

In some embodiments, the fusion protein comprises from N- to C-terminal: a linker; a hypoimmunity factor; a linker; and a peptidic epitope. In some embodiments, the fusion protein comprises from N- to C-terminal: a hypoimmunity factor; a linker; a peptidic epitope; and a linker. In some embodiments, the fusion protein comprises from N- to C-terminal: a linker; a peptidic epitope; a linker; and a hypoimmunity factor, and a linker.

In one embodiment, the fusion protein comprises from N- to C-terminal: a surface-exposed human CD20 epitope and a hypoimmunity factor. In some embodiments, the fusion protein comprises from N- to C-terminal: a surface-exposed human CD20 epitope, a linker, and a hypoimmunity factor. In some embodiments, the fusion protein comprises from N- to C-terminal: a linker, a surface-exposed CD20 epitope, a linker, and a hypoimmunity factor. In some embodiments, the fusion protein comprises from N- to C-terminal: a surface-exposed CD20 epitope, a linker, a hypoimmunity factor, and a linker. In particular embodiments, the fusion protein comprises from N- to C-terminal: a hypoimmunity factor and a surface-exposed human CD20 epitope. In some embodiments, the fusion protein comprises from N- to C-terminal: a hypoimmunity factor, a linker, and a surface-exposed human CD20 epitope. In some embodiments, the fusion protein comprises from N- to C-terminal: a linker, a hypoimmunity factor, a linker, and a surface-exposed human CD20 epitope. In some embodiments, the fusion protein comprises from N- to C-terminal: a hypoimmunity factor, a linker, a surface-exposed human CD20 epitope, and a linker.

In some embodiments, the fusion protein comprises from N- to C-terminal: an optional linker; a human CD20 epitope; an optional linker; and a hypoimmunity factor. In some embodiments, the human CD20 epitope is recognized by rituximab, a variant thereof, or another anti-CD20 antibody. In some embodiments, the fusion protein comprises from N- to C-terminal: an optional linker; a human CD19 epitope; an optional linker; and a hypoimmunity factor. In some embodiments, the human CD19 epitope is recognized by bevacizumab, a variant thereof, or another anti-CD19 antibody. In some embodiments, the fusion protein comprises from N- to C-terminal: an optional linker; a human EGFR epitope; an optional linker; and a hypoimmunity factor. In some embodiments, the human EGFR epitope is recognized by cetuximab, a variant thereof, or another anti-EGFR antibody. In some embodiments, the fusion protein comprises from N- to C-terminal: an optional linker; a human CCR4 epitope; an optional linker; and a hypoimmunity factor. In some embodiments, the human CCR4 epitope is recognized by an anti-CCR4 antibody. In some embodiments, the fusion protein comprises from N- to C-terminal: an optional linker; a human MUC1 epitope; an optional linker; and a hypoimmunity factor. In some embodiments, the human MUC1 epitope is recognized by an anti-MUC1 antibody. In some embodiments, the fusion protein comprises from N- to C-terminal: an optional linker; a human CD16 epitope or a human CD30 epitope; an optional linker; and a hypoimmunity factor. In some embodiments, the human CD30 epitope is recognized by an anti-CD30 antibody or a bispecific antibody thereof. In some embodiments, the human CD16 epitope is recognized by an anti-CD16 antibody or a bispecific antibody thereof. In some embodiments, the fusion protein comprises from N- to C-terminal: an optional linker; a human CD20 epitope or a human CD16 epitope; an optional linker; and a hypoimmunity factor. In some embodiments, the human CD20 epitope is recognized by an anti-CD20 antibody or a bispecific antibody thereof. In some embodiments, the human CD16 epitope is recognized by an anti-CD16 antibody or a bispecific antibody thereof. In some embodiments, the fusion protein comprises from N- to C-terminal: an optional linker; a human PSMA epitope; an optional linker; and a hypoimmunity factor. In some embodiments, the human PSMA epitope is recognized by an anti-PSMA antibody. In some embodiments, the fusion protein comprises from N- to C-terminal: an optional linker; a human GD2 epitope; an optional linker; and a hypoimmunity factor. In some embodiments, the human GD2 epitope is recognized by an anti-GD2 antibody. In other embodiments, the order of the peptide epitope and the hypoimmunity factor are reversed.

In other embodiments, the fusion protein comprises from N- to C-terminal: a human CD47 fragment comprising the IgV domain of CD47; a linker; a peptidic epitope; a linker; and a human CD47 transmembrane domain.

In another aspect, provided herein is a bicistronic construct comprising from 5′ to 3′ end: a transcriptional regulatory element; a sequence encoding a peptidic epitope; a ribosomal skipping sequence; and a sequence encoding a hypoimmunity factor. In some embodiments, the peptidic epitope is selected from the group consisting of CD20 epitope, CCR4 epitope, CD19 epitope; MUC1 epitope, EGFR epitope, HER2 epitope, GD2 epitope, PSMA epitope, CD16 epitope, and CD30 epitope.

In some embodiments, the hypoimmunity factor is selected from the group consisting of CD47, CD24, CD200, HLA-G, HLA-E, HLA-C, HLA-E heavy chain, PD-L1, IDO1, CTLA4-Ig, IL-10, IL-35, FASL, Serpinb9, CCl21, Mfge8, and membrane-bound forms thereof. In certain embodiments, the hypoimmunity factor is CD47. In some embodiments, a linker is selected from one in Table 2.

In some embodiments, any one of the peptide epitopes is selected from the group consisting of a CD20 epitope, CCR4 epitope, CD19 epitope, MUC1 epitope, EGFR epitope, HER2 epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof. In some embodiments, the CCR4 epitope is recognized by a therapeutic antibody selected from the group consisting of mogamulizumab and biosimilars thereof. In some embodiments, the HER2 epitope is recognized by a therapeutic antibody selected from the group consisting of margetuximab, trastuzumab, TrasGEX, and biosimilars thereof. In some embodiments, the CD19 epitope is recognized by a therapeutic antibody selected from the group consisting of MOR208 and biosimilars thereof. In some embodiments, the MUC1 epitope is recognized by a therapeutic antibody selected from the group consisting of gatipotuzumab and biosimilars thereof. In some embodiments, the EGFR epitope is recognized by a therapeutic antibody selected from the group consisting of tomuzotuximab, RO5083945 (GA201), cetuximab, and biosimilars thereof. In some embodiments, the GD2 epitope is recognized by a therapeutic antibody selected from the group consisting of Hu14.18K322A, Hu14.18-IL2, Hu3F8, dinituximab, c.60C3-RLIc, and biosimilars thereof. In some embodiments, the PSMA epitope is recognized by a therapeutic antibody selected from the group consisting of KM2812 and biosimilars thereof. In some embodiments, the CD30 or CD16 epitope is recognized by a therapeutic antibody selected from the group consisting of AFM13 and biosimilars thereof. In some embodiments, the CD20 or CD16 epitope is recognized by a therapeutic antibody selected from the group consisting of (CD20)2×CD16 and biosimilars thereof.

Any one of the constructs described can be introduced into isolated cells such as isolated mammalian cells and isolated human cells. In some embodiments, the cells are stem cells, embryonic stem cells, pluripotent stem cells, adult stem cell, or differentiated cells thereof. In some instances, the cells are hypoimmunogenic. Hypoimmunogenic cells and methods of generating such are described herein.

### F. Conditional HIP Cells with Modified Expression of MHC I, MHC II and TCR Complexes

In one aspect, the present technology disclosed herein is directed the use of safety switch to regulate expression of target factors in pluripotent stem cells, (e.g., pluripotent stem cells and induced pluripotent stem cells (iPSCs)), differentiated cells derived from such pluripotent stem cells (e.g., hypoimmune T cells), and primary T cells. In certain embodiments, the pluripotent stem cells, differentiated cells derived therefrom, and primary T cells are engineered for reduced expression or deleted expression of MHC class I and MHC class II human leukocyte antigens. In certain embodiments, the pluripotent stem cells, differentiated cells derived therefrom, and primary T cells are engineered for reduced expression or deleted expression of MHC class I and MHC class II human leukocyte antigens, and reduced expression or deleted expression of one or more T cell receptor (TCR) complexes. In some instances, deleted or reduced expression of MHC class I antigens, MHC class II antigens, and/or one or more TCR complexes is achieved using an inducible gene modification system.

In some aspects, the present disclosure provides pluripotent stem cells, (e.g., pluripotent stem cells and induced pluripotent stem cells (iPSCs)), differentiated cells derived from such pluripotent stem cells (e.g., hypoimmune T cells), primary T cells, and populations thereof comprising a genome in which a gene has been edited to delete a contiguous stretch of genomic DNA, thereby reducing or eliminating surface expression of MHC class I molecules in the cells or a population thereof. In certain aspects, the present disclosure provides pluripotent stem cells, (e.g., pluripotent stem cells and induced pluripotent stem cells (iPSCs)), differentiated cells derived from such pluripotent stem cells (e.g., hypoimmune T cells), primary T cells, and populations thereof comprising a genome in which a gene has been edited to delete a contiguous stretch of genomic DNA, thereby reducing or eliminating surface expression of MHC class II molecules in the cells or a population thereof. In particular aspects, the present disclosure provides pluripotent stem cells, (e.g., pluripotent stem cells and induced pluripotent stem cells (iPSCs)), differentiated cells derived from such pluripotent stem cells (e.g., hypoimmune T cells), primary T cells, and populations thereof comprising a genome in which one or more genes has been edited to delete a contiguous stretch of genomic DNA, thereby reducing or eliminating surface expression of MHC class I and II molecules in the cells or a population thereof. In further aspects, the present disclosure provides pluripotent stem cells, (e.g., pluripotent stem cells and induced pluripotent stem cells (iPSCs)), differentiated cells derived from such pluripotent stem cells (e.g., hypoimmune T cells), primary T cells, and populations thereof comprising a genome in which one or more genes has been edited to delete a contiguous stretch of genomic DNA, thereby reducing or eliminating surface expression of one or more TCR complexes in the cells or a population thereof.

In some embodiments, the cells include a genomic modification of one or more targeted polynucleotide sequences that regulates the expression of MHC I and/or MHC II. In some aspects, a genetic editing system is used to modify one or more targeted polynucleotide sequences. In some embodiments, the targeted polynucleotide sequence is one or more selected from the group consisting of B2M, CIITA, and NLRC5. In certain embodiments, the genome of the cell has been altered to reduce or delete critical components of HLA expression. In additional embodiments, the cells include a genomic modification of one or more targeted polynucleotide sequences that regulates the expression of one or more TCR complexes. In some aspects, a genetic editing system is used to modify one or more targeted polynucleotide sequences. In some embodiments, the targeted polynucleotide sequence is one or more selected from the group consisting of TRAC and TRB.

In some embodiments, the cells and methods described herein include genomically editing human cells to cleave CIITA gene sequences as well as editing the genome of such cells to alter one or more additional target polynucleotide sequences such as, but not limited to, B2M, NLRC5, TRAC, and TRB. In some embodiments, the cells and methods described herein include genomically editing human cells to cleave B2M gene sequences as well as editing the genome of such cells to alter one or more additional target polynucleotide sequences such as, but not limited to, CIITA, NLRC5, TRAC, and TRB. In some embodiments, the cells and methods described herein include genomically editing human cells to cleave NLRC5 gene sequences as well as editing the genome of such cells to alter one or more additional target polynucleotide sequences such as, but not limited to, B2M, CIITA, TRAC and TRB. In some embodiments, the cells and methods described herein include genomically editing human cells to cleave TRAC gene sequences as well as editing the genome of such cells to alter one or more additional target polynucleotide sequences such as, but not limited to, B2M, CIITA, NLRC5 and TRB. In some embodiments, the cells and methods described herein include genomically editing human cells to cleave TRB gene sequences as well as editing the genome of such cells to alter one or more additional target polynucleotide sequences such as, but not limited to, B2M, CIITA, NLRC5 and TRAC.

In some embodiments, pluripotent stem cells, differentiated cells derived from such, and primary T cells include a genomic modification of the B2M gene. In some embodiments, pluripotent stem cells, differentiated cells derived from such, and primary T cells include a genomic modification of the CIITA gene. In some embodiments, pluripotent stem cells, differentiated cells derived from such, and primary T cells include a genomic modification of the TRAC gene. In some embodiments pluripotent stem cells, differentiated cells derived from such, and primary T cells include a genomic modification of the TRB gene. In some embodiments, pluripotent stem cells, differentiated cells derived from such, and primary T cells include genomic modifications of the B2M and CIITA. In some embodiments, pluripotent stem cells, differentiated cells derived from such, and primary T cells include one or more genomic modifications selected from the group consisting of the B2M, CIITA and TRAC genes. In some embodiments, pluripotent stem cells, differentiated cells derived from such, and primary T cells include one or more genomic modifications selected from the group consisting of the B2M, CIITA and TRB genes. In some embodiments, pluripotent stem cells, differentiated cells derived from such, and primary T cells include one or more genomic modifications selected from the group consisting of the B2M, CIITA, TRAC and TRB genes. In some embodiments, the cells are B2M^{-/-}, CIITA^{-/-} cells. In many embodiments, the cells are B2M^{-/-}, CIITA^{-/-}, TRAC^{-/-} cells. In many embodiments, the cells are B2M^{-/-}, CIITA^{-/-}, TRB^{-/-} cells. In some embodiments, the cells are B2M^{indel/indel}, CIITA^{indel/indel} cells. In some embodiments, the cells are B2M^{indel/indel}, CIITA^{indel/indel}, TRAc^{indel/indel} cells. In some embodiments, the cells are B2M^{indel/indel}, CIITA^{indel/indel}, TRB^{indel/indel} cells. In some embodiments, the cells are B2M^{indel/indel}, CIITA^{indel/indel}, TRAc^{indel/indel}, TRB^{indel/indel} cells. In some embodiments, the modified cells described are pluripotent stem cells, induced pluripotent stem cells, cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells.

In some embodiments, one or more genes selected from the group consisting of B2M, HLA-A, HLA-B, HLA-C, HLA-D, HLA-E, HLA-DP, HLA-DM, HLA-DOA, HLA-DOB, HLA-DQ, and HLA-DR are inactivated in the cells. The genes can be inactivated using homology dependent repair or site-site specific nuclease. In some embodiments, one or both alleles of the gene are inactivated.

1. CIITA

In certain aspects; the present technology modulates (e.g., reduce or eliminate) the expression of MHC II genes by targeting and modulating (e.g., reducing or eliminating) Class II transactivator (CIITA) expression. In some aspects, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present technology is a variant of CIITA. In some embodiments, the target polynucleotide sequence is a homolog of CIITA. In some embodiments, the target polynucleotide sequence is an ortholog of CIITA.

In some aspects, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In certain cases, the amino acid sequence of CIITA is depicted as NCBI GenBank No. AAA88861.1. Additional descriptions of the CIITA protein and gene locus can be found in Uniprot No. P33076, HGNC Ref. No. 7067, and OMIM Ref. No. 600005.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Table 12 of WO2016183041, which is herein incorporated by reference. In some embodiments, the cell has a reduced ability to induce an immune response in a recipient subject.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

2. B2M

In certain embodiments, the technologies disclosed herein modulate (e.g., reduce or eliminate) the expression of MHC-I genes by targeting and modulating (e.g., reducing or eliminating) expression of the accessory chain B2M. In some aspects, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an immune response in a recipient subject.

In some embodiments, the target polynucleotide sequence of the present technology is a variant of B2M. In some embodiments, the target polynucleotide sequence is a homolog of B2M. In some embodiments, the target polynucleotide sequence is an ortholog of B2M.

In some aspects, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016183041, which is herein incorporated by reference.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

3. NLRC5

In certain aspects, the present technologies modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some aspects, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence of the present technology is a variant of NLRC5. In some embodiments, the target polynucleotide sequence is a homolog of NLRC5. In some embodiments, the target polynucleotide sequence is an ortholog of NLRC5.

In some aspects, decreased or eliminated expression of NLRC5 reduces or eliminates expression of one or more of the following MHC I molecules—HLA-A, HLA-B, and HLA-C.

In some embodiments, the hypoimmunogenic cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 (Table 14 of WO2016183041) provided herewith. In some embodiments, the cell has a reduced ability to induce an immune response in a recipient subject.

Assays to test whether the NLRC5 gene has been inactivated are known and described herein. In one embodiment, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

4. TRAC

In certain embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some aspects, the modulation occurs using a CRISPR/Cas system. By modulating (e.g., reducing or deleting) expression of TRAC, surface trafficking of TCR molecules is blocked. In some embodiments, the cell also has a reduced ability to induce an immune response in a recipient subject.

In some embodiments, the target polynucleotide sequence of the present technology is a variant of TRAC. In some embodiments, the target polynucleotide sequence is a homolog of TRAC. In some embodiments, the target polynucleotide sequence is an ortholog of TRAC.

In some aspects, decreased or eliminated expression of TRAC reduces or eliminates TCR surface expression.

In some embodiments, the cells described herein comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in RefSeq. No. NG_001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS:532-609 and 9102-9797 of US20160348073, which is herein incorporated by reference.

Assays to test whether the TRAC gene has been inactivated are known and described herein. In one embodiment, the resulting genetic modification of the TRAC gene by PCR and the reduction of TCR expression can be assays by FACS analysis. In another embodiment, TRAC protein expression is detected using a Western blot of cells lysates probed with antibodies to the TRAC protein. In another embodiment, reverse transcriptase polymerase chain reactions (RT-PCR) are used to confirm the presence of the inactivating genetic modification.

5. TRB

In certain embodiments, the technologies disclosed herein modulate (e.g., reduce or eliminate) the expression of TCR genes including the gene encoding T cell antigen receptor, beta chain (e.g., the TRB or TCRB gene) by targeting and modulating (e.g., reducing or eliminating) expression of the constant region of the T cell receptor beta chain. In some aspects, the modulation occurs using a CRISPR/Cas system. By modulating (e.g., reducing or deleting) expression of TRB, surface trafficking of TCR molecules is blocked. In some embodiments, the cell also has a reduced ability to induce an immune response in a recipient subject.

In some embodiments, the target polynucleotide sequence of the present technology is a variant of TRB. In some embodiments, the target polynucleotide sequence is a homolog of TRB. In some embodiments, the target polynucleotide sequence is an ortholog of TRB.

In some aspects, decreased or eliminated expression of TRB reduces or eliminates TCR surface expression.

In some embodiments, the cells described herein comprise gene modifications at the gene locus encoding the TRB protein. In other words, the cells comprise a genetic modification at the TRB locus. In some instances, the nucleotide sequence encoding the TRB protein is set forth in UniProt No. PODSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. PODSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS:610-765 and 9798-10532 of U520160348073, which is herein incorporated by reference.

Assays to test whether the TRB gene has been inactivated are known and described herein. In one embodiment, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, reverse transcriptase polymerase chain reactions (RT-PCR) are used to confirm the presence of the inactivating genetic modification.

### G. Methods of Reducing or Eliminating MHC class I, MHC Class II and/or TCR Expression

Provided herein are methods for modifying or engineering cells with reduced expression of MHC I antigens, MHC II antigens, and/or one or more TCR complexes. Reduction of MHC I and/or MHC II expression can be accomplished, for example, by one or more of the following: (1) targeting the polymorphic HLA alleles (HLA-A, HLA-B, HLA-C) and genes directly; (2) removal of B2M, which will prevent surface trafficking of all MHC-I molecules; (3) removal of CIITA, which will prevent surface trafficking of all MHC-II molecules; and/or (4) deletion of components of the MHC enhanceosomes, such as LRC5, RFX-5, RFXANK, RFXAP, IRF1, NF-Y (including NFY-A, NFY-B, NFY-C), and CIITA that are critical for HLA expression.

In some aspects, HLA expression is interfered with by targeting individual HLAs (e.g., knocking out expression of HLA-A, HLA-B, HLA-C, HLA-DP, HLA-DQ, and/or HLA-DR), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of MHC class I molecules (e.g., knocking out expression of B2M and/or TAP1), and/or targeting with HLA-Razor (see, e.g., WO2016183041).

In certain aspects, the cells disclosed herein including, but not limited to, pluripotent stem cells, induced pluripotent stem cells, differentiated cells derived from such stem cells, and primary T cells do not express one or more human leukocyte antigens (e.g., HLA-A, HLA-B, HLA-C, HLA-DP, HLA-DQ, and/or HLA-DR) corresponding to MHC-I and/or MHC-II and are thus characterized as being hypoimmunogenic. For example, in certain aspects, the pluripotent stem cells and induced pluripotent stem cells disclosed have been modified such that the stem cell or a differentiated stem cell prepared therefrom do not express or exhibit reduced expression of one or more of the following MHC-I molecules: HLA-A, HLA-B and HLA-C. In some aspects, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, gRNAs that allow simultaneous deletion of all MHC class I alleles by targeting a conserved region in the HLA genes are identified as HLA Razors. In some aspects, the gRNAs are part of a CRISPR system. In alternative aspects, the gRNAs are part of a TALEN system. In one aspect, an HLA Razor targeting an identified conserved region in HLAs is described in WO2016183041. In other aspects, multiple HLA Razors targeting identified conserved regions are utilized. It is generally understood that any guide that targets a conserved region in HLAs can act as an HLA Razor.

Methods provided below are useful for inactivation or ablation of MHC class I expression, MHC class II expression, and/or TCR expression in cells such, as but not limited to, pluripotent stem cells, pluripotent stem cells, differentiated cells thereof, and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in human stem cells. In certain embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated MHC I and MHC II expression. In some embodiments, the cells are nonimmunogenic (e.g., do not induce an immune response) in a recipient subject.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The present disclosure contemplates altering target polynucleotide sequences in any manner which is available to the skilled artisan utilizing a CRISPR/Cas system. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems described herein can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, the CRISPR/Cas system includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cash, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Y pest subtype (also known as CASS3). Exemplary Cas proteins of the Y pest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6.

In some embodiments, a Cas protein comprises any one of the Cas proteins described herein or a functional portion thereof. As used herein, “functional portion” or “function fragment” refers to a portion of a peptide or protein factor which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein.

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present technology contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present technology can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid.

Exemplary gRNA sequences useful for CRISPR/Cas-based targeting of genes described herein are provided in Table 1. The sequences can be found in WO2016/183041 filed May 9, 2016 and US2016/0348073 filed Mar. 28, 2016, the disclosure of which including the Tables, Appendices, and Sequence Listing are incorporated herein by reference in their entireties.

TABLE 1
Exemplary gRNA sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |
| Gene Name | SEQ ID NO: | US20160348073 |
| TRAC | SEQ ID NOS: 532-609 and | |
| | 9102-9797 | |
| TRB (also | SEQ ID NOS: 610-765 and | |
| TCRB) | 9798-10532 | |

In some aspects, the cells are modified using non-CRISPR based methods. In some embodiments, such methods include, but are not limited to, Transcription Activator-Like Effector Nucleases (TALENs), zinc finger nuclease (ZFNs) homing endonucleases, sequence-specific endonucleases, meganuclease, RNA silencing or RNA interference, and RNA guided transposases.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-nuclease. A monomeric TALE-nuclease is a TALE-nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term “zinc finger binding protein” is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

In some embodiments, the cells of the present disclosure are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the present disclosure may for example correspond to a LAGLIDADG homing endonuclease, to a HNH endonuclease, or to a GIY-YIG endonuclease. In some cases, the homing endonuclease is an I-CreI variant.

In some embodiments, the cells outlined herein are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells described are made using RNA silencing or RNA interference (RNAi) to knockdown (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting RNAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, and NLRC5.

In some embodiments, RNA guided transposases are utilized to integrate DNA into the genome of a cell described herein. Detailed descriptions of useful RNA guided transposases and methods of use thereof are disclosed, e.g., in Klompe et al., Nature 571, 219-225 (2019) and Strecker et al., Science 365, 48-53 (2019), the contents of which are herein incorporated by reference.

### H. Generation of Hypoimmunogenic Pluripotent Stem Cells

The disclosure provides methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPSCs. The original induction was done from mouse embryonic or adult fibroblasts using the viral introduction of four transcription factors, Oct3/4, Sox2; c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### I. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

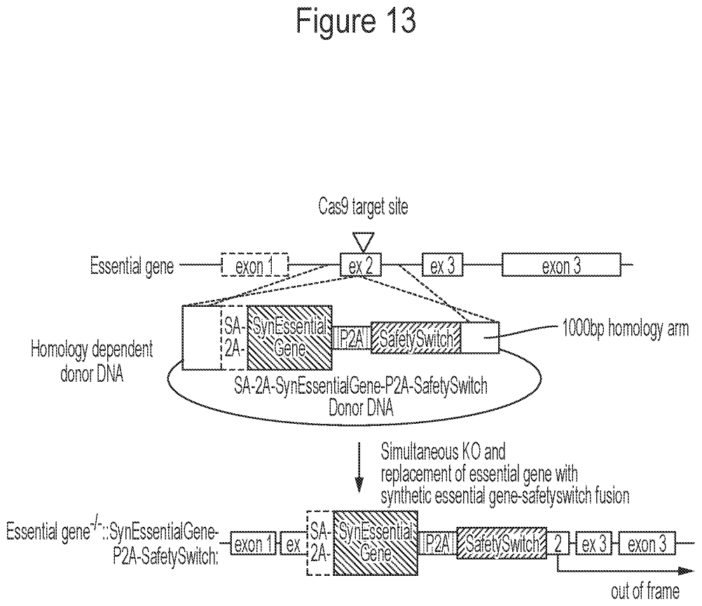

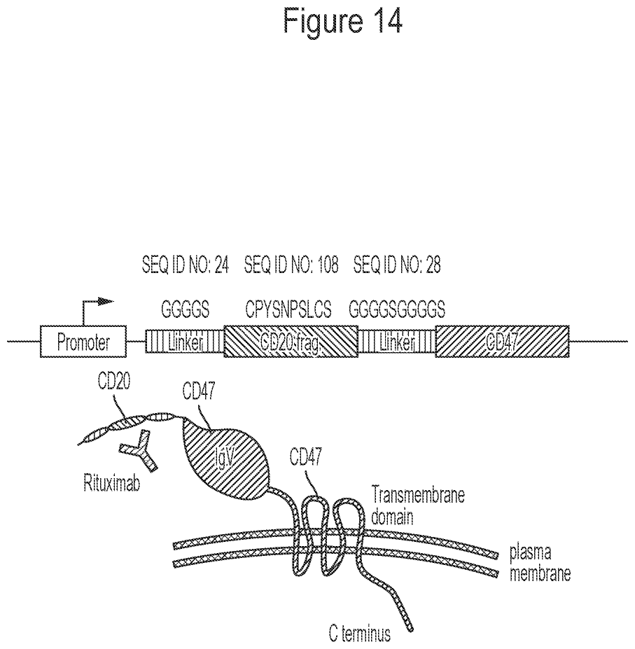

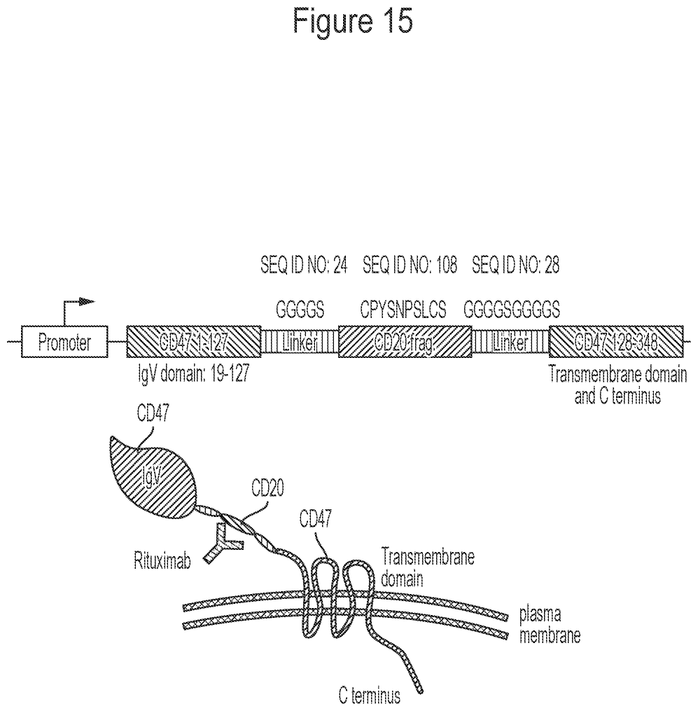

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g. teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell function is assessed by ELISpot, ELISA, FACS, PCR, or mass cytometry (CYTOF). B cell response or antibody response is assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

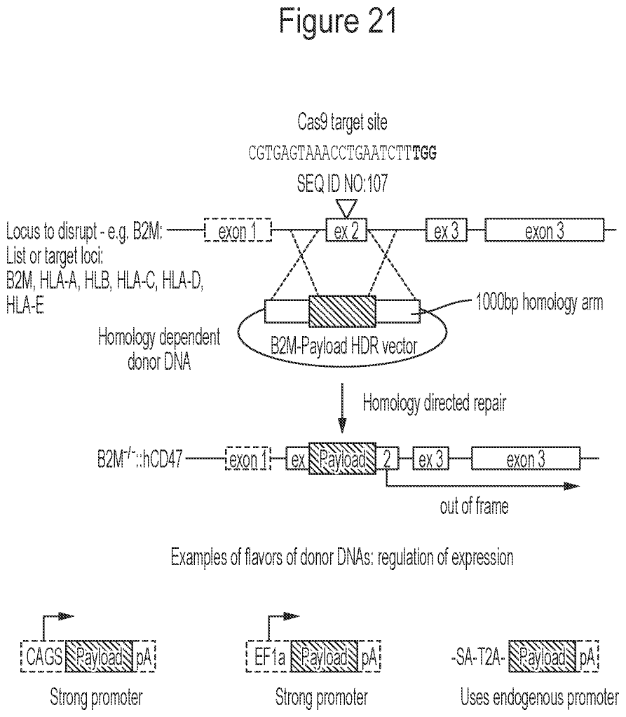

In addition, the cells can be tested to confirm that the HLA II complex is not expressed on the cell surface. Again, this assay is done as is known in the art (See FIG. 21 of WO2018132783, for example) and generally is done using either Western blotting or FACS analysis based on commercial antibodies that bind to human HLA Class II HLA-DR, DP and most DQ antigens.

In addition to the reduction of HLA I and II (or MHC I and II), the hypoimmunogenic cells outlined herein have a reduced susceptibility to macrophage phagocytosis and NK cell killing. The resulting hypoimmunogenic cells “escape” the immune macrophage and innate pathways due to the expression of one or more CD47 transgenes.

### J. Maintenance of Hypoimmunogenic Pluripotent Stem Cells

Once the hypoimmunogenic pluripotent stem cells have been generated, they can be maintained an undifferentiated state as is known for maintaining iPSCs. For example, the cells can be cultured on matrigel using culture media that prevents differentiation and maintains pluripotency. In addition, they can be in culture medium under conditions to maintain pluripotency.

### K. Differentiation of Hypoimmunogenic Pluripotent Stem Cells

The present technology provides hypoimmunogenic pluripotent cells that are differentiated into different cell types for subsequent transplantation into subjects. As will be appreciated by those in the art, the methods for differentiation depend on the desired cell type using known techniques. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the hypoimmunogenic pluripotent cells are differentiated into hepatocytes to address loss of the hepatocyte functioning or cirrhosis of the liver. There are a number of techniques that can be used to differentiate hypoimmunogenic pluripotent cells into hepatocytes; see for example Pettinato et al., doi:10.1038/spre32888, Snykers et al., Methods Mol Biol 698:305-314 (2011), Si-Tayeb et al, Hepatology 51:297-305 (2010) and Asgari et al., Stem Cell Rev (:493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the hypoimmunogenic pluripotent cells are differentiated into pancreatic beta-like cells or islet organoids for transplantation to address type I diabetes mellitus (T1DM). Cell systems are a promising way to address T1DM, see, e.g., Ellis et al., doi/10.1038/nrgastro.2017.93, incorporated herein by reference. Additionally, Pagliuca et al. reports on the successful differentiation of β-cells from human iPSCs (see doi/10.106/j.cell.2014.09.040, hereby incorporated by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; (doi:10.1038/nm.4030, hereby incorporated by reference in its entirety and in particular for the methods and reagents outlined there for the large-scale production of functional human β cells from human pluripotent stem cells).

Differentiation can be assayed as is known in the art, generally by evaluating the presence of β cell associated or specific markers, including but not limited to, insulin. Differentiation can also be measured functionally, such as measuring glucose metabolism, see generally Muraro et al, doi:10.1016/j.cels.2016.09.002, hereby incorporated by reference in its entirety, and specifically for the biomarkers outlined there.

In some embodiments, the hypoimmunogenic pluripotent cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi:10.1056/NEJMoa1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See, for example, Kamao et al., doi:10.1016/j.stemcr.2013.12.007, hereby incorporated by reference in its entirety and specifically for the markers outlined in the first paragraph of the results section.

In some embodiments, the hypoimmunogenic pluripotent cells are differentiated into cardiomyocytes to address cardiovascular diseases. Techniques are known in the art for the differentiation of hiPSCs to cardiomyocytes. Differentiation can be assayed as is known in the art, generally by evaluating the presence of cardiomyocyte associated or specific markers or by measuring functionally; see for example Loh et al., doi:10.1016/j.cell.2016.06.001, hereby incorporated by reference in its entirety and specifically for the methods of differentiating stem cells including cardiomyocytes.

In some embodiments, the hypoimmunogenic pluripotent cells are differentiated into endothelial colony forming cells (ECFCs) to form new blood vessels to address peripheral arterial disease. Techniques to differentiate endothelial cells are known. See, e.g., Prasain et al., doi:10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the hypoimmunogenic pluripotent cells are differentiated into thyroid progenitor cells and thyroid follicular organoids that can secrete thyroid hormones to address autoimmune thyroiditis. Techniques to differentiate thyroid cells are known the art. See, e.g. Kurmann et al., doi:10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446.

### L. Administration of Differentiated Hypoimmunogenic Cells

As will be appreciated by those in the art, the differentiated hypoimmunogenic pluripotent cell derivatives can be transplanted using techniques known in the art that depends on both the cell type and the ultimate use of these cells. In general, the cells of the outlined can be transplanted either intravenously or by injection at particular locations in the patient. When transplanted at particular locations, the cells may be suspended in a gel matrix to prevent dispersion while they take hold.

In some embodiments, provided herein is a method of treating a patient in need of cell therapy comprising administering a population of differentiated cells comprising a differentiated cell generated from a stem cell conditionally expressing an exogenous immunosuppressive factor. In useful embodiments, provided herein is a method of treating a patient in need of cell therapy includes administering a population of differentiated cells comprising a differentiated cell generated from a stem cell conditionally expressing exogenous human CD47.

In some embodiments, the method of treating a patient in need of cell therapy includes administering a population of differentiated cells comprising a differentiated cell generated from a stem cell conditionally expressing a hypoimmunity factor. In many embodiments, the differentiated cell is generated from a stem cell conditionally expression an essential factor.

As will be appreciated by those in the art, the differentiated hypoimmunogenic pluripotent cell derivatives can be transplanted using techniques known in the art that depends on both the cell type and the ultimate use of these cells. In general, the cells outlined herein can be transplanted either intravenously or by injection at particular locations in the patient. When transplanted at particular locations, the cells may be suspended in a gel matrix to prevent dispersion while they take hold.

### M. Exemplary Embodiments

1. Safety Switches for Regulation of Immunosuppressive Factors

In some aspect, provided herein is a method for controlling the immunogenicity of a cell comprising: (a) obtaining an isolated cell; (b) introducing into the isolated cell (i) a nucleic acid comprising an inducible RNA polymerase promoter operably linked to an shRNA sequence targeting an immunosuppressive factor; and (ii) a nucleic acid comprising a promoter (e.g., a constitutive promoter) operably linked to a transactivator element corresponding to the inducible RNA polymerase promoter to produce an engineered cell; and (c) exposing the engineered cell to an exogenous factor to activate the transactivator element, thereby controlling the immunogenicity of the cell. In some embodiments, the method further comprises administering the engineered cell to a subject prior to step (c).

In some embodiments, the method includes introducing into the isolated cell a single construct comprising (i) an inducible RNA polymerase promoter operably linked to an shRNA sequence targeting an immunosuppressive factor and (ii) a promoter (e.g., a constitutive promoter) operably linked to a transactivator element corresponding to an inducible RNA polymerase promoter. In some embodiments, the construct comprises from 5′ end to 3′ end an inducible RNA polymerase promoter; an shRNA sequence targeting an immunosuppressive factor; a promoter (e.g., a constitutive promoter); and a transactivator element.

In some embodiments, a first construct comprises the nucleic acid comprising an inducible RNA polymerase promoter operably linked to an shRNA sequence targeting an immunosuppressive factor, and a second construct comprises the nucleic acid comprising a promoter (e.g., a constitutive promoter) operably linked to a transactivator element.

In some embodiments, the isolated cell is engineered to exogenously express the immunosuppressive factor. In some embodiments, the isolated cell overexpresses the immunosuppressive factor in the absence of the exogenous factor that activates the transactivator element.

In some embodiments, the inducible RNA polymerase promoter of the construct is a U6Tet promoter.

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8. In some embodiments, the immunosuppressive factor is CD47.

In some embodiments, the promoter described above is a constitutive promoter. In some embodiments, the constitutive promoter of the construct is selected from the group consisting of an eukaryotic translation elongation factor 1 al (EF1A) promoter, an eukaryotic translation elongation factor 1 al short form (EFS) promoter, a cytomegalovirus immediate-early enhancer/promoter (CMV promoter), a CMV early enhancer fused to modified chicken β-actin (CAGGS) promoter (also referred to as a CAG promoter), an Simian virus 40 (SV40) promoter, a copia transposon (COPIA) promoter, an actin 5C (ACT5C) promoter, a tetracycline-responsive promoter element (TRE promoter), a CMV early enhancer fused to modified chicken β-actin (CBh) promoter, a phosphoglycerate kinase 1 (PGK) promoter, and a ubiquitin C (UBC) promoter.

In certain embodiments, the construct comprises from 5′ end to 3′ end: a U6Tet promoter, an shRNA sequence targeting CD47, an EF1a promoter, and a Tet Repressor element, and wherein the exogenous factor is tetracycline or a derivative thereof.

In some embodiments, any one of the constructs outlined herein further comprises a vector backbone for lentiviral expression.

In some embodiments, the isolated cell is an isolated mammalian cell. In some embodiments, the isolated cell is an isolated human cell.

In some embodiments, the isolated human cell described above further comprises deletion or reduced expression of MHC class I human leukocyte antigens compared to an unmodified human cell. In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments, the isolated human cell further comprises deletion or reduced expression of B2M. In some embodiments, the isolated human cell further comprises deletion or reduced expression of NLRC5. In some embodiments, the isolated human cell is hypoimmunogenic.

In some embodiments, the isolated human cell is selected from the group consisting of a stem cell, embryonic stem cell, pluripotent stem cell, adult stem cell, and a differentiated cell. In some embodiments, the differentiated cell is selected from the group consisting of a cardiac cell, liver cell, kidney cell, pancreatic cell, neural cell, immune cell, mesenchymal cell, and endothelial cell.

In another aspect, provided herein is a construct comprising from 5′ end to 3′ end: an inducible RNA polymerase promoter; an shRNA sequence targeting an immunosuppressive factor; a constitutive promoter; and a transactivator element corresponding to the inducible RNA polymerase promoter. In some embodiments of the construct, the inducible RNA polymerase promoter is a U6Tet promoter.

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8. In some embodiments, the immunosuppressive factor is CD47.

In some embodiments, the constitutive promoter of the construct is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the construct comprises from 5′ end to 3′ end: a U6Tet promoter, an shRNA sequence targeting CD47, an EF1a promoter, and a Tet Repressor element.

In some embodiments, the nucleic acid or construct also comprises a vector backbone for lentiviral expression.

Also provided herein is a composition comprising an isolated cell comprising any one of the constructs described. In some embodiments of the composition, the isolated cell is exposed to an exogenous factor to activate the transactivator element. In some embodiments, the isolated cell described above is engineered to exogenously express the immunosuppressive factor. In certain embodiments, the isolated cell overexpresses the immunosuppressive factor in the absence of the exogenous factor that activates the transactivator element.

In some embodiments, the isolated cell is selected from the group consisting of a stem cell, embryonic stem cell, pluripotent stem cell, and adult stem cell.

Also provided is a composition comprising isolated differentiated cells prepared by culturing any stem cell described herein under differentiation conditions to produce a differentiated cell. In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

Provided herein is a method of treating a patient in need of cell therapy comprising: (a) administering any one of the compositions described to a patient; and (b) exposing the composition to an exogenous factor to activate the inducible RNA polymerase promoter, thereby controlling immunogenicity of the cells of the composition.

In some aspect, provided herein is a method for controlling the immunogenicity of a cell comprising: (a) obtaining an isolated cell; (b) introducing into the isolated cell a nucleic acid encoding an inducible degron element operably linked to an immunosuppressive factor to produce an engineered cell, or a nucleic acid encoding an immunosuppressive factor operably linked to an inducible degron element; and (c) exposing the engineered cell to an exogenous factor to activate the inducible degron element, thereby controlling the immunogenicity of the cell.

In some embodiments, the method further comprises administering the engineered cell into a subject prior to step (c). In some embodiments, the inducible degron element is linked to the immunosuppressive factor by a flexible linker.

In some embodiments, the inducible degron element is N-terminal to the immunosuppressive factor. In some embodiments, the inducible degron element is C-terminal to the immunosuppressive factor. In some embodiments, transcription of the nucleic acid described is regulated by a promoter such as a constitutive promoter. In some embodiments, provided herein is a construct comprising the nucleic acid outlined above.

In some embodiments, the constitutive promoter in the construct is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the flexible linker is selected from the group consisting of (GSG)ₙ(SEQ ID NO:3), (GGGS)ₙ (SEQ ID NO:1), and (GGGSGGGS)ₙ(SEQ ID NO:2), wherein n is 1-10.

In some embodiments, the immunosuppressive factor is selected from the group consisting of CD47, CD24, CD200, HLA-G, HLA-E, HLA-C, HLA-E heavy chain, PD-L1, IDO1, CTLA4-Ig, C1-Inhibitor, IL-10, IL-35, FASL, Serpinb9, CCl21, and Mfge8. In some embodiments, the immunosuppressive factor gene is CD47.

In some embodiments, the degron element is selected from the group consisting of a ligand inducible degron element, a peptidic degron element, and a peptidic proteolysis targeting chimera (PROTAC) element.

In some embodiments, the ligand inducible degron element is selected from a small molecule-assisted shutoff (SMASH) degron element, Shield-1 responsive degron element, auxin responsive degron element, and a rapamycin responsive degron element. In some embodiments, the ligand inducible degron element is a small molecule-assisted shutoff (SMASH) degron element and the exogenous factor is asunaprevir.

In some embodiments, the construct further comprises a 5′ homology arm and a 3′ homology arm for targeted integration to a safe harbor locus. In some embodiments, the safe harbor locus is selected from the group consisting of an AAVS1 locus, a CLBYL locus, a CXCR4 locus, a Rosa26 locus, and a CCR5 locus.

In some embodiments, the isolated cell is an isolated mammalian cell. In some embodiments, the isolated cell is an isolated human cell.

In some embodiments, the isolated human cell further comprises deletion or reduced expression of MHC class I human leukocyte antigens compared to an unmodified human cell. In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments, the isolated human cell further comprises deletion or reduced expression of B2M. In some embodiments, the isolated human cell further comprises deletion or reduced expression of NLRC5. In some embodiments, the isolated human cell is hypoimmunogenic.

In some embodiments, the isolated human cell is selected from the group consisting of a stem cell, embryonic stem cell, pluripotent stem cell, adult stem cell, and a differentiated cell. In some embodiments, the differentiated cell is selected from the group consisting of a cardiac cell, liver cell, kidney cell, pancreatic cell, neural cell, immune cell, mesenchymal cell, and endothelial cell.

In one aspect, provided herein is a construct comprising from 5′ end to 3′ end: a promoter (e.g., a constitutive promoter); an inducible degron element; an optional sequence encoding a flexible linker; and an immunosuppressive factor gene. In another aspect, provided herein is a construct comprising from 5′ end to 3′ end: a promoter (e.g., a constitutive promoter); an immunosuppressive factor gene; an optional sequence encoding a flexible linker; and an inducible degron element.

In some embodiments, the constitutive promoter is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the flexible linker is selected from the group consisting of (GSG)ₙ(SEQ ID NO:3), (GGGS)ₙ (SEQ ID NO:1), and (GGGSGGGS)ₙ(SEQ ID NO:2), wherein n is 1-10.

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

In some embodiments, the degron element is selected from the group consisting of a ligand inducible degron element, a peptidic degron element, and a peptidic proteolysis targeting chimera (PROTAC) element. In some embodiments, the ligand inducible degron element is selected from a small molecule-assisted shutoff (SMASH) degron element, Shield-1 responsive degron element, auxin responsive degron element, and rapamycin responsive degron element. In certain embodiments, the ligand inducible degron element is a small molecule-assisted shutoff (SMASH) degron element.

In some embodiments, the construct further comprises a 5′ homology arm and a 3′ homology arm for targeted integration to a genomic safe harbor locus. In some embodiments, the genomic safe harbor locus is selected from the group consisting of an AAVS1 locus, a CLBYL locus, a CXCR4 locus, a Rosa26 locus, and a CCR5 locus.

Provided herein is a composition comprising an isolated cell comprising any one of the constructs described. In some embodiments, the isolated cell is selected from the group consisting of a stem cell, embryonic stem cell, pluripotent stem cell, and adult stem cell.

Also provided is a composition comprising isolated differentiated cells prepared by culturing any stem cell described herein under differentiation conditions to produce a differentiated cell. In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

Provided herein is a method of treating a patient in need of cell therapy comprising: (a) administering any one of the compositions described to a patient; and (b) exposing the composition to an exogenous factor to activate the inducible degron element promoter, thereby controlling immunogenicity of the cells of the composition.

In one aspect, provided herein is a method for controlling immunogenicity of a cell comprising: (a) obtaining an isolated cell; (b) introducing into the isolated cell: (i) a first construct comprising from 5′ end to 3′ end: a first promoter (e.g., a constitutive promoter) and an immunosuppressive factor gene; (ii) a second construct comprising from 5′ end to 3′ end: a second promoter (e.g., a constitutive promoter) and a nucleic acid sequence encoding Cas9 or a variant thereof, and (ii) a third construct comprising from 5′ end to 3′ end: an inducible RNA polymerase promoter, a guide RNA (gRNA) sequence targeting the immunosuppressive factor, a third promoter (e.g., a constitutive promoter), and a transactivator element corresponding to the inducible RNA polymerase promoter; and (c) exposing the engineered cell to an exogenous factor to activate the transactivator element, thereby controlling the immunogenicity of the cell. In some embodiments, the method further comprises administering the engineered cell to a subject prior to step (c).

In some embodiments, the inducible RNA polymerase promoter of third construct is U6Tet promoter, the transactivator element is a Tet Repressor element (also referred to as a Tet-On transactivator), and the exogenous factor is tetracycline or a derivative thereof.

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8. In some embodiments, the immunosuppressive factor is CD47.

In some embodiments, the first, second and third constitutive promoters are selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the isolated cell is an isolated mammalian cell. In some embodiments, the isolated cell is an isolated human cell.

In some embodiments, the isolated human cell further comprises deletion or reduced expression of MHC class I human leukocyte antigens compared to an unmodified human cell. In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments; the isolated human cell further comprises deletion or reduced expression of B2M. In some embodiments, the isolated human cell further comprises deletion or reduced expression of NLRC5. In some embodiments, the isolated human cell is hypoimmunogenic.

In some embodiments, the isolated human cell is selected from the group consisting of a stem cell, embryonic stem cell, pluripotent stem cell, and adult stem cell.

In another aspect, provided herein is a composition comprising an isolated cell comprising a DNA targeted nuclease system for controlling immunogenicity of the cell comprising: (a) a first element comprising from 5′ end to 3′ end: a first promoter (e.g., a constitutive promoter) and an immunosuppressive factor gene; (b) a second element comprising from 5′ end to 3′ end: a second promoter (e.g., a constitutive promoter) and a nucleic acid sequence encoding Cas9 or a variant thereof; and (c) a third element comprising from 5′ end to 3′ end: an inducible RNA polymerase promoter, a guide RNA (gRNA) sequence targeting the immunosuppressive factor, a third promoter (e.g., a constitutive promoter), and a transactivator element corresponding to the inducible promoter. In some embodiments, immunogenicity of the cell is controllable upon exposing the cell to an exogenous factor to induce activity of the transactivator element.

In some embodiments, the inducible RNA polymerase promoter of the third element is a U6Tet promoter, the transactivator element is a Tet Repressor element (also referred to as a Tet-On transactivator), and the exogenous factor is tetracycline or a derivative thereof.

In some embodiments, the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8. In some embodiments, the immunosuppressive factor is CD47.

In some embodiments, the first, second and third constitutive promoters are selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the isolated cell of the composition is an isolated mammalian cell. In some embodiments, the isolated cell is an isolated human cell.

In some embodiments, the isolated human cell further comprises deletion or reduced expression of MHC class I human leukocyte antigens compared to an unmodified human cell. In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments, the isolated human cell further comprises deletion or reduced expression of B2M. In some embodiments, the isolated human cell further comprises deletion or reduced expression of NLRC5. In some embodiments, the isolated human cell is hypoimmunogenic.

Provided herein is a composition comprising isolated differentiated cells prepared by culturing any one of the stem cells described herein under differentiation conditions to produce a differentiated cell. In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

Also provided is a method of treating a patient in need of cell therapy comprising: (a) administering any one of the compositions outlined herein; and (b) exposing the composition to an exogenous factor to activate the inducible RNA polymerase promoter, thereby controlling immunogenicity of the cells of the composition.

In one aspect, provided herein is a composition comprising an isolated mammalian cell comprising a modification comprising a recombinant nucleic acid sequence encoding a system for conditional expression of one or more immunosuppressive factors. In some embodiments, the expression of the one or more immunosuppressive factors is controllable by an exogenous factor.

In some embodiments, the system comprises an inducible (or regulatable) protein degradation system to reduce protein levels of the one or more immunosuppressive factors. In some embodiments, the inducible protein degradation system is selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

In some embodiments, the system comprises a RNA regulation system to controllably reduce RNA levels of the one or more immunosuppressive factors. In some embodiments, the RNA regulation system is selected from the group consisting of an inducible shRNA, an inducible siRNA, a CRISPR interference (CRISPRi), and a RNA targeting nuclease system.

In some embodiments, the system comprises a DNA regulation system to reduce expression levels of the one or more immunosuppressive factors. In some embodiments, the DNA regulation system is selected from the group consisting of a tissue-specific promoter expression system, an inducible promoter expression system, a molecule regulated riboswitch system, and an inducible nuclease-based genome editing system. In some embodiments, the inducible promoter expression system comprises a U6Tet promoter and a Tet Repressor element. In some embodiments, the tissue-specific promoter is selected from the group consisting of a cardiac cell-specific promoter, hepatocyte-specific promoter, kidney cell-specific promoter, pancreatic cell-specific promoter, neural cell-specific promoter, immune cell-specific promoter, mesenchymal cell-specific promoter, and endothelial cell-specific promoter. In some embodiments, the molecule regulated riboswitch system comprises a theophylline regulated riboswitch or a guanine regulated riboswitch.

In some embodiments, the inducible nuclease-based genome editing system comprises one selected from the group consisting of CRISPR genome editing comprising an inducible guide RNA targeting the one or more immunosuppressive factors, inducible TALEN genome editing, inducible ZFN genome editing, and small molecule enhanced CRISPR-based genome editing.

In some embodiments, the one or more immunosuppressive factors are selected from the group consisting of CD47, CD24, CD200, HLA-G, HLA-E, HLA-C, HLA-E heavy chain, PD-L1, IDO1, CTLA4-Ig, C1-Inhibitor, IL-10, IL-35, FASL, Serpinb9, CCl21, and Mfge8. In some embodiments, the one or more immunosuppressive factors is CD47.

In some embodiments, the isolated cell of the composition is an isolated mammalian cell. In some embodiments, the isolated cell is an isolated human cell.

In some embodiments, the isolated human cell further comprises deletion or reduced expression of MHC class I human leukocyte antigens compared to an unmodified human cell. In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments, the isolated human cell further comprises deletion or reduced expression of B2M. In some embodiments, the isolated human cell further comprises deletion or reduced expression of NLRC5. In some embodiments, the isolated human cell is hypoimmunogenic. In some embodiments, the isolated mammalian cell or the isolated human cell is selected from the group consisting of a stem cell, embryonic stem cell, pluripotent stem cell, and adult stem cell.

Provided herein is a composition comprising an isolated differentiated cell prepared by culturing any stem cell outlined herein under differentiation conditions to produce a differentiated cell. In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

Also provided is a method of treating a patient in need of cell therapy comprising: (a) administering any composition described herein: and (b) exposing the composition to an exogenous factor to control expression of the one or more immunosuppressive factors, thereby controlling immunogenicity of the cells of the composition.

In one aspect, provided herein is a composition comprising an isolated mammalian cell comprising a recombinant nucleic acid sequence encoding a system for conditional expression of one or more immune signaling factors (e.g., immune signaling proteins). In some embodiments, the expression of the one or more immune signaling factors is controllable by an exogenous factor.

In some embodiments, the system for conditional expression comprises an induced inducible stabilization system to increase protein levels of the one or more immune signaling factors. The inducible protein stabilization system comprises a ligand-inducible protein stabilization system and a small molecule-inducible protein stabilization system.

In some embodiments, the system for conditional expression comprises an RNA regulation system to increase RNA levels of the one or more immune signaling factors. In some embodiments, the RNA regulation system comprises a CRISPR activation (CRISPRa) system.

In some embodiments, the system for conditional expression comprises a DNA regulation system to increase expression levels of the one or more immune signaling factors. In some embodiments, the DNA regulation system comprises one selected from the group consisting of a CRISPR activation (CRISPRa) system, a tissue-specific promoter, an inducible promoter, and a molecule regulated riboswitch system. In some embodiments, the tissue-specific promoter is selected from the group consisting of a cardiac cell-specific promoter, hepatocyte-specific promoter, kidney cell-specific promoter, pancreatic cell-specific promoter, neural cell-specific promoter, immune cell-specific promoter, mesenchymal cell-specific promoter, and endothelial cell-specific promoter. In some embodiments, the inducible promoter comprises a TetOn system. In some embodiments, the molecule regulated riboswitch system comprises a theophylline regulated riboswitch or a guanine regulated riboswitch.

In some embodiments, the one or more immune signaling factors are selected from the group consisting of beta-2-microglobulin (B2M), MHC class I chain-related protein A (MIC-A), MHC class I chain-related protein B (MIC-B), HLA-A, HLA-B, HLA-C, RFXANK, CTLA4, PD1, and ligands of NKG2D. In some embodiments, the one or more immune signaling factors are selected from the group consisting of B2M, MIC-A, MIC-B, HLA-A, HLA-B, HLA-C, RFXANK, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some embodiments, the isolated cell of the composition is an isolated mammalian cell. In some embodiments, the isolated cell is an isolated human cell.

In some embodiments, the isolated human cell further comprises deletion or reduced expression of MHC class I human leukocyte antigens compared to an unmodified human cell.

In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments, the isolated human cell further comprises deletion or reduced expression of B2M. In some embodiments, the isolated human cell further comprises deletion or reduced expression of NLRC5. In some embodiments, the isolated human cell is hypoimmunogenic. In some embodiments, the isolated mammalian cell or the isolated human cell is selected from the group consisting of a stem cell, embryonic stem cell, pluripotent stem cell, and adult stem cell.

Provided herein is a composition comprising isolated differentiated cells prepared by culturing any stem cell described herein under differentiation conditions to produce a differentiated cell. In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

Also provided is a method of treating a patient in need of cell therapy comprising: (a) administering any one of the compositions described herein to a patient; and (b) exposing the composition to an exogenous factor to control expression of the one or more immunosuppressive factors, thereby controlling immunogenicity of the cells of the composition.

In one aspect, provided herein is method for controlling the immunogenicity of a cell, the method comprising: (a) obtaining an isolated cell; (b) introducing into the isolated cell a nucleic acid construct comprising from 5′ end to 3′ end: an inducible RNA polymerase promoter; an immune signaling factor gene; a promoter (e.g., a constitutive promoter); and a transactivator element corresponding to the inducible RNA polymerase promoter to produce an engineered cell; and (c) exposing the engineered cell to an exogenous factor to activate the transactivator element, thereby controlling the immunogenicity of the cell. In some embodiments, the method further comprises administering the engineered cell to a subject prior to step (c).

In another embodiment, the method for controlling the immunogenicity of a cell comprises: (b) introducing into the isolated cell (i) a nucleic acid comprising an inducible RNA polymerase promoter operably linked to an immune signaling factor gene and (ii) a promoter (e.g., a constitutive promoter) operably linked to a transactivator element corresponding to the inducible RNA polymerase promoter to produce an engineered cell.

In yet another embodiment, the method for controlling the immunogenicity of a cell comprises: (b) introducing into the isolated cell a single construct comprising (i) a nucleic acid comprising an inducible RNA polymerase promoter operably linked to an immune signaling factor gene and (ii) a promoter (e.g., a constitutive promoter) operably linked to a transactivator element corresponding to the inducible RNA polymerase promoter to produce an engineered cell.

In some embodiments, the inducible RNA polymerase promoter is a TRE promoter and the transactivator element is a Tet-On element, and the exogenous factor is tetracycline or a derivative thereof.

In some embodiments, the immune signaling factor is selected from the group consisting of B2M, MIC-A/B, HLA-A, HLA-B, HLA-C, RFXANK, CTLA4, PD1, and ligands of NKG2D (e.g., MICA, MICB, RAET1E/ULBP4, RAET1G/ULBP5, RAET1H/ULBP2, RAET1/ULBP1, RAET1L/ULBP6, and RAET1N/ULBP3).

In some embodiments, the constitutive promoter is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the construct comprises from 5′ end to 3′ end: a TRE promoter, an immune signaling factor gene, an EF1a promoter, and a Tet-On element and the exogenous factor is tetracycline or a derivative thereof. In some embodiments, the construct comprises from 5′ end to 3′ end: a TRE promoter, a B2M gene, an EF1a promoter, and a Tet-On element and the exogenous factor is tetracycline or a derivative thereof.

In some embodiments, the construct further comprises a vector backbone for lentiviral expression.

In some embodiments, the isolated cell of the composition is an isolated mammalian cell. In some embodiments, the isolated cell is an isolated human cell.

In some embodiments, the isolated human cell further comprises deletion or reduced expression of MHC class I human leukocyte antigens compared to an unmodified human cell.

In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments, the isolated human cell further comprises deletion or reduced expression of B2M. In some embodiments, the isolated human cell further comprises deletion or reduced expression of NLRC5. In some embodiments, the isolated human cell is hypoimmunogenic.

In some embodiments, the isolated human cell is selected from the group consisting of a stem cell, embryonic stem cell, pluripotent stem cell, adult stem cell, and a differentiated cell. In some embodiments, the differentiated cell is selected from the group consisting of a cardiac cell, liver cell, kidney cell, pancreatic cell, neural cell, immune cell, mesenchymal cell, and endothelial cell.

Also described herein is a construct comprising from 5′ end to 3′ end: an inducible RNA polymerase promoter; an immune signaling factor gene; a constitutive promoter; and a transactivator element corresponding to the inducible RNA polymerase promoter. In some embodiments, the inducible RNA polymerase promoter is a TRE promoter.

In some embodiments, the immune signaling factor is selected from the group consisting of B2M, MIC-A/B, HLA-A, HLA-B, HLA-C, RFXANK, CTLA-4, PD-1, and ligands of NKG2D (e.g., MICA, MICB, RAET1E/ULBP4, RAET1G/ULBP5, RAET1H/ULBP2, RAET1/ULBP1, RAET1L/ULBP6, and RAET1N/ULBP3). In some embodiments, the immune signaling factor is B2M.

In some embodiments, the constitutive promoter of the construct is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

In some embodiments, the construct comprises from 5′ end to 3′ end: a TRE promoter, an shRNA sequence targeting CD47, an EF1a promoter, and a Tet Repressor element.

In some embodiments, any one of the constructs outlined comprises a vector backbone for lentiviral expression.

Provided herein is a composition comprising an isolated cell comprising any one of the constructs described. In some embodiments, the isolated cell is exposed to an exogenous factor to activate the transactivator element. In some embodiments, the isolated cell is selected from the group consisting of a stem cell, embryonic stem cell, pluripotent stem cell, and adult stem cell.

Also, provided is a composition comprising isolated differentiated cells prepared by culturing any stem cell described herein under differentiation conditions to produce a differentiated cell. In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

In another aspect, provided is a method of treating a patient in need of cell therapy comprising: (a) administering any one of the compositions described to a patient; and (b) exposing the composition to an exogenous factor to activate the inducible RNA polymerase promoter, thereby controlling immunogenicity of the cells of the composition.

Also provided herein are engineered iPSCs that controllably overexpress immunosuppressive factors (e.g., hypoimmunity factors and complement inhibitors). Notably, a mechanism to exit hypoimmunity is required as a safety feature against infection or cell transmission between subjects. In one embodiment, regulated expression of an immunosuppressive factor is achieved by inducible expression of an shRNA to knockdown the immunosuppressive factor. In another embodiment, regulated expression is achieved using a small molecule based degron system that targets the immunosuppressive factor for proteasomal degradation

In some embodiments, provided herein is a pluripotent stem cell comprising reduced or silenced expression of MHC class I molecules and/or MHC class II molecules, overexpression of CD47, and an inducible shRNA targeting CD47.

In some embodiments, provided herein is a pluripotent stem cell comprising reduced or silenced expression of B2M and CIITA, overexpression of CD47, and an inducible shRNA targeting CD47.

In some embodiments, provided herein is a pluripotent stem cell comprising reduced or silenced expression of MHC class I molecules and/or MHC class II molecules, overexpression of CD47, and an inducible degron element controlling CD47.

In some embodiments, provided herein is a pluripotent stem cell comprising reduced or silenced expression of B2M and CIITA, overexpression of CD47, and an inducible degron element controlling CD47.

In some embodiments, provided herein is a pluripotent stem cell comprising reduced or silenced expression of MHC class I molecules and/or MHC class II molecules, overexpression of CD47, and a SMASH degron element controlling CD47.

In some embodiments, provided herein is a pluripotent stem cell comprising reduced or silenced expression of B2M and CIITA, overexpression of CD47, and a SMASH degron element controlling CD47.

In some embodiments, provided herein is a pluripotent stem cell comprising reduced or silenced expression of MHC class I molecules and/or MHC class II molecules, overexpression of CD47, a Cas9 or a variant thereof, and an inducible guide RNA targeting CD47.

In some embodiments, provided herein is a pluripotent stem cell comprising reduced or silenced expression of B2M and CIITA, overexpression of CD47, a Cas9 or a variant thereof, and an inducible guide RNA targeting CD47.

In further embodiments, provided herein is a pluripotent stem cell comprising reduced or silenced expression of MHC class I molecules and/or MHC class II molecules, overexpression of CD47, an inducible protein degradation system for modulating expression of CD47, wherein the inducible protein degradation system is selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

In further embodiments, provided herein is a pluripotent stem cell comprising reduced or silenced expression of B2M and CIITA, overexpression of CD47, an inducible protein degradation system for modulating expression of CD47, wherein the inducible protein degradation system is selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

In further embodiments, provided herein is a pluripotent stem cell comprising reduced or silenced expression of MHC class I molecule and/or MHC class II molecule, overexpression of CD47, an RNA regulation system for modulating expression of CD47, wherein the RNA regulation system is selected from the group consisting of an inducible shRNA, an inducible siRNA, a CRISPR interference (CRISPRi), and a RNA targeting nuclease system.

In further embodiments, provided herein is a pluripotent stem cell comprising reduced or silenced expression of B2M and CIITA, overexpression of CD47, an RNA regulation system for modulating expression of CD47, wherein the RNA regulation system is selected from the group consisting of an inducible shRNA, an inducible siRNA, a CRISPR interference (CRISPRi), and a RNA targeting nuclease system.

In further embodiments, provided herein is a pluripotent stem cell comprising reduced or silenced expression of MHC class I molecule and/or MHC class II molecule, overexpression of CD47, a DNA regulation system for modulating expression of CD47, wherein the DNA regulation system is selected from the group consisting of a tissue specific promoter expression system, an inducible promoter expression system, a molecule regulated riboswitch system, and an inducible nuclease-based genome editing system.

In further embodiments, provided herein is a pluripotent stem cell comprising reduced or silenced expression of B2M and CIITA, overexpression of CD47, a DNA regulation system for modulating expression of CD47, wherein the DNA regulation system is selected from the group consisting of a tissue specific promoter expression system, an inducible promoter expression system, a molecule regulated riboswitch system, and an inducible nuclease-based genome editing system.

In some embodiments, provided herein is a pluripotent stem cell comprising reduced or silenced expression of MHC class I molecules and/or MHC class II molecules, overexpression of CD47, and an inducible system for modulating (e.g., decreasing) expression of CD47.

In some embodiments, provided herein is a pluripotent stem cell comprising reduced or silenced expression of B2M and CIITA, overexpression of CD47, and an inducible system for modulating (e.g., decreasing) expression of CD47.

In some embodiments, provided herein is a differentiated cell derived from any one of the pluripotent stem cells outlined herein. In some embodiments, the differentiated cell is selected from the group consisting of a cardiac cell, liver cell, kidney cell, pancreatic cell, neural cell, immune cell, mesenchymal cell, and endothelial cell.

In some embodiments, the present technology provides hypoimmunogenic pluripotent cells that comprise a “safety switch” such as a system for DNA downregulation of an immunosuppressive factor, RNA downregulation of an immunosuppressive factor, protein downregulation of an immunosuppressive factor, DNA upregulation of an immune signaling factor, RNA upregulation of an immune signaling factor, and protein upregulation of an immune signaling factor. These are incorporated to function as a “safety switch” that can cause the death of the hypoimmunogenic pluripotent cells or hypoimmunogenic differentiated cells should they grow and divide in an undesired manner in a recipient subject and an engrafted site. In some embodiments, the present technology also includes utilization of a suicide gene selected from EGFRt, HSVtk, cytosine deaminase, iCaspase9, and NTR.

2. Codependency of Safety Switches and Immunosuppressive Factors

In one aspect, provided herein is a construct comprising from 5′ to 3′ end: (1) a safety switch transgene; (2) a ribosomal skipping sequence and/or a sequence encoding a linker; (3) a hypoimmunity gene. In some embodiments, the construct also comprises a transcriptional regulatory element operably linked to the safety switch transgene and a polyadenylation sequence at the 3′ end of the hypoimmunity gene. In some embodiments, any of the constructs also comprises a vector backbone for lentiviral expression.

In another aspect, provided herein is a construct comprising from 5′ to 3′ end: (1) a hypoimmunity gene; (2) a ribosomal skipping sequence or a linker; (3) a safety switch transgene. In some embodiments, the construct also comprises a transcriptional regulatory element operably linked to the hypoimmunity gene and a polyadenylation sequence at the 3′ end of the safety switch transgene. In some embodiments, any of the constructs also comprises a vector backbone for lentiviral expression.

In some embodiments, the safety switch transgene of the construct is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

In some embodiments, the ribosomal skipping sequence comprises a sequence encoding an IRES sequence or a sequence encoding a 2A-coding sequence.

In some embodiments, the 2A-coding sequence is selected from the group consisting of T2A, P2A, E2A, and F2A.

In some embodiments, the linker is selected from any one of the linkers provided in Table 3.

In some embodiments, the hypoimmunity gene is selected from the group consisting of: CD47, CD24, CD200, HLA-G, HLA-E, HLA-C, HLA-E heavy chain, PD-L1, IDO1 CTLA4-Ig, IL-10, IL-35, FASL, Serpinb9, CCl21, and Mfge8.

In some embodiments of the construct, the transcriptional regulatory element is selected from the group consisting of an eukaryotic translation elongation factor 1 al (EF1A) promoter, an eukaryotic translation elongation factor 1 al short form (EFS) promoter, a cytomegalovirus immediate-early enhancer/promoter (CMV promoter), a CMV early enhancer fused to modified chicken I3-actin (CAGGS) promoter, a Simian virus 40 (SV40) promoter, a copia transposon (COPIA) promoter, an actin 5C (ACT5C) promoter, a tetracycline-responsive promoter element (TRE promoter), a CMV early enhancer fused to modified chicken β-actin (CBh) promoter, a phosphoglycerate kinase 1 (PGK) promoter, and a ubiquitin C (UBC) promoter.

Provided herein are methods of delivering a construct into an isolated cell. The method comprises transducing an isolated cell with a lentiviral construct comprising any of the construct described above; and selecting an engineered cell carrying the safety switch transgene and the hypoimmunity gene.

Also provided is an isolated cell or a population thereof comprising any of the construct described above.

In some embodiments, the construct has been introduced into a target gene locus.

In some embodiments, the target gene locus is selected from the group consisting of a safe harbor locus and an immune signaling gene locus.

In some embodiments, the safe harbor locus is selected from the group consisting of an AAVS1 locus, a CLBYL locus, a CXCR4 locus, a Rosa26 locus, and a CCR5 locus.

In some embodiments, the immune signaling gene locus is selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some embodiments, the isolated cell further comprises deletion or reduced expression of MHC class I human leukocyte antigens compared to an unmodified human cell. In some embodiments, the isolated cell further comprises deletion or reduced expression of MHC class II human leukocyte antigens compared to an unmodified cell.

In some embodiments, the isolated cell further comprises deletion or reduced expression of MHC class I and MHC class II human leukocyte antigens compared to an unmodified human cell. In certain embodiments, the isolated cell further comprises deletion or reduced expression of CIITA. In some embodiments, the isolated cell further comprises deletion or reduced expression of B2M. In particular embodiments, the isolated cell further comprises deletion or reduced expression of NLRC5. In some embodiments, the isolated cell is hypoimmunogenic.

In some embodiments, the isolated cell is selected from the group consisting of a stem cell, embryonic stem cell, pluripotent stem cells and adult stem cell.

Provided herein is a differentiated cell or a population thereof prepared by culturing any one of the stem cells described above under differentiation conditions to produce a differentiated cell or a population thereof.

In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

Additionally, described herein is a method of treating a patient in need of cell therapy comprising administering to patient a differentiated cell or a population thereof as outlined.

Provided herein is a method of treating a patient comprising activating a safety switch in a patient previously administered the differentiated cell or the population thereof as described herein.

In some aspects, provided herein is a construct for homology directed repair into a safe harbor locus comprising from 5′ to 3′ end: (1) a first homology arm homologous to a first endogenous sequence of a safe harbor locus; (2) a safety switch transgene; (3) a ribosomal skipping sequence and/or a sequence encoding a linker; (4) an hypoimmunity gene; (5) a polyadenylation sequence; and (6) a second homology arm homologous to a second endogenous sequence of the safe harbor locus.

In some aspects, provided herein is a construct for homology directed repair into a safe harbor locus comprising from 5′ to 3′ end: (1) a first homology arm homologous to a first endogenous sequence of an immune signaling gene locus; (2) a safety switch transgene; (3) a ribosomal skipping sequence and/or a sequence encoding a linker; (4) an hypoimmunity gene; (5) a polyadenylation sequence; and (6) a second homology arm homologous to a second endogenous sequence of the immune signaling gene locus.

In some embodiments, the construct further comprises a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter located at the 5′ end of the safety switch transgene.

In some embodiments, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

In some embodiments, the ribosomal skipping sequence comprises a sequence encoding an IRES sequence or a sequence encoding a 2A-coding sequence. In certain embodiments, the 2A-coding sequence is selected from the group consisting of T2A, P2A, E2A, and F2A such as those shown in Table 2.

In some embodiments, the linker is selected from any one in Table 3.

In some embodiments, the hypoimmunity gene is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, and Mfge8.

In some embodiments, the safe harbor locus is selected from the group consisting of an AAVS1 locus, a CLBYL locus, a CXCR4 locus, a Rosa26 locus, and a CCR5 locus.

In some embodiments, the immune signaling gene locus is selected from the group consisting of an B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some embodiments, the construct enables a targeting nuclease to cleave the safe harbor locus or the immune signaling gene locus, thereby allowing the construct to recombine into the locus by homology directed repair.

In another aspect, provided herein is an isolated cell or a population thereof comprising a safety switch transgene and a hypoimmunity gene integrated into a safe harbor locus or an immune signaling gene locus, wherein the construct of any one of claims 29-39 has recombined into the endogenous safe harbor locus of a cell, or wherein the construct of any one of claims 30-39 has recombined into the endogenous targeted gene locus of a cell.

In some embodiments, the isolated cell further comprises deletion or reduced expression of MHC class I human leukocyte antigens compared to an unmodified human cell. In some embodiments, the isolated cell further comprises deletion or reduced expression of MHC class II human leukocyte antigens compared to an unmodified cell. In some embodiments, the isolated cell further comprises deletion or reduced expression of MHC class I and MHC class II human leukocyte antigens compared to an unmodified human cell.

In some embodiments, the isolated cell further comprises deletion or reduced expression of CIITA. In some embodiments, the isolated cell further comprises deletion or reduced expression of B2M. In certain embodiments, the isolated cell further comprises deletion or reduced expression of NLRC5. In some embodiments, the isolated cell is hypoimmunogenic.

In some embodiments, the isolated cell is selected from the group consisting of a stem cell, embryonic stem cell, pluripotent stem cells and adult stem cell.

Also provided is a differentiated cell or a population thereof prepared by culturing any one of the stem cells described above under differentiation conditions to produce a differentiated cell or a population thereof.

In some embodiments, the differentiation conditions are appropriate for differentiation of a stem cell into a cell type selected from the group consisting of cardiac cells, liver cell, kidney cells, pancreatic cells, neural cells, immune cells, mesenchymal cells, and endothelial cells.

Also provided is a method of treating a patient in need of cell therapy comprising administering to a patient any differentiated cell or population thereof outlined.

Also provided herein is a method of treating a patient comprising activating a safety switch in a patient previously administered the differentiated cell or the population thereof as described herein.

In some aspects, provided herein is a homology independent donor construct comprising from 5′ to 3′ end: (1) a 5′ long terminal repeats (LTR) comprising a left element (LE); (2) a splice acceptor-viral 2A peptide (SA-2A) element; (3) a safety switch transgene; (4) a ribosomal skipping sequence or sequence encoding a linker; (5) a hypoimmunity gene; (6) a polyadenylation sequence; and (7) 3′ LTR comprising a right element (RE).

In some embodiments, the construct is configured to integrate into a target gene locus of an isolated cell to disrupt expression of the target gene.

In some embodiments, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

In some embodiments, the hypoimmunity factor or gene is selected from the group consisting of: CD47, CD24, CD200, HLA-G, HLA-E, HLA-C, HLA-E heavy chain, PD-L1, IDO1, CTLA4-Ig, IL-10, IL-35, FASL, Serpinb9, CCl21, and Mfge8.

In some embodiments, the target gene locus is immune signaling gene locus selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

In some embodiments, the target gene locus is a safe harbor locus selected from the group consisting of an AAVS1 locus, a CLBYL locus, a CXCR4 locus, a Rosa26 locus, and a CCR5 locus.

Provided herein is an isolated cell or population thereof, wherein any one of the constructs above has integrated into an endogenous target gene to disrupt expression target gene expression in the cell.

In some embodiments, the construct has integrated into the target gene at a nuclease or transposase target site. In some embodiments, both alleles of the target gene are disrupted by nuclease or transposase targeting.

Also provided is a method of delivering a construct into an isolated cell comprising transducing an isolated cell with a lentiviral construct comprising any of the constructs described herein; and selecting an engineered cell carrying the safety switch transgene and the hypoimmunity gene.

In other aspects, any one of the constructs described is integrated into an endogenous target gene to disrupt expression target gene expression in the cell. In some embodiments, the construct is integrated into the target gene at a nuclease or transposase target site.

In yet another aspect, the present disclosure provides a recombinant peptide epitope fusion protein comprising: (1) a hypoimmunity factor; and (2) a surface-exposed peptide epitope heterologous to the hypoimmunity factor.

In some embodiments, the hypoimmunity factor is selected from the group consisting of CD47, CD24, CD200, HLA-G, HLA-E, HLA-C, HLA-E heavy chain, PD-L1, IDO1 CTLA4-Ig, IL-10, IL-35, FASL, Serpinb9, CCl21, Mfge8, and membrane-bound forms thereof.

In some embodiments, the peptide epitope is selected from the group consisting of a CD20 epitope, CCR4 epitope, HER2 epitope, CD19 epitope, MUD epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof, the GD2 epitope is recognized by a therapeutic antibody selected from the group consisting of Hu14.18K322A, Hu14.18-IL2, Hu3F8, dinituximab, c.60C3-RLIc, and biosimilars thereof, the PSMA epitope is recognized by a therapeutic antibody selected from the group consisting of KM2812 and biosimilars thereof; the CD30 epitope or CD16 epitope are recognized by a therapeutic antibody selected from the group consisting of AFM13 and biosimilars thereof, or the CD20 epitope or CD16 epitope are recognized by a therapeutic antibody selected from the group consisting of (CD20)2×CD16 and biosimilars thereof.

In some embodiments, the hypoimmunity factor is at the N-terminus of the fusion protein. In other embodiments, the peptide epitope is at the N-terminus of the fusion protein.

In some embodiments, the fusion protein further comprises a linker connecting the hypoimmunity factor and the peptide epitope. In some embodiments, the fusion protein further comprises another linker located at the N-terminus or C-terminus of the fusion protein. In some embodiments, the linker is selected from any one in Table 2.

In another aspect, provided is a construct encoding a recombinant peptide epitope fusion protein comprising: (1) a sequence encoding a hypoimmunity factor; and (2) a sequence encoding a surface-exposed peptide epitope heterologous to the hypoimmunity factor.

In some embodiments of the construct, the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

In some embodiments, the peptide epitope of the construct is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 epitope or CD16 epitope is recognized by a therapeutic antibody selected from the group consisting of AFM13 and biosimilars thereof, or the CD20 epitope or CD16 epitope are recognized by a therapeutic antibody selected from the group consisting of (CD20)2×CD16 and biosimilars thereof.

In some embodiments, the sequence encoding the hypoimmunity factor is 5′ of the sequence encoding the peptide epitope. In some embodiments, the sequence encoding the peptide epitope is 5′ of the sequence encoding the hypoimmunity factor.

In some embodiments, the construct further comprises a sequence encoding a linker connecting the sequence encoding the hypoimmunity factor and the sequence encoding the peptide epitope. In some embodiments, the construct further comprises a sequence encoding another linker located at the N-terminus or C-terminus of the fusion protein. In some embodiments, the linker is any one in Table 2.

In some embodiments, the construct further comprises the transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter, such as those shown in Table 4.

In some embodiments, the construct further comprises a first homology arm and a second homology arm homologous to a target gene locus for CRISPR-based homology directed repair.

In further embodiments, the construct comprises a vector backbone for lentiviral expression.

Provided herein is a method of delivering a construct into an isolated cell comprising transducing an isolated cell with a lentiviral construct comprising any construct described above; and selecting an engineered cell expressing a hypoimmunity factor-peptide epitope fusion protein.

Additionally, described herein is an isolated cell or a population thereof comprising any construct described above.

In some embodiments, the isolated cell is an isolated human cell.

In some embodiments, the isolated cell further comprises deletion or reduced expression of MHC class I human leukocyte antigens compared to an unmodified human cell.

In some embodiments, the isolated cell further comprises deletion or reduced expression of MHC class II human leukocyte antigens compared to an unmodified cell.

In some embodiments, the isolated cell further comprises deletion or reduced expression of MHC class I and MHC class II human leukocyte antigens compared to an unmodified human cell. In some embodiments, the isolated cell further comprises deletion or reduced expression of CIITA. In some embodiments, the isolated cell further comprises deletion or reduced expression of B2M. In some embodiments, the isolated cell further comprises deletion or reduced expression of NLRC5. In some embodiments, the isolated cell is hypoimmunogenic.

In some embodiments, the isolated cell is selected from the group consisting of a stem cell, embryonic stem cell, pluripotent stem cells and adult stem cell.

Provided herein is a differentiated cell or a population thereof prepared by culturing any stem cell outlined herein under differentiation conditions to produce a differentiated cell or a population thereof.

In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

In some aspects, provided is a method of treating a patient in need of cell therapy comprising administering to patient any differentiated cell or the population thereof as outlined above.

In one aspect, provided is a method of treating a patient comprising administering to a patient previously administered the differentiated cell or the population thereof as outlined above an antibody that binds the peptide epitope. In some embodiments, the antibody mediates ADCC or CDC.

In one aspect, the present disclosure provides a recombinant CD47-internal-peptide epitope fusion protein comprising from N- to C-terminal: (1) a human CD47 fragment comprising a IgV domain of CD47; (2) a first linker; (3) a heterologous peptide epitope (e.g., a heterologous human peptide epitope); (4) a second linker; and (5) a human CD47 transmembrane domain.

In some embodiments of the fusion protein, the human CD47 fragment comprising the IgV domain comprises amino acid residues 1-127 of the human CD47 protein. In some embodiments, the human CD47 transmembrane domain comprises amino acid residues 128-348 of the human CD47 protein.

In some embodiments, the first and second linkers of the fusion protein are selected from any one in Table 2.

In some embodiments, the peptide epitope of the fusion protein is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-IL2, Hu3F8, dinituximab, c.60C3-RLIc, and biosimilars thereof; the PSMA epitope is recognized by a therapeutic antibody selected from the group consisting of KM2812 and biosimilars thereof; the CD30 epitope or CD16 epitope is recognized by a therapeutic antibody selected from the group consisting of AFM13 and biosimilars thereof, or the CD20 epitope or CD16 epitope is recognized by a therapeutic antibody selected from the group consisting of (CD20)2×CD16 and biosimilars thereof.

In another aspect, provided is a construct comprising from 5′ to 3′ end: (1) a transcriptional regulatory element; (2) a sequence encoding a human CD47 fragment comprising a IgV domain; (3) a first linker; (4) a sequence encoding a heterologous peptide epitope (e.g., a heterologous human peptide epitope); (5) a second linker; and (6) a sequence encoding a human CD47 fragment comprising a transmembrane domain and C-terminus.

In some embodiments of the construct, the human CD47 fragment comprising the IgV domain comprises amino acid residues 1-127 of the human CD47 protein. In some embodiments, the human CD47 fragment comprising the transmembrane domain and C-terminus comprises amino acid residues 128-348 of the human CD47 protein. In some embodiments, the first and second linkers are selected from any one in Table 2. In some embodiments, the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof, the CD30 epitope or CD16 epitope is recognized by a therapeutic antibody selected from the group consisting of AFM13 and biosimilars thereof, or the CD20 epitope or CD16 epitope is recognized by a therapeutic antibody selected from the group consisting of (CD20)2×CD16 and biosimilars thereof.

In some embodiments, the transcriptional regulatory element of the construct is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter, such as those shown in Table 4.

In some embodiments, the construct further comprises a first homology arm and a second homology arm homologous to a target gene locus for CRISPR-based homology directed repair.

In certain embodiments, the construct further comprises a vector backbone for lentiviral expression.

In some aspects, provided is a method of delivering a construct into an isolated cell comprising transducing an isolated cell with a lentiviral construct comprising any construct described above; and selecting an engineered cell expressing a CD47-internal-peptide epitope fusion protein.

In some aspects, provided is an isolated cell or a population thereof comprising a construct outlined above.

In some embodiments, the isolated cell is an isolated human cell.

In certain embodiments, the isolated cell further comprises deletion or reduced expression of MHC class I human leukocyte antigens compared to an unmodified human cell. In certain embodiments, the isolated cell further comprises deletion or reduced expression of MHC class II human leukocyte antigens compared to an unmodified cell. In some embodiments, the isolated cell further comprises deletion or reduced expression of MHC class I and MHC class II human leukocyte antigens compared to an unmodified human cell. In some embodiments, the isolated cell further comprises deletion or reduced expression of CIITA. In some embodiments, the isolated cell further comprises deletion or reduced expression of B2M. In other embodiments, the isolated cell further comprises deletion or reduced expression of NLRC5. In some embodiments, the cell is hypoimmunogenic.

In some embodiments, the isolated cell is selected from the group consisting of a stem cell, embryonic stem cell, pluripotent stem cells and adult stem cell.

In some aspects, described herein is a differentiated cell or a population thereof prepared by culturing a stem cell outlined above under differentiation conditions to produce a differentiated cell or a population thereof.

In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

In some aspects, provided is a method of treating a patient in need of cell therapy comprising administering to patient the differentiated cell or the population thereof as described herein.

In one aspect, provided is a method of treating a patient comprising administering to a patient previously administered the differentiated cell or the population thereof as described herein an antibody that binds the peptide epitope. In some embodiments, the antibody mediates ADCC or CDC.

In yet another aspect, provided is a bicistronic construct comprising from 5′ to 3′ end: (1) a transcriptional regulatory element; (2) a sequence encoding a peptide epitope (e.g., a surface-exposed peptide epitope); (3) a ribosomal skipping sequence; and (4) a sequence encoding a hypoimmunity factor.

In some embodiments of the bicistronic construct, the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

In some embodiments, the peptide epitope is selected from the group consisting of a CD20 epitope, CCR4 epitope, HER2 epitope, CD19 epitope, MUD epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 epitope or CD16 epitope is recognized by a therapeutic antibody selected from the group consisting of AFM13 and biosimilars thereof, or the CD20 epitope or CD16 epitope is recognized by a therapeutic antibody selected from the group consisting of (CD20)2×CD16 and biosimilars thereof.

In some embodiments of the bicistronic construct, the ribosomal skipping sequence comprises a sequence encoding an IRES sequence or a sequence encoding a 2A-coding sequence.

In some embodiments, the 2A-coding sequence is selected from the group consisting of T2A, P2A, E2A, and F2A, such as those shown in Table 2.

In some embodiments, the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter, such as those shown in Table 4.

In some embodiments, the bicistronic construct further comprises a first homology arm and a second homology arm homologous to a target gene locus for CRISPR-based homology directed repair. In certain embodiments, the construct further comprises a vector backbone for lentiviral expression.

In some embodiments, provided herein is a method of delivering a construct into an isolated cell comprising transducing an isolated cell with a lentiviral construct comprising a construct outlined above; and selecting an engineered cell expressing the hypoimmunity factor and the peptide epitope.

In some aspects, described is an isolated cell or a population thereof comprising a construct outlined above.

In some embodiments, the isolated cell is an isolated human cell.

In some embodiments, the isolated cell further comprises deletion or reduced expression of MHC class I human leukocyte antigens compared to an unmodified human cell.

In some embodiments, the isolated cell further comprises deletion or reduced expression of MHC class II human leukocyte antigens compared to an unmodified cell. In some embodiments, the isolated cell further comprises deletion or reduced expression of MHC class I and MHC class II human leukocyte antigens compared to an unmodified human cell. In some embodiments, the isolated cell further comprises deletion or reduced expression of CIITA. In some embodiments, the isolated cell further comprises deletion or reduced expression of B2M. In some embodiments, the isolated cell further comprises deletion or reduced expression of NLRC5. In some embodiments, the isolated cell is hypoimmunogenic.

In some embodiments, the isolated cell is selected from the group consisting of a stem cell, embryonic stem cell, pluripotent stem cells and adult stem cell.

In some aspects, described is a differentiated cell or a population thereof prepared by culturing a stem cell as outlined above under differentiation conditions to produce a differentiated cell or a population thereof.

In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

In some aspects, described is a method of treating a patient in need of cell therapy comprising administering to patient the differentiated cell or the population thereof as outlined.

In one aspect, described is a method of treating comprising administering to a patient previously administered the differentiated cell or the population thereof as outlined an antibody that binds to the peptide epitope. In some embodiments, the antibody mediates ADCC or CDC.

Provided herein is a pluripotent stem cell comprising reduced or silenced expression of MHC class I molecules and/or MHC class II molecules, a safety switch transgene and a hypoimmunity gene, wherein expression of the safety switch transgene modulates expression of the hypoimmunity gene.

Provided herein is a pluripotent stem cell comprising reduced or silenced expression of B2M and CIITA, overexpression of CD47, a safety switch transgene and a hypoimmunity gene, wherein expression of the safety switch transgene modulates expression of the hypoimmunity gene.

Provided herein is a pluripotent stem cell comprising reduced or silenced expression of MHC class I molecules and/or MHC class II molecules, a safety switch and a hypoimmunity factor, wherein expression of the safety switch modulates expression of the hypoimmunity factor.

Provided herein is a pluripotent stem cell comprising reduced or silenced expression of B2M and CIITA, overexpression of CD47, a safety switch and a hypoimmunity factor, wherein expression of the safety switch modulates expression of the hypoimmunity factor.

Provided herein is a pluripotent stem cell comprising reduced or silenced expression of MHC class I molecules and/or MHC class II molecules, and a hypoimmunity factor linked to a surface-exposed peptide epitope; wherein the peptide epitope is selected from the group consisting of a CD20 epitope, CCR4 epitope, HER2 epitope, CD19 epitope, MUC1 epitope, EGFR epitope, GD2 epitope, PSMA epitope, CD30 epitope, and CD16 epitope, and the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

Provided herein is a pluripotent stem cell comprising reduced or silenced expression of B2M and CIITA, overexpression of CD47, and comprising a hypoimmunity factor linked to a surface-exposed peptide epitope; wherein the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope, and the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof

3. Codependency of Safety Switches and Essential Cell Factors

In one aspect, provided herein is a construct comprising from 5′ to 3′ end: (1) a safety switch transgene; (2) a ribosomal skipping sequence and/or a sequence encoding a linker; and (3) an essential cell factor gene. In some embodiments, the construct further comprises a transcriptional regulatory element operably linked to the safety switch transgene and a polyadenylation sequence at the 3′ end of the essential cell factor gene. In some embodiments, the construct further comprises a first homology arm and a second homology arm homologous to a target gene locus for CRISPR-based homology directed repair. In other embodiments, the construct comprises a vector backbone for lentiviral expression.

In some aspects, provided herein is a construct comprising from 5′ to 3′ end: (1) an essential cell factor gene; (2) a ribosomal skipping sequence or a linker; and (3) a safety switch transgene. In some embodiments, the construct further comprises a transcriptional regulatory element operably linked to the essential cell factor gene and a polyadenylation sequence at the 3′ end of the safety switch transgene. In some embodiments, the construct further comprises a first homology arm and a second homology arm homologous to a target gene locus for CRISPR-based homology directed repair. In other embodiments, the construct comprises a vector backbone for lentiviral expression.

In one aspect, provided herein is a construct for homology directed repair into a safe harbor locus comprising from 5′ to 3′ end: (1) a first homology arm homologous to a first endogenous sequence of a safe harbor locus; (2) a safety switch transgene; (3) a ribosomal skipping sequence or a sequence encoding a linker; (4) an essential cell factor gene; (5) a polyadenylation sequence; and (6) a second homology arm homologous to a second endogenous sequence of the safe harbor locus.

In another aspect, provided herein is a construct for homology directed repair into a safe harbor locus comprising from 5′ to 3′ end: (1) a first homology arm homologous to a first endogenous sequence of a safe harbor locus; (2) an essential cell factor gene; (3) a ribosomal skipping sequence or a sequence encoding a linker; (4) a safety switch transgene; (5) a polyadenylation sequence; and (6) a second homology arm homologous to a second endogenous sequence of the safe harbor locus

In one aspect, provided herein is construct for homology directed repair into an immune signaling comprising from 5′ to 3′ end: (1) a first homology arm homologous to a first endogenous sequence of an immune signaling gene locus; (2) a safety switch transgene; (3) a ribosomal skipping sequence or a sequence encoding a linker; (4) an essential cell factor gene; (5) a polyadenylation sequence; and (6) a second homology arm homologous to a second endogenous sequence of the immune signaling gene locus.

In one aspect, provided herein is construct for homology directed repair into an immune signaling comprising from 5′ to 3′ end: (1) a first homology arm homologous to a first endogenous sequence of an immune signaling gene locus; (2) an essential cell factor gene; (3) a ribosomal skipping sequence or a sequence encoding a linker; (4) a safety switch transgene; (5) a polyadenylation sequence; and (6) a second homology arm homologous to a second endogenous sequence of the immune signaling gene locus.

In some embodiments, the construct enables a targeting nuclease to cleave the safe harbor locus or the immune signaling gene locus, thereby allowing the construct to recombine into the locus by homology directed repair.

In some aspects, provided herein is a homology independent donor construct comprising from 5′ to 3′ end: (1) a 5′ long terminal repeats (LTR) comprising a left element (LE); (2) a splice acceptor-viral 2A peptide (SA-2A) element; (3) a safety switch transgene; (4) a ribosomal skipping sequence or a sequence encoding a linker; (5) an essential cell factor gene; (6) a polyadenylation sequence; and (7) 3′ LTR comprising a right element (RE).

In one aspect, provided herein is a homology independent donor construct comprising from 5′ to 3′ end: (1) a 5′ long terminal repeats (LTR) comprising a left element (LE); (2) a splice acceptor-viral 2A peptide (SA-2A) element; (3) an essential cell factor gene; (4) a ribosomal skipping sequence or a sequence encoding a linker; (5) a safety switch transgene; (6) a polyadenylation sequence; and (7) 3′ LTR comprising a right element (RE).

In some aspects, provided herein is an isolated cell that is dependent for survival on the expression of the essential gene as part of any of the co-expression constructs described herein. In some embodiments, the isolated cell or a population thereof comprises a recombinant essential cell factor and a safety switch, wherein the endogenous essential cell factor gene has been inactivated (such as excised). In some embodiments, the isolated cell is a homozygous knockout of the essential cell factor gene. In one embodiment, the essential cell factor transgene and the safety switch transgene are introduced into the isolated cell by way of lentiviral delivery. In another embodiment, the essential cell factor transgene and the safety switch transgene are introduced into a safe harbor locus or an immune signaling gene locus.

In some embodiments, any of the cells described are unable to express the essential cell factor from the endogenous loci. In some embodiments, provided herein is a targeting nuclease (such as those described herein) to cleave and inactivate the essential gene locus, thereby rendering the cell dependent for survival on the expression of the essential gene as part of the co-expression construct.

Also provided herein is an isolated cell or a population thereof comprising an essential cell factor gene operably linked to a sequence encoding a linker that is operably linked to a safety switch transgene. In some embodiments, the safety switch is directly linked to the endogenous locus of the essential cell factor gene. In such instances, expression of the essential cell factor is unmodified.

In some embodiments of any of the constructs, cells, and methods, the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD30 transgene, and CD16 transgene.

In other embodiments, the safety switch transgene encodes a sodium/iodide symporter (NIS). NIS-specific radioisotopes (e.g., I-125 and I-131) can be used to target and kill NIS expressing cells.

In some embodiments of the constructs, cells, and methods, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

In some embodiments of the constructs, cells, and methods, the essential cell factor is selected from the group identified as essential genes based on functional genomics screens.

In some embodiments, the safe harbor locus is selected from the group consisting of an AAVS1 locus, a CLBYL locus, a CXCR4 locus, a Rosa26 locus, and a CCR5 locus.

In some embodiments, the immune signaling gene locus is selected from the group consisting of B2M, HLA-A, HLA-B, HLA-C, HLA-D, and HLA-E.

Any of the constructs can be introduced into an isolated cell according to methods recognized by those skilled in the art.

In some embodiments, the isolated cell described herein is an autologous or allogeneic cell. In some embodiments, the isolated cell is an isolated mammalian cell. In some embodiments, the isolated cell is an isolated human cell.

In some embodiments, the isolated human cell further comprises deletion or reduced expression of MHC class I human leukocyte antigens compared to an unmodified human cell. In some embodiments, the isolated human cell further comprises deletion or reduced expression of MHC class II human leukocyte antigens compared to an unmodified human cell. In some embodiments, the isolated human cell further comprises deletion or reduced expression of MHC class I and MHC class II human leukocyte antigens compared to an unmodified human cell. In some embodiments, the isolated human cell further comprises deletion or reduced expression of CIITA. In some embodiments; the isolated human cell further comprises deletion or reduced expression of B2M. In some embodiments, the isolated human cell further comprises deletion or reduced expression of NLRC5. In some embodiments, the isolated human cell is hypoimmunogenic.

In some embodiments, the isolated human cell is selected from the group consisting of a stem cell, embryonic stem cell, pluripotent stem cell, adult stem cell, and a differentiated cell. In some embodiments, the differentiated cell is selected from the group consisting of a cardiac cell, liver cell, kidney cell, pancreatic cell, neural cell, immune cell, mesenchymal cell, and endothelial cell.

Described herein are methods of treating a patient in need of a cell based therapy comprising administering to a patient any of the cells outlined.

Also provided herein is a method of treating a patient comprising activating the safety switch in a patient previously administered the differentiated cell or the population thereof as described herein.

In some aspects, provided herein is a construct comprising (1) a transcriptional regulatory element, (2) an essential cell factor gene, (3) a post-transcriptional or post-translational regulatory element, and (4) a polyadenylation sequence.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

In some embodiments, the transcriptional regulatory element of the construct is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

In some embodiments, the post-transcriptional regulatory element is a RNA regulation system selected from the group consisting of an inducible shRNA, an inducible siRNA, a CRISPR interference (CRISPRi), and a RNA targeting nuclease system.

In some embodiments, the post-translational regulatory element is an inducible protein degradation system is selected from the group consisting of a small molecule-assisted shutoff (SMASH) system, a shield-1-inducible degron, an auxin-inducible degron, an IMid-inducible degron, a peptidic degron, a proteolysis targeting chimera, and an antibody for targeted degradation.

In another aspect, provided herein an isolated cell comprising a recombinant essential cell factor under the control of a post-transcriptional or post-translational regulatory element, wherein the endogenous essential cell factor gene is inactivated and expression of the recombinant essential cell factor is controllable by an exogenous factor.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

In some embodiments, the post-transcriptional regulatory element is a RNA regulation system selected from the group consisting of an inducible shRNA, an inducible siRNA, a CRISPR interference (CRISPRi), and a RNA targeting nuclease system.

In some embodiments, the post-translational regulatory element is an inducible protein degradation system is selected from the group consisting of a small molecule-assisted shutoff (SMASH) system, a shield-1-inducible degron, an auxin-inducible degron, an IMid-inducible degron, a peptidic degron, a proteolysis targeting chimera, and an antibody for targeted degradation.

In some embodiments, the isolated cell is an isolated human cell. In some embodiments, the isolated human cell is an autologous cell or an allogeneic cell.

In some embodiments, the isolated cell further comprises deletion or reduced expression of MHC class I human leukocyte antigens compared to an unmodified human cell. In some embodiments, the isolated cell further comprises deletion or reduced expression of MHC class II human leukocyte antigens compared to an unmodified cell. In some embodiments, the isolated cell further comprises deletion or reduced expression of MHC class I and MHC class II human leukocyte antigens compared to an unmodified human cell. In some embodiments, the isolated cell further comprises deletion or reduced expression of CIITA. In some embodiments, the isolated cell further comprises deletion or reduced expression of B2M. In some embodiments, the isolated cell further comprises deletion or reduced expression of NLRC5. In some embodiments, the isolated cell is hypoimmunogenic.

In some embodiments, the isolated human cell is selected from the group consisting of a stem cell, embryonic stem cell, pluripotent stem cell, adult stem cell, and a differentiated cell. In some embodiments, the differentiated cell is selected from the group consisting of a cardiac cell, liver cell, kidney cell, pancreatic cell, neural cell, immune cell, mesenchymal cell, and endothelial cell.

Described herein are methods of treating a patient in need of a cell based therapy comprising administering to a patient any of the cells outlined.

Also provided herein is a method of treating a patient comprising activating the regulatory in a patient previously administered the differentiated cell or the population thereof as described herein.

In one aspect, provided herein is a recombinant peptide epitope fusion protein comprising: (1) an essential cell factor; and (2) a surface-exposed peptide epitope heterologous to the essential cell factor.

In some embodiments, the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, a spliceosome subunit protein, and membrane-bound forms thereof.

In some embodiments, the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof;

the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In some embodiments, the essential cell factor is at the N-terminus of the fusion protein. In some embodiments, the peptide epitope is at the N-terminus of the fusion protein.

In some embodiments, the fusion protein further comprises a linker connecting the essential cell factor and the peptide epitope. In some embodiments, the fusion protein further comprises another linker located at the N-terminus or C-terminus of the fusion protein.

In some aspects, provided herein is a construct encoding a recombinant peptide epitope fusion protein comprising: (1) a sequence encoding an essential cell factor; and (2) a sequence encoding a surface-exposed peptide epitope heterologous to the essential cell factor.

In some embodiments, the essential cell factor is selected from the group consisting of RpS2. RpS9, RpS11, RpS13, RpS18, RpL8, RpL11, RpL32, RpL36, Rpn22, Psmd14, PSMA3, a ribosome subunit protein, a proteasome subunit protein, a spliceosome subunit protein, and membrane-bound forms thereof.

In some embodiments, the peptide epitope is selected from the group consisting of a CD20 epitope, CCR4 epitope, HER2 epitope, CD19 epitope, MUD epitope, EGFR epitope, GD2 epitope, PSMA epitope, CD16 epitope, and CD30 epitope.

In some embodiments, the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

In some embodiments of the construct, the sequence encoding the essential cell factor is 5′ of the sequence encoding the peptide epitope. In certain embodiments, the sequence encoding the peptide epitope is at the 5′ of the sequence encoding the essential cell factor.

In some embodiments, the construct further comprises a sequence encoding a linker connecting the sequence encoding the essential cell factor and the sequence encoding the peptide epitope.

In some embodiments, the construct further comprises a sequence encoding another linker located at the N-terminus or C-terminus of the fusion protein.

In some embodiments, the construct further comprises a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

In some embodiments, the construct further comprises a first homology arm and a second homology arm homologous to a target gene locus for CRISPR-based homology directed repair.

In certain embodiments, the construct further comprises a vector backbone for lentiviral expression.

Also provided herein is a method of delivering a construct into an isolated cell comprising transducing an isolated cell with a lentiviral construct comprising a construct outlined herein; and selecting an engineered cell expressing a recombinant peptide epitope fusion protein.

Also provided herein is an isolated cell or a population thereof comprising a construct of described above.

In some embodiments, the isolated cell is an isolated human cell. In some embodiments, the isolated human cell is an autologous cell or an allogeneic cell.

In some embodiments, the isolated cell further comprises deletion or reduced expression of MHC class I human leukocyte antigens compared to an unmodified human cell. In some embodiments, the isolated cell further comprises deletion or reduced expression of MHC class II human leukocyte antigens compared to an unmodified cell. In some embodiments, the isolated cell further comprises deletion or reduced expression of MHC class I and MHC class II human leukocyte antigens compared to an unmodified human cell. In some embodiments, the isolated cell further comprises deletion or reduced expression of CIITA. In some embodiments, the isolated cell further comprises deletion or reduced expression of B2M. In some embodiments, the isolated cell further comprises deletion or reduced expression of NLRC5. In some embodiments, the isolated cell is hypoimmunogenic.

In some embodiments, the isolated cell is selected from the group consisting of a stem cell, embryonic stem cell, pluripotent stem cells and adult stem cell.

In one aspect, provided is a differentiated cell or a population thereof prepared by culturing the stem cell described herein under differentiation conditions to produce a differentiated cell or a population thereof. In some embodiments, the differentiation conditions are appropriate for differentiation of a stem cell into a cell type selected from the group consisting of cardiac cells, liver cells, kidney cells, pancreatic cells, neural cells, immune cells, mesenchymal cells, and endothelial cells.

In some aspects, provided is a method of treating a patient in need of cell therapy comprising administering to patient the differentiated cell or the population thereof set forth above.

Also described is a method of treating a patient comprising administering to a patient previously administered the differentiated cell or the population thereof outlined herein an antibody that binds the peptide epitope. In some embodiments, the antibody mediates ADCC or CDC.

### IV. Examples

### A. Example 1: CD47 Downregulation Using Inducible shRNAs Targeting CD47

This example describes the generation and assessment of an inducible shRNA targeting CD47 introduced by lentivirus transduction.

Small hairpin RNAs (shRNA) are sequences of RNA that include a hairpin structure. shRNA molecules are processed within the cell to form siRNA which in turn knock down gene expression. This product is then processed and loaded into the RNA-induced silencing complex (RISC). The processed shRNA directs RISC to mRNA that has a complementary sequence. In the case of perfect complementarity to the CD47 mRNA, RISC cleaves the mRNA, thereby silencing the expression of a gene. An inducible shRNA library was designed to target the mouse and human CD47 mRNA such that, upon induction of shRNA expression by a small molecule, CD47 expression is downregulated.

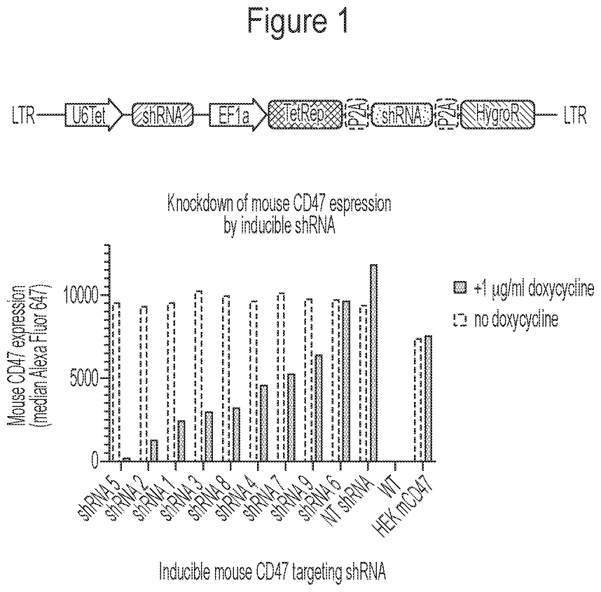

Inducible lentiviral shRNA construct: To generate an inducible lentiviral vector construct, the pRSIT third generation lentiviral plasmid was used as a backbone. A cassette containing shRNA-EF1a-Tet Repressor-2A-TagGFP-2A-Hygromycin was incorporated downstream of the U6Tet promoter, a tet-inducible derivative of the U6 type III RNA polymerase promoter that enables robust shRNA expression (see FIG. 1). The Tet Repressor sequence is a modified Tet-On 3G transactivator which has been evolved to display higher sensitivity to the doxycycline inducer. The Tet Repressor was fused with the T2A peptide onto TagGFP and Hygromycin for selection. We designed 10 such constructs targeting mouse CD47 and 10 targeting human CD47.

Virus production and transduction of cells: For the production of viral particles, HEK293LX cells (Takara) were plated at 5×10⁵ into 10 cm dishes. 24 hours after plating, cells were transfected using polybrene with the following plasmids: 1.5 μg of a VSV-G pseudotyped vector, 3.2 μg of a packaging plasmid containing an empty backbone, an HIV-1 pol, HIV-1 gag, HIV-1 Rev, HIV-1 Tat, an AmpR promoter and an SV40 promoter (psPAX2) and 5.2 μg of the lentiviral transfer vector described above containing a U6Tet-shRNA-EF1a-Tet Repressor-2A-TagGFP-2A-Hygromycin cassette. 48 h after transfection viral supernatants were collected and filtered through a 0.45 μm filter.

Hypo-immune cell line engineering: The target cell for CD47 downregulation is a stem cell (e.g., pluripotent stem cell or induced pluripotent stem cell (iPSC)) that has been modified to express the tolerogenic factor CD47 (mouse or human). Human iPSC undergo two gene-modification steps. In the first step, the human B2M and CIITA genes are knocked out with SpCas9 and guide RNAs targeting the two genes. Detailed descriptions of human B2M and CIITA genetic knockout cells and methods can be found in, for example, WO2016183041 filed May 9, 2015 and WO2018132783 filed Jan. 14, 2018, the disclosures including the sequence listings and Figures are incorporated herein by reference in their entirety. In some instances, overexpression of mouse or human CD47 is achieved by lentiviral transduction, stably integrating an elongation factor 1α short (EFS) promoter and the CD47 gene and puromycin at an MOI of 20.

Transduction of cell lines with shRNA and doxycycline treatment: 5×10⁵ iPSC described above are plated in a 6 well plate and are transduced the next day by spinning with 2 ml media containing virus supernatant and 10 μg/ml protamine sulfate for 30 mn at 800 rpm. The following day, media is changed. 48 hrs later, cells are passaged and are treated with 1 mg/ml doxycycline. Medium containing doxycycline is prepared and changed every second day. Three days later, the transduction efficiency is measured on the basis of the CD47 expression by flow cytometry.

CD47 expression analysis by flow cytometry: After incubation, cells are washed and stained with an anti-CD47 antibody conjugated to Alexa-647 (Biolegend) to detect surface expression of CD47. More specifically, 1×10⁶ cells are harvested and resuspended in 100 μl cell staining-buffer (PBS, 0.1% BSA, 0.1% sodium azide) and incubated with 5 μl Alexa-Fluor 647 labelled anti-CD47 antibody for 30 min on ice. Cells are washed in cell staining buffer and subsequently analyzed by flow cytometry.

FIG. 1 depicts data in a HEK293 cell line engineered to express mouse CD47, showing 98% knockdown efficiency of shRNA #5.

### B. Example 2: CD47 Degradation in Response to the Addition of Small Molecules

This example describes the generation and assessment of a self-cleavable SMASH degron fused to CD47 and inducible Ligand-Induced Degradation (LID) domain fused to CD47 for targeted CD47 degradation.

1. CD47 Degradation Using SMASH Degron

In the SMASH system, the degradation is induced by a small molecule inducer asunaprevir. Described herein is a method for HDR delivery of CD47 modified to include the SMASH tag and a linker into the AAVS1 locus of hypoimmunogenic induced pluripotent (HIP) cells. Also described is a design of the SMASH tag construct and an exemplary method for small molecule treatment to induce degradation of CD47.

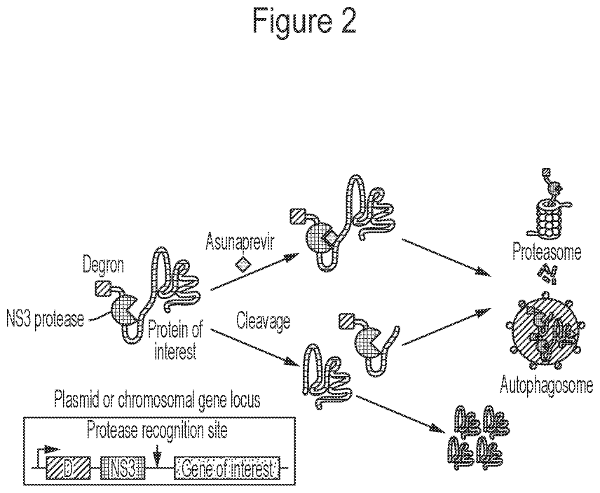

Small-molecule assisted shutoff, or SMASH, is a system using the hepatitis C virus (HCV) nonstructural protein 3 (NS3) protease and elements in the NS4A protein to effectively shut off expression of a CD47 protein fused to a SMASH-tag with clinically tested HCV protease inhibitors (FIG. 2). In the absence of protease inhibitor (asunaprevir), a cryptic degron sequence is excised, leading to an unmodified gene product. By addition of asunaprevir, the NS3 protease is inhibited, leading to the degradation of newly synthesized CD47 proteins fused to the degron sequence.

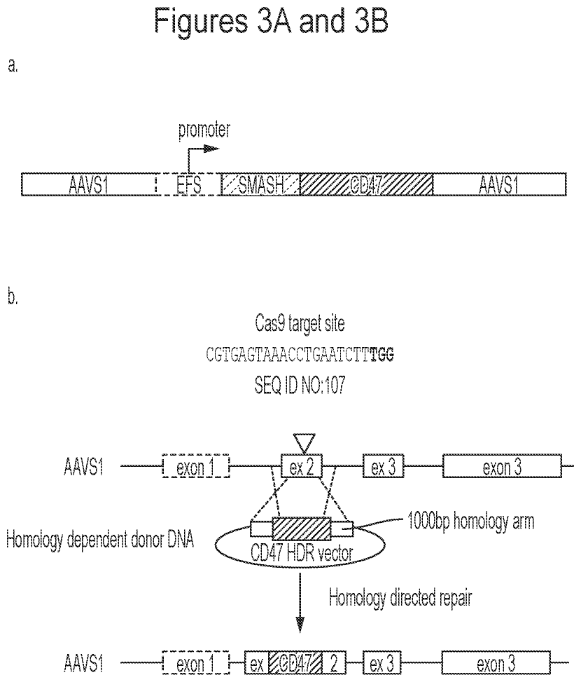

Degron construct: To generate a donor template for homology directed repair (HDR), the pSF plasmid is used as a backbone. A cassette containing an EF1a core promoter (EFS) and a SMASH fused to the human CD47 gene is inserted in between two 1000 bp homology arms to the AAVS1 genomic safe harbor locus (FIG. 3A), hereafter referred to as the AAVS1-EFS-SMASH-CD47-AAVS1 donor template. EFS is a constitutive promoter driving strong expression of the SMASH-CD47 construct. The design of the SMASH tag (see, e.g., Chung et al., Nat. Chem. Bio, 2015; 11:713-720) is such that an NS3 protease-NS4A cassette is fused to a cryptic degron at the N-terminus of CD47 and can remove itself via cleavage of a protease recognition sequence.

Double knockout engineering: The target cell for CD47 downregulation is a stem cell (e.g., pluripotent stem cell or induced pluripotent stem cell) that has been modified to not express MHC molecules class I and II. Human iPSC undergo two gene-modification steps. In the first step, the human B2M and CIITA genes are knocked out with SpCas9 and guide RNAs targeting the two genes. Detailed descriptions of human B2M and CIITA double knockout cells and methods can be found in, for example, WO2016183041 filed May 9, 2015 and WO2018132783 filed Jan. 14, 2018, the disclosures including the sequence listings and Figures are incorporated herein by reference in their entirety. The B2M and CIITA double knockout cell are cultured according to standard methods recognized by those skilled in the art.

Ribonucleoprotein nucleofection of double knockout human iPSC: Following the B2M and CIITA knock out, overexpression of CD47 is achieved by knocking in the CLYBL-CAG-SMASH-CD47-CLYBL cassette into the CLYBL genomic safe harbor locus, or other well-known genomic safe harbor locus such as AAVS1 or CCR5 (FIG. 3B). The CLYBL-CAG-SMASH-CD47-CLYBL plasmid is first linearized by restriction digest with BstBI. A ribonucleoprotein (RNP) mix is prepared as follows: 25 pmol of SpCas9 ribonucleoprotein (RNP) with 75 pmol guide RNA and 1-2 ug linearized donor plasmid into a final volume of 5 μl. Then, 1×10⁶ iPSC are dissociated with accutase and resuspended in 20 μl P3 nucleofection solution (Lonza) and the RNP mix. The cells are nucleofected (Lonza Amaxa nucleofector) using the DN-100 or CA-137 programs and recovered in StemFlex+CloneR and plated on Vitronectin-coated 24 well plate. 10 days later, the bulk edited population is sorted (BD FACS Aria or Hana single cell printer) for high CD47 expression with an anti-CD47 antibody conjugated to Alexa-647, PE or FITC.

Asunaprevir treatment and CD47 downregulation: Sorted cells are treated with about 1-3 μM asunaprevir. Medium containing asunaprevir is prepared and changed about every second day. One day and then three days later, in some embodiments, the transduction efficiency is measured on the basis of CD47 expression by flow cytometry.

CD47 expression analysis: After incubation, cells are washed and stained with an anti-CD47 antibody conjugated to Alexa-647 (Biolegend) to detect surface expression of CD47. More specifically, 1×10⁶ cells are harvested and resuspended in 100 μl cell staining-buffer (PBS, 0.1% BSA, 0.1% sodium azide) and incubated with 5 μl Alexa-Fluor 647 labelled anti-CD47 antibody for 30 min on ice. Cells are washed in cell staining buffer and subsequently analyzed by flow cytometry.

2. Assessment of CD47 Degradation Using SMASH

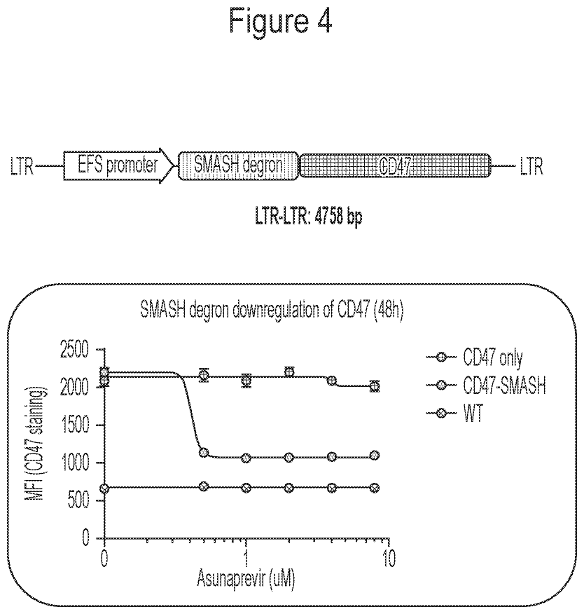

To assess the ability of the SMASH system to promote CD47 degradation, expression constructs were made containing the following expression cassettes: a) an EF1 a core promoter (EFS) operably linked to a human CD47 gene fused to a nucleic acid encoding a SMASH tag (EFS-CD47-SMASH, see FIG. 4, top); and b) a control containing the EFS promoter operably linked to the human CD47 gene alone (EFS-CD47). iPSCs were transduced with either the EFS-SMASH-CD47 expression cassette or control EFS-CD47 expression cassette by lentiviral transduction. The EFS-SMASH-CD47 transduced cells, EFS-CD47 transduced cells, and wild-type control cells were incubated in the presence of 0-10 μM of asunaprevir for 48 hours and CD47 expression was assessed using flow cytometry, as explained above. As shown in FIG. 4 (bottom), the EFS-SMASH-CD47 transduced cells exhibited at least a 50% reduction in CD47 expression with increasing doses of asunaprevir. Expression of CD47 in the EFS-CD47 (“CD47 only”) cells, which did not include SMASH, was generally unaltered by increasing doses of asunaprevir. The EFS-CD47 cells did not show any significant CD47 knockdown, indicating that there were no off-target effects of asunaprevir on CD47 expression. Similarly, expression of CD47 in wildtype cells (“WT”) remained at the baseline (e.g., background) level.

3. CD47 Degradation Using Ligand-Induced Degradation (LID)

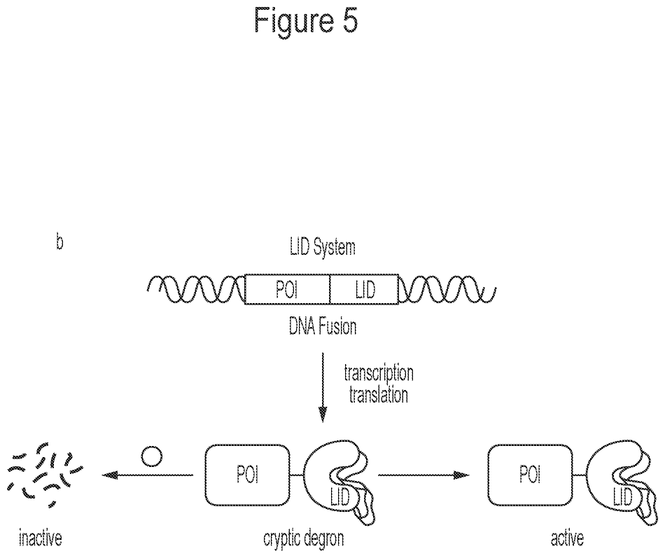

In the LID system, the protein of interest (POI e.g., CD47) is fused to a LID degron domain (also referred to as “LID domain” or “LID degron”). The LID degron domain includes the FK506- and rapamycin-binding protein (FKBP) and a peptide degron (e.g., TRGVEEVAEGVVLLRRRGN) fused to the C-terminus of the FKBP (FIG. 5). FKBP is an enzyme possessing cis/trans prolyl isomerase activity and can active on a broad spectrum of substrate polypeptides. The peptide degron is capable of binding to the FKBP active site and is not detected by cellular degradation proteins when sequestered in the active site, thus rendering it a cryptic degron. In the absence of Shield-1, a small molecule, the POI-LID fusion protein is stable. When present, Shield-1 binds tightly to FKBP, thereby displacing the peptide degron and inducing rapid degradation of the LID and any fused partner protein (e.g., CD47).

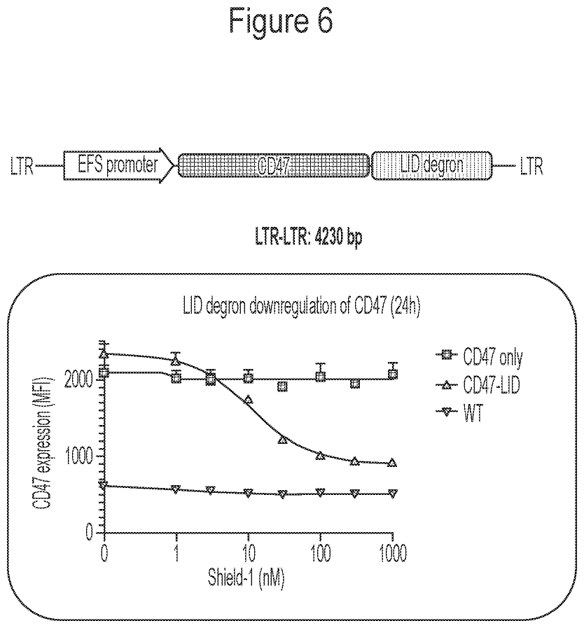

To assess the ability of the LID system to promote CD47 degradation, expression constructs were made containing the following expression cassettes: a) an EF1a core promoter (EFS) operably linked to a human CD47 gene fused to a nucleic acid encoding the LID degron domain (EFS-CD47-LID, see FIG. 6, top); and b) a control containing the EFS promoter operable linked to the human CD47 gene alone (EFS-CD47). iPSCs were transduced with either the EFS-CD47-LID expression cassette or control EFS-CD47 expression cassette by lentiviral transduction.

The EFS-CD47-LID transduced cells, EFS-CD47 transduced cells, and wild-type control cells were incubated in the presence of 0-1,000 nM of Shield-1 for 24 hours and CD47 expression was assessed using flow cytometry. As shown in FIG. 6 (bottom), the EFS-CD47-LID transduced cells exhibited at least a 50% reduction in CD47 expression with increasing doses of Shield-1. Expression of CD47 in the EFS-CD47 (“CD47 only”) cells, which did not include LID, was generally unaltered by increasing doses of Smash-1. The EFS-CD47 cells did not show any significant CD47 knockdown, indicating that there were no off-target effects of Shield-1 on CD47 expression. Similarly, expression of CD47 in wildtype cells (“WT”) remained at the baseline (e.g., background) level.

### C. Example 3: Uncloaking Hypo-Immune Cells Through Genetic, Post-Transcriptional, and Post-Translational Regulation

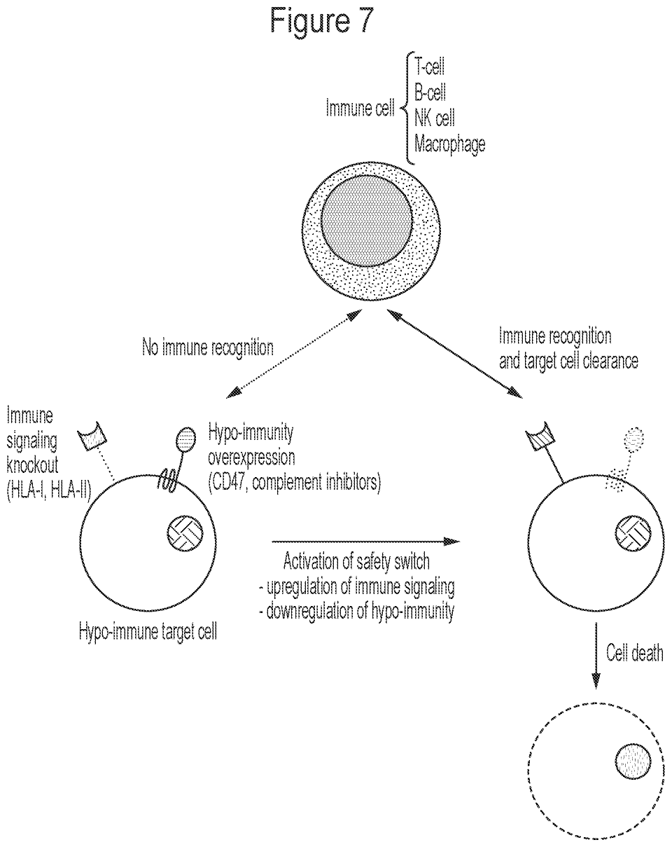

Hypoimmunity is achieved through the overexpression of hypoimmune molecules such as CD47, complement inhibitors accompanied with the repression or genetic disruption of the HLA-I and HLA-II loci. These modifications cloak the cell from the immune system's effector cells that are responsible for the clearance of infected, malignant or non-self cells, such as T-cells, B-cells, NK cells and macrophages. Cloaking of a cell from the immune system allows for existence and persistence of allogeneic cells within the body. Removal of the engineered cells from the body is crucial for patient safety and can be achieved by uncloaking the cells from the immune system. Uncloaking serves as a safety switch and can be achieved through the downregulation of the hypoimmune molecules or the upregulation of immune signaling molecules (FIG. 7). Either of these activities will avail the cell to native effector cells, resulting in clearance of the allogeneic cell.

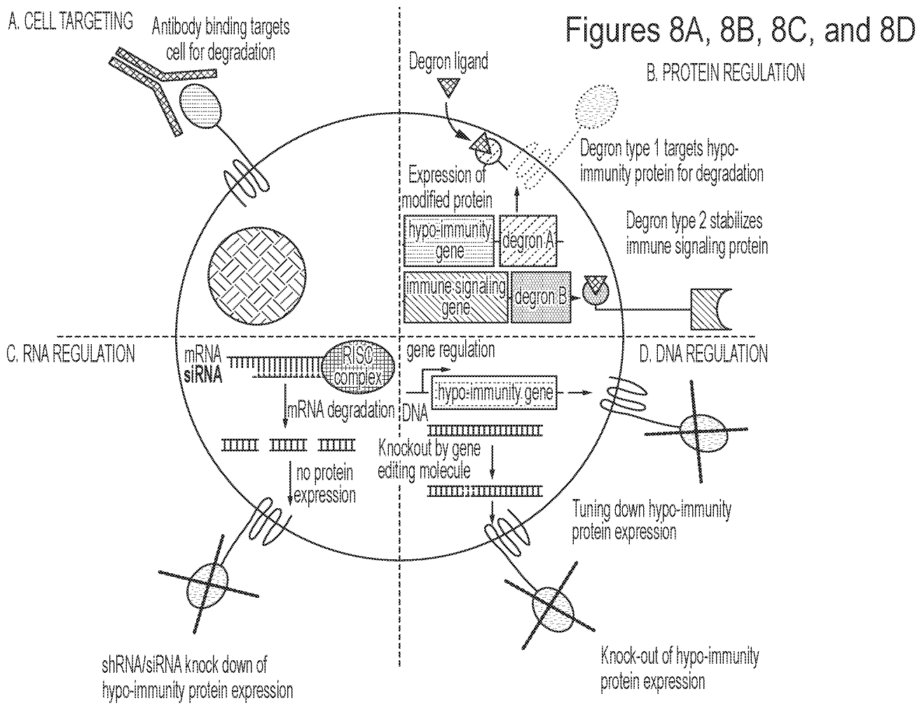

FIG. 8A-FIG. 8D illustrate methods for uncloaking hypo-immune cells through genetic, post-transcriptional, and post-translational regulation. In some embodiments, hypo-immune cells can be availed and cleared by the immune system through the addition of an antibody that binds an epitope on the extracellular surface of the cell (FIG. 8A). The epitope can be native to the overexpressed hypoimmune molecule, or can be another epitope located within the hypoimmune molecule or distinctly located at the extracellular surface. Binding of an antibody to the surface uncloaks the cell and leads to antibody-dependent cellular cytotoxicity (ADCC) or complement-dependent cytotoxicity (CDC). Fusion of inducible degron motifs to hypoimmune molecules enables exogenous control over the stability of the molecule through the addition or removal of small molecules that stabilize or destabilize the degron, and thus the hypoimmune molecule (FIG. 8B). Targeting hypoimmune molecules with siRNAs or miRNAs leads to the degradation of the transcription encoding the protein. An siRNA can be exogenously provided or genetically encoded to provide control over transcription of the inhibitory RNA. The siRNA or miRNA anneals to the hypoimmune molecule's transcript resulting in degradation by the RISC complex (FIG. 8C). Transcriptional regulation of hypoimmune molecules through employing inducible promoters provides the ability to turn expression of the switch on or off at will through the addition or removal of small molecules, such as doxycycline (FIG. 8D). Genetic disruption via targeted nuclease activity will eliminate expression of the hypoimmune molecule to uncloak the cells as well.

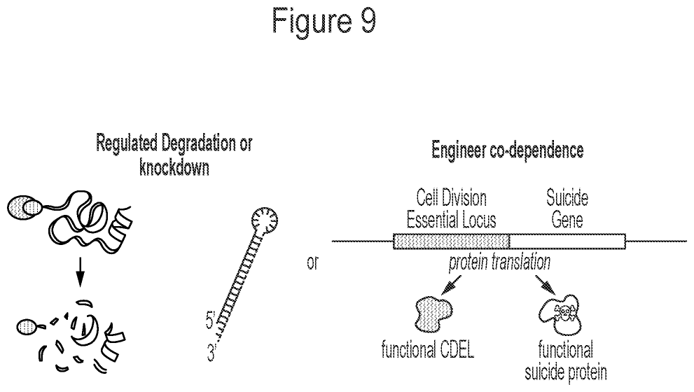

Coupling the expression of a safety switch with a hypoimmune molecule provides a failsafe to ensure expression of the safety switch. The silencing of the cassette encoding the hypoimmune molecule will result in elimination of the cell by the immune system. Furthermore, the cassette containing the hypoimmune molecule and safety switch can be integrated into an endogenous essential locus to safeguard expression of the cassette, as silencing of the essential gene will eliminate the engineered cells (FIG. 9). In some cases, inducible CD47 downregulation in hypoimmune cell acts as a safety switch, thereby allowing the hypoimmune cell to be removed by the immune system.

To illustrate targeted degradation of an hypoimmune molecule using an inducible shRNAs, HEK293 cells overexpressing mouse CD47 were transduced with 9 shRNA candidates, respectively resulting into the integration of heterologous DNAs encoding a doxycycline inducible U6 promoter controlling the expression of the shRNA. In the study, HEK293 WT served as a negative control for mouse CD47 expression. The inducible shRNA was provided to the cells via lentiviral transduction. The cells were transduction at MOI 1.8 with 9 different shRNA constructs targeting the mouse CD47 transcript 1. Also, a non-targeting control was used. Addition of doxycycline at 1 μg/ml for 72 hours conferred downregulation of the CD47 protein as measured by flow cytometry analysis following incubation of cells with an anti-CD47 antibody conjugated to AlexaFluor 647). Data illustrate that 8 of the 9 inducible shRNAs knocked down expression of mouse CD47. Test article shRNA #5 provided complete knockdown of CD47 expression and test articles shRNAs #2, 3, 4, 7, 8, and 9 provided partial knockdown (see FIG. 1).

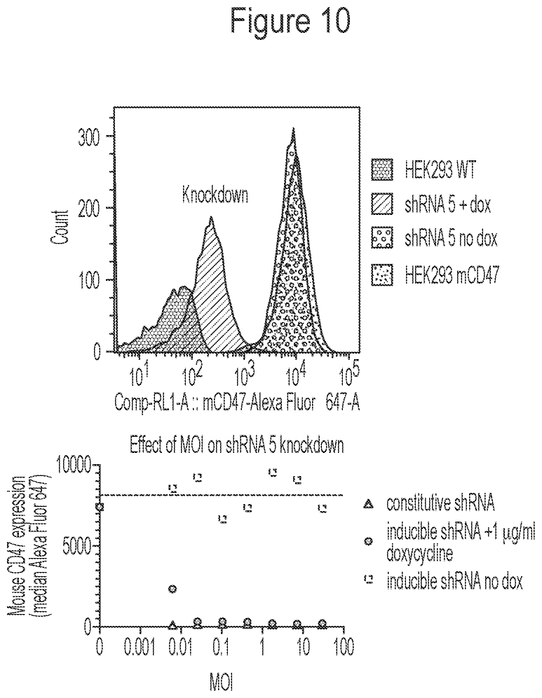

Virus harboring the most effective shRNA (#5 of FIG. 10) was used for transductions at varying MOIs, followed by induction of shRNA expression. Subsequently, analysis of CD47 levels via flow cytometry was performed as described in FIG. 10. Data illustrate that MOI above 0.3 enabled efficient knockdown of CD47 (FIG. 10, bottom). It was noted that inducible shRNA construct was as efficient at CD47 downregulation as a constitutively expressed version of the shRNA.

This example describes methods for generating hypoimmune cells with controllable or inducible expression of a hypoimmune factor. Such cells possess a safety switch that allows for controlled downregulation or degradation of the hypoimmune factor, and thus the cells can be removed by a subject's immune system.

### D. Example 4: CD47 Coexpression with a Safety Switch

This example describes the linking of an epitope to CD47 that allows for the induction of antibody dependent and complement dependent cytotoxicity by immune cells or immune system components.

The expression of CD47 contributes to cloaking allogeneic cells from the immune system. Non-cell based immune system components that are capable of lysing a target cell are complement mediated processes that recognize cells with antibodies bound to their surface. The expression of an epitope at the extracellular surface of the plasma membrane avails the epitope for antibody binding and can be utilized in the context of fusing the epitope to other cell-surface proteins such as CD47. Monoclonal antibodies that have known epitopes, such as the CPYSNPSLCS fragment of CD20 which binds Rituximab, can be fused to the N-terminus of CD47 or within the IgV domain located between residues 19-127 or directly after residue 127. The epitope can be flanked by flexible linkers, such as GS linkers (e.g., GGGS or GGGSGGGS), to maintain structural integrity of CD47. Placement of the epitope directly after residue 127 within CD47 generates a fusion protein whereby the epitope is adjacent to the plasma membrane and between the globular IgV domain.

In another embodiment, CD47 and the epitope are expressed as a bicistronic transcript separated by a 2A or internal ribosomal entry sequence (IRES). In the bicistronic construct, CD47 is a stand alone molecule as well as the epitope, which is fused to a transmembrane domain and signal sequence that localizes the epitope to the extracellular surface of the plasma membrane while being anchored into the membrane through the transmembrane domain.

The CD47-epitope cassette is integrated into the genome of a cell through lentiviral transduction or CRISPR-mediated homology directed repair at a locus of interest. Isolation of cells harboring the integration is performed through incubating cells with an antibody against the epitope or CD47 followed by flow assisted cytometric sorting (FACS). Elimination of the cells via kill switch activity occurs through addition of the monoclonal antibody that binds the epitope, such as Rituximab binding to CD20, followed by ADCC or CDC mediated cytolysis.

CD47-CD20 epitope fusion and bicistronic construct design: DNA is synthesized by a contract research organization to create constructs harboring several components: the EFS promoter, a CD20 epitope comprising the amino acid sequence CPYSNPLSLCS flanked by a 5′ GGGS linker and a 3′ GGGSGGGS linker (hereafter referred to as N terminal CD20 mimotope), human CD47, and a P2A for the bicistronic cassette.

Fusion constructs: The fusion protein cassette is cloned into the pSF lentiviral backbone and contains the EFS promoter, followed by the CD20 mimotope ORF directly fused to the human CD47 ORF. Another fusion protein cassette contains the EFS promoter followed by human CD47 amino acids 1-127, followed by the CD20 mimotope, followed by the downstream CD47 ORF, which is cloned in the pSF backbone for lentiviral mediated genomic integration. In other words, the fusion cassette cloned in the pSF backbone comprises from 5′ to 3′ end: an EFS promoter, human CD47 amino acids 1-127, a CD20 mimotope described herein, and a CD47 ORF.

Bicistronic constructs: For bicistronic cassettes containing CD47 and CD20 mimotope, two separate peptides are generated by using a 2A or IRES sequence. The CD20 mimotope is fused to a transmembrane domain sequence and a signal sequence enabling its localization at the plasma membrane extracellular surface. CD47 is a human codon optimized CD47. All cassettes are cloned into the pSF backbone for lentiviral mediated integration.

Homology directed repair domain DNA constructs: For CRISPR-mediated homology directed repair (HDR), upstream and downstream of the cassette are flanked by 1000 base pair homology arms that are complementary to the region flanking the sgRNA targeting sequence for the Cas effector nuclease. For targeting of B2M, 1000 bp homology arms flanking the guide RNA targeting exon 2.

Methods for genome integration: Lentiviral packaging of the constructs: HEK293LX cells are transfected with pSF-based plasmids harboring the safety switch-CD47 combinations as well as packaging plasmids to package lentivirus. Briefly, 160,000 HEK293LX cells are seeded per well in 24 well plates in 0.5 ml of DMEM (ThermoFisher #1056044) containing 10% FBS (Gibco #26140079). 22 hours after seeding, transfections containing 100 ng VSVG envelope plasmid, 150 ng packaging plasmid, and 250 ng transfer plasmid are mixed in 50 ul of Optimem (ThermoFisher #11058021) followed by addition of 1.5 ul of TransIT (Mirus Bio #MIR2300). The transfection mixes are incubated for 15 minutes and then added dropwise to each well. 24 hours after transfection the media is changed. 48 hours after transfection, the media is transferred to a centrifuge compatible tube and centrifuged at 300 rcf for 4 minutes to pellet cell debris. After centrifugation, the crude lentiviral supernatant is transferred to a new tube which is then concentrated by ultracentrifugation using a sucrose gradient, followed by resuspension of the lentiviral pellet in PBS.

For transduction and isolation of cells harboring genomic integrations, 125,000 iPS cells are seeded onto vitronectin coated plated 24 hours before transduction. Directly before transduction, media is aspirated and 450 ul of media is added followed by 50 ul of concentrated lentivirus. Cells are incubated with lentivirus for 36 hours before media change. 72 hours after transduction, cells are dissociated and subject to incubation with an anti-CD47 antibody conjugated to Alexa-647 followed by FACS sorting for Alexa-647 cells.

To integrate the CD47-safety switch cassettes into B2M via CRISPR-mediated homology directed repair, the plasmid harboring B2M homology arms is linearized by restriction digest with BstBI. Next, a ribonucleoprotein (RNP) mix is prepared as follows: 25 pmol of SpCas9 ribonucleoprotein (RNP) with 75 pmol guide RNA and 1-2 ug linearized donor plasmid into a final volume of 5 ul. Then, 1×10⁶ iPSC are dissociated with accutase and resuspended in 20 ul P3 nucleofection solution (Lonza) and the RNP mix. The cells are nucleofected (Lonza Amaxa nucleofector) using the DN-100 or CA-137 programs and recovered in StemFlex+CloneR and plated on Vitronectin-coated 24 well plate. 10 days later, the bulk edited population is sorted (BD FACS Aria or Hana single cell printer) for high CD47 expression with an anti-CD47 antibody conjugated to Alexa-647, PE or FITC.

Treatment of CD20-CD47 cells with Rituximab and complement to induce cytolysis: To induce complement dependent cell-death on CD20-CD47 harboring cells, 200,000 cells are seeded into a well of a 12 well plate followed by addition of 50 ug/ml of Rituximab (RnD systems #MAB9575). The cells are incubated with the antibody at 37° C. for 1 hour, followed by addition of human serum (Millipore Sigma #H4522), diluted 1:4 in PBS, which is added at a 1:1 ratio to the target cell cultures to produce a final volume of 100 ul. Every 15 minutes for 120 minutes samples are harvested for flow cytometric analysis of cell death via dissociation, pelleting, and resuspension in the Draq7 viability dye (AbCam #ab109202).

### E. Example 5: CD47 Coexpression with a Safety Switch—CD47-HSVtk Switch

This example describes the CD47-HSVtk bicistronic cassette which links CD47 to a safety switch that functions independent of the immune system.

CD47 can be linked to a safety switch that induces cell death in an immune system independent manner, such as HSVtk, iCaspase9, or Cytosine Deaminase. These safety switches can be located upstream or downstream of CD47 located between a 2A sequence or IRES to ensure the two proteins are separate following translation. HSVtk mediates cell death through catalyzing ganciclovir into a toxic nucleoside which is incorporated during DNA replication, by which accumulation renders toxic DNA damage.

HSVtk-CD47 Bicistronic Construct Design: HSVtk and human CD47 ORFs is separated by a 2A or IRES sequence to create a bicistronic gene construct. Specifically, the EFS promoter is placed upstream of HSVtk, followed by a P2A, followed by CD47, followed by a poly adenylation sequence to create the construct. As such, the bicistronic construct comprises from the 5′ to 3′ end: a EFS promoter, HSVtk, a 2A or IRES sequence, human CD47 or a fragment or variant thereof, and a poly adenylation sequence. Cassettes lacking a poly adenylation sequence are packaged into a pSF backbone for lentiviral mediated integration.

To enable homology directed repair mediated by Cas9, cassettes containing a poly adenylation sequence are flanked by homology arms for the target gene. For targeting of B2M, 1000 bp homology arms flanking the guide RNA targeting exon 2 of B2M gene are employed.

Methods for Genome Integration: Lentiviral packaging of the constructs: HEK293LX cells are transfected with pSF-based plasmids harboring the safety switch-CD47 combinations as well as packaging plasmids to package lentivirus. Briefly, 160,000 HEK293LX cells are seeded per well in 24 well plates in 0.5 ml of DMEM (ThermoFisher #1056044) containing 10% FBS (Gibco #26140079). 22 hours after seeding, transfections containing 100 ng VSVG envelope plasmid, 150 ng packaging plasmid, and 250 ng transfer plasmid are mixed in 50 ul of Optimem (ThermoFisher #11058021) followed by addition of 1.5 ul of TransIT (Mirus Bio #MIR2300). The transfection mixes are incubated for 15 minutes and then added dropwise to each well. 24 hours after transfection the media is changed. 48 hours after transfection, the media is transferred to a centrifuge compatible tube and centrifuged at 300 rcf for 4 minutes to pellet cell debris. After centrifugation, the crude lentiviral supernatant is transferred to a new tube which is then concentrated by ultracentrifugation using a sucrose gradient, followed by resuspension of the lentiviral pellet in PBS.

For transduction and isolation of cells harboring genomic integrations, 125,000 iPS cells are seeded onto vitronectin coated plated 24 hours before transduction. Directly before transduction, media is aspirated and 450 ul of media is added followed by 50 ul of concentrated lentivirus. Cells are incubated with lentivirus for 36 hours before media change. 72 hours after transduction, cells are dissociated and subject to incubation with an anti-CD47 antibody conjugated to Alexa-647 followed by FACS sorting for Alexa-647 cells.

To integrate the CD47-safety switch cassettes into B2M via CRISPR-mediated homology directed repair, the plasmid harboring B2M homology arms is linearized by restriction digest with BstBI. Next, a ribonucleoprotein (RNP) mix is prepared as follows: 25 pmol of SpCas9 ribonucleoprotein (RNP) with 75 pmol guide RNA and 1-2 ug linearized donor plasmid into a final volume of 5 ul. Then, 1×10⁶ iPSC are dissociated with accutase and resuspended in 20 ul P3 nucleofection solution (Lonza) and the RNP mix. The cells are nucleofected (Lonza Amaxa nucleofector) using the DN-100 or CA-137 programs and recovered in StemFlex+CloneR and plated on Vitronectin-coated 24 well plate. 10 days later, the bulk edited population is sorted (BD FACS Aria or Hana single cell printer) for high CD47 expression with an anti-CD47 antibody conjugated to Alexa-647, FITC or PE.

Treatment of HSVtk-CD47 cells with ganciclovir to induce cell death: To induce cell death of HSVtk-CD47 containing cells, ganciclovir (G2536) is added at 1 uM to cell cultures. Cell death is analyzed every 24 hours through for flow cytometric analysis of cell death via dissociation, pelleting, and resuspension in the Draq7 viability dye (AbCam #ab109202).

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

### F. Example 6: CD47 Coexpression with a Safety Switch—Cytosine Deaminase

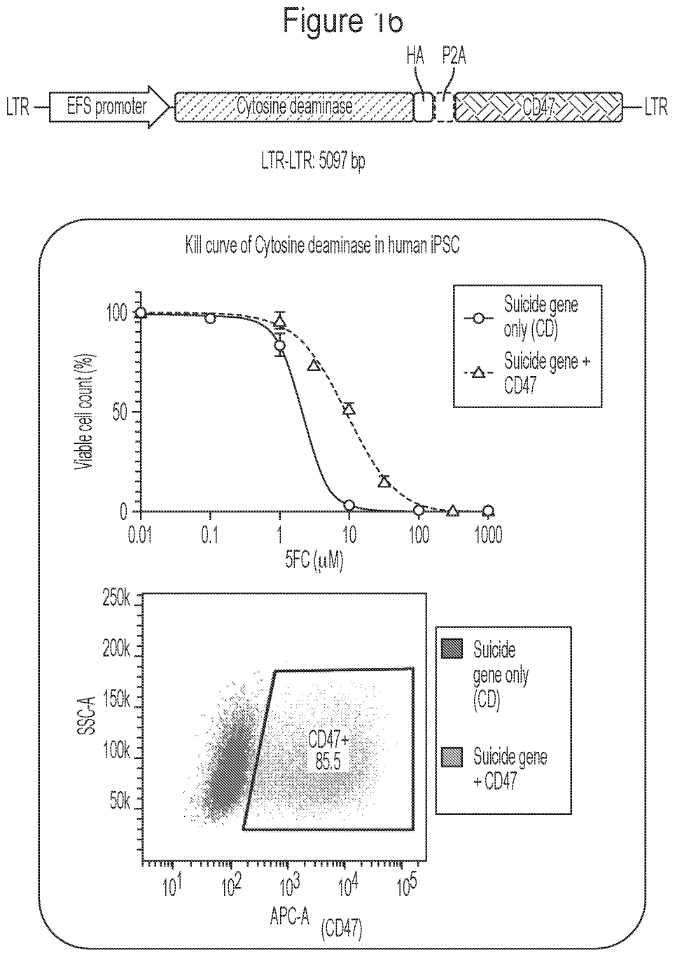

To assess the ability of cytosine deaminase to induce cell death in an immune system dependent manner, an EFS-cytosine deaminase (CD)-CD47 bicistronic cassette was transduced into wild type iPSCs by lentiviral viral transduction (see FIG. 16, top). In this cassette, a nucleic acid encoding CD is located upstream of a nucleic acid encoding CD47. A 2A sequence located between the CD and CD47 nucleic acids to ensure the two proteins are separate following translation. An EFS promoter controls expression of CD and CD47.

EFS-CD-CD47 transduced cells and control EFS-CD transduced cells were contacted with 0.01-1000 μM concentrations of 5-fluorocytosine (5-FC) (“5FC”). Cytosine deaminase deaminates 5-FC to toxic 5-fluorouracil (5-FU), thereby killing the cells. As shown in FIG. 16 (middle), killing of CD47 expressing EFS-CD-CD47 transduced cells was observed with increasing concentrations of 5-FC. Flow cytometry analysis showed that the EFS-CD-CD47 transduced cells expressed CD47 (FIG. 16, bottom). Such co-expression of the CD with CD47 did not significantly affect the functionality of CD when compared with control cells.

TABLE 2
Self-Cleaving Peptide Sequences

| | | SEQ ID |
|---|---|---|
| Self-Cleaving Peptide Name | Sequence | NO |
| T2A self-cleaving peptide | (GSG)EGRGSLLTCGDVEENPGP | 4 |
| sequence | | |
| P2A self-cleaving peptide | (GSG)ATNFSLLKQAGDVEENPGP | 5 |
| sequence | | |
| E2A self-cleaving peptide | (GSG)QCTNYALLKLAGDVESNPGP | 6 |
| sequence | | |
| F2A self-cleaving peptide | (GSG)VKQTLNFDLLKLAGDVESNPGP | 7 |
| sequence | | |

TABLE 3
Useful Linkers

| Linker | | SEQ |
|---|---|---|
| Name | Linker Sequence | ID NO |
| RH_1 | EAAAK | 8 |
| RH_2 | EAAAKEAAAK | 9 |
| RH_3 | EAAAKEAAAKEAAAK | 10 |
| RH_5 | AEAAAKEAAAKEAAAKA | 11 |
| RH_6 | AEAAAKAEAAAKAEAAAKAEAAAK | 12 |
| RH_7 | AEAAAKAEAAAKAEAAAKAEAAAKAEAAAK | 13 |
| RH_8 | AEAAAKEAAAKEAAAKEAAAKALEAEAAAKEAAAKEAAAKEAA | 14 |
| | AKA | |
| REPR_ | PAPAP | 15 |
| 15 | | |
| REPR_ | APAPAPAPAP | 16 |
| 2 | | |
| REPR_ | APAPAPAPAPAPAPAP | 17 |
| 5 | | |
| REPR_ | APAPAPAPAPAPAPAPAPAPAP | 18 |
| 8 | | |
| REPR_ | APAPAPAPAPAPAPAPAPAPAPAPAPAP | 19 |
| 11 | | |
| REPR_ | APAPAPAPAPAPAPAPAPAPAPAPAPAPAPAPAP | 20 |
| 14 | | |
| FGSR_ | KESGSVSSEQLAQFRSLD | 21 |
| 15 | | |
| FGSR_ | EGKSSGSGSESKST | 22 |
| 16 | | |
| FGSR_ | NSGAGGSGGSSGSDGASGSRD | 23 |
| 18 | | |
| FGSR_ | GGGGS | 24 |
| 1 | | |
| FGSR_ | GGGS | 25 |
| 10 | | |
| FGSR_ | GS | 26 |
| 14 | | |
| FGSR_ | GSAGSAAGSGEF | 27 |
| 17 | | |
| FGSR_ | GGGGSGGGGS | 28 |
| 2 | | |
| FGSR_ | GGGGSGGGGSGGGGS | 29 |
| 3 | | |
| FGSR_ | GGGGSGGGGSGGGGSGGGGS | 30 |
| 4 | | |
| FGSR_ | GGS | 31 |
| 5 | | |
| FGSR_ | GGGGGG | 32 |
| 6 | | |
| FGSR_ | GGGGGGGG | 33 |
| 7 | | |
| AP_5 | SSSSG | 34 |
| AP_6 | SSSSGSSSSG | 35 |
| AP_7 | SSSSGSSSSGSSSSG | 36 |
| AP_8 | SSSSGSSSSGSSSSGSSSSG | 37 |
| AP_9 | SSSSGSSSSGSSSSGSSSSGSSSSG | 38 |
| AP_18 | ALSNSIMYFSHFVPVFLPAKPTTTPAPRPPTPAPTIASQPLSLRPEASR | 39 |
| | PAAGGAVHTRGLD | |
| NP_35 | TVAAP | 40 |
| NP_36 | ASTKGP | 41 |
| NP_37 | QPKAAPSVTLFPP | 42 |
| NP_38 | TVAAPSVFIFPP | 43 |
| NP_39 | ASTKGPSVFPLAP | 44 |
| NP_1 | AQGTLSPADKTNVKAAWGKVMT | 45 |
| NP_21 | PGNPTTTVVPPASTSTSRPTSSTSSPVSTPTGQPGG | 46 |
| 1EI1A | GGKFDDNSYKVSGGLHGVG | 47 |
| 1KQFA | GRLFAINKMAEGPFPEHYEPIETPLGTNPLHPNVVSNPVVRLYE | 48 |
| 1B8TA | YGPKGKGKGMGAGTLSTDKGESLGIKYEEGQSHRPTNPNASRMA | 49 |
| | QKVGGSD | |
| JP1 | GKGKG | 50 |
| JP2 | GKGKGMGAG | 51 |
| JP3 | GKGKGMGAGTLSTDKG | 52 |
| JP4 | GKGKGMGAGTLSTDKGESLG | 53 |
| 2ZT5A | KPLKEPKTVNVVQFEPSKGAIGKAYKKDAKLVMEYLAICDECYITE | 54 |
| | MEMLLNEKGEFTIETEGKTFQLTKDMINVKRFQKTLYVE | |
| LP1 | IEGRMD | 55 |
| pVT197 | GGGGSGGGGSGGGGSAAQPA | 56 |
| pVT311 | GGGGSGGGGSGGGGSAAQPAS | 57 |
| LP1 | GGGGSLVPRGSGGGGS | 58 |
| LP2 | GGGGSLVPRGSGGGG | 59 |
| LP3 | GGSGGHMGSGG | 60 |
| LP4 | GGSGGSGGSGG | 61 |
| LP5 | GGSGG | 62 |
| LP6 | GGSGGGGG | 63 |
| LP7 | GGGSEGGGSEGGGSEGGG | 64 |
| LP8 | GSGGGTGGGSG | 65 |
| LP9 | GT | 66 |
| LP10 | GSGSGS | 67 |
| LP11 | GSGSGSGS | 68 |
| LP12 | GSGSGSGSGS | 69 |
| LP13 | GSGSGSGSGSGS | 70 |
| LP14 | GSGSGSGSGSGSGS | 71 |
| LP15 | GSGSGSGSGSGSGSGS | 72 |
| LP16 | SGGSGGSSHS | 73 |
| LP17 | AAGAATAA | 74 |
| LP18 | EPKSADKTHTAPPAP | 75 |
| JP5 | EPKSsDKTHTsPPsP | 76 |
| JP6 | EPKSCDKTHTCPPCP | 77 |
| JP7 | EPKSCDKTHTCPPCPAPELLGGP | 78 |
| JP8 | EPKSsDKTHTsPPsPAPELLGGP | 79 |
| JP9 | EPKSCDKTHTCPPCPAPEaaGGP | 80 |
| JP10 | EPKSsDKTHTsPPsPAPEaaGGP | 81 |
| JP11 | ERKssVEsPPsP | 82 |
| JP12 | ERKCCVECPPCP | 83 |
| JP13 | ERKCCVECPPCPAPPVAGP | 84 |
| JP14 | ERKssVEsPPsPAPPVAGP | 85 |
| JP15 | ELKTPLGDTTHTCPRCPEPKSCDTPPPCPRCPEPKSCDTPPPCPRCPE | 86 |
| | PKSCDTPPPCP | |
| JP16 | ELKTPLGDTTHTsPRsPEPKSsDTPPPsPRsPEPKSsDTPPPsPRsPEPKSs | 87 |
| | DTPPPsP | |
| NP_8 | ELKTPLGDTTHT | 88 |
| JP17 | ESKYGPPsPSsP | 89 |
| JP18 | ESKYGPPCPSCP | 90 |
| JP19 | ESKYGPPCPSCPAPEFLGGP | 91 |
| JP20 | ESKYGPPsPSsPAPEFLGGP | 92 |
| JP21 | ESKYGPPCPSCPAPEaaGGP | 93 |
| JP22 | ESKYGPPsPSsPAPEaaGGP | 94 |
| JP23 | SGSETPGTSESATPEGGSGGS | 95 |
| JP24 | SGSETPGTSESATPES | 96 |
| JP25 | SGSETPGTSES | 97 |

TABLE 4
Useful Constitutive Promoters

| Constitutive | | SEQ ID |
|---|---|---|
| Promoter | Sequence | NO |
| EF1A | GGCTCCGGTGCCCGTCAGTGGGCAGAGCGCACATCGCC | 98 |
| | CACAGTCCCCGAGAAGTTGGGGGGAGGGGTCGGCAATT | |
| | GAACCGGTGCCTAGAGAAGGTGGCGCGGGGTAAACTG | |
| | GGAAAGTGATGTCGTGTACTGGCTCCGCCTTTTTCCCGA | |
| | GGGTGGGGGAGAACCGTATATAAGTGCAGTAGTCGCCG | |
| | TGAACGTTCTTTTTCGCAACGGGTTTGCCGCCAGAACAC | |
| | AGGTAAGTGCCGTGTGTGGTTCCCGCGGGCCTGGCCTCT | |
| | TTACGGGTTATGGCCCTTGCGTGCCTTGAATTACTTCCA | |
| | CCTGGCTGCAGTACGTGATTCTTGATCCCGAGCTTCGGG | |
| | TTGGAAGTGGGTGGGAGAGTTCGAGGCCTTGCGCTTAA | |
| | GGAGCCCCTTCGCCTCGTGCTTGAGTTGAGGCCTGGCCT | |
| | GGGCGCTGGGGCCGCCGCGTGCGAATCTGGTGGCACCT | |
| | TCGCGCCTGTCTCGCTGCTTTCGATAAGTCTCTAGCCAT | |
| | TTAAAATTTTTGATGACCTGCTGCGACGCTTTTTTTCTG | |
| | GCAAGATAGTCTTGTAAATGCGGGCCAAGATCTGCACA | |
| | CTGGTATTTCGGTTTTTGGGGCCGCGGGCGGCGACGGG | |
| | GCCCGTGCGTCCCAGCGCACATGTTCGGCGAGGCGGGG | |
| | CCTGCGAGCGCGGCCACCGAGAATCGGACGGGGGTAGT | |
| | CTCAAGCTGGCCGGCCTGCTCTGGTGCCTGGTCTCGCGC | |
| | CGCCGTGTATCGCCCCGCCCTGGGCGGCAAGGCTGGCC | |
| | CGGTCGGCACCAGTTGCGTGAGCGGAAAGATGGCCGCT | |
| | TCCCGGCCCTGCTGCAGGGAGCTCAAAATGGAGGACGC | |
| | GGCGCTCGGGAGAGCGGGCGGGTGAGTCACCCACACA | |
| | AAGGAAAAGGGCCTTTCCGTCCTCAGCCGTCGCTTCAT | |
| | GTGACTCCACGGAGTACCGGGCGCCGTCCAGGCACCTC | |
| | GATTAGTTC | |
| UBC | GGTGCAGCGGCCTCCGCGCCGGGTTTTGGCGCCTCCCG | 99 |
| | CGGGCGCCCCCCTCCTCACGGCGAGCGCTGCCACGTCA | |
| | GACGAAGGGCGCAGGAGCGTTCCTGATCCTTCCGCCCG | |
| | GACGCTCAGGACAGCGGCCCGCTGCTCATAAGACTCGG | |
| | CCTTAGAACCCCAGTATCAGCAGAAGGACATTTTAGGA | |
| | CGGGACTTGGGTGACTCTAGGGCACTGGTTTTCTTTCCA | |
| | GAGAGCGGAACAGGCGAGGAAAAGTAGTCCCTTCTCGG | |
| | CGATTCTGCGGAGGGATCTCCGTGGGGCGGTGAACGCC | |
| | GATGATTATATAAGGACGCGCCGGGTGTGGCACAGCTA | |
| | GTTCCGTCGCAGCCGGGATTTGGGTCGCGGTTCTTGTTT | |
| | GTGGATCGCTGTGATCGTCACTTGGTGAGTTGCGGGCTG | |
| | CTGGGCTGGCCGGGGCTTTCGTGGCCGCCGGGCCGCTC | |
| | GGTGGGACGGAAGCGTGTGGAGAGACCGCCAAGGGCT | |
| | GTAGTCTGGGTCCGCGAGCAAGGTTGCCCTGAACTGGG | |
| | GGTTGGGGGGAGCGCACAAAATGGCGGCTGTTCCCGAG | |
| | TCTTGAATGGAAGACGCTTGTAAGGCGGGCTGTGAGGT | |
| | CGTTGAAACAAGGTGGGGGGCATGGTGGGCGGCAAGA | |
| | ACCCAAGGTCTTGAGGCCTTCGCTAATGCGGGAAAGCT | |
| | CTTATTCGGGTGAGATGGGCTGGGGCACCATCTGGGGA | |
| | CCCTGACGTGAAGTTTGTCACTGACTGGAGAACTCGGG | |
| | TTTGTCGTCTGGTTGCGGGGGCGGCAGTTATGCGGTGCC | |
| | GTTGGGCAGTGCACCCGTACCTTTGGGAGCGCGCGCCT | |
| | CGTCGTGTCGTGACGTCACCCGTTCTGTTGGCTTATAAT | |
| | GCAGGGTGGGGCCACCTGCCGGTAGGTGTGCGGTAGGC | |
| | TTTTCTCCGTCGCAGGACGCAGGGTTCGGGCCTAGGGT | |
| | AGGCTCTCCTGAATCGACAGGCGCCGGACCTCTGGTGA | |
| | GGGGAGGGATAAGTGAGGCGTCAGTTTCTTTGGTCGGT | |
| | TTTATGTACCTATCTTCTTAAGTAGCTGAAGCTCCGGTT | |
| | TTGAACTATGCGCTCGGGGTTGGCGAGTGTGTTTTGTGA | |
| | AGTTTTTTAGGCACCTTTTGAAATGTAATCATTTGGGTC | |
| | AATATGTAATTTTCAGTGTTAGACTAGTAAA | |
| Pgk | TTCTACCGGGTAGGGGAGGCGCTTTTCCCAAGGCAGTC | 100 |
| | TGGAGCATGCGCTTTAGCAGCCCCGCTGGGCACTTGGC | |
| | GCTACACAAGTGGCCTCTGGCCTCGCACACATTCCACAT | |
| | CCACCGGTAGGCGCCAACCGGCTCCGTTCTTTGGTGGCC | |
| | CCTTCGCGCCACCTTCTACTCCTCCCCTAGTCAGGAAGT | |
| | TCCCCCCCGCCCCGCAGCTCGCGTCGTGCAGGACGTGA | |
| | CAAATGGAAGTAGCACGTCTCACTAGTCTCGTGCAGAT | |
| | GGACAGCACCGCTGAGCAATGGAAGCGGGTAGGCCTTT | |
| | GGGGCAGCGGCCAATAGCAGCTTTGCTCCTTCGCTTTCT | |
| | GGGCTCAGAGGCTGGGAAGGGGTGGGTCCGGGGGCGG | |
| | GCTCAGGGGCGGGCTCAGGGGCGGGGCGGGCGCCCGA | |
| | AGGTCCTCCGGAGGCCCGGCATTCTGCACGCTTCAAAA | |
| | GCGCACGTCTGCCGCGCTGTTCTCCTCTTCCTCATCTCC | |
| | GGGCCTTTCGACCT | |
| CMV | TAGTTATTAATAGTAATCAATTACGGGGTCATTAGTTCA | 101 |
| | TAGCCCATATATGGAGTTCCGCGTTACATAACTTACGGT | |
| | AAATGGCCCGCCTGGCTGACCGCCCAACGACCCCCGCC | |
| | CATTGACGTCAATAATGACGTATGTTCCCATAGTAACGC | |
| | CAATAGGGACTTTCCATTGACGTCAATGGGTGGAGTAT | |
| | TTACGGTAAACTGCCCACTTGGCAGTACATCAAGTGTAT | |
| | CATATGCCAAGTACGCCCCCTATTGACGTCAATGACGG | |
| | TAAATGGCCCGCCTGGCATTATGCCCAGTACATGACCTT | |
| | ATGGGACTTTCCTACTTGGCAGTACATCTACGTATTAGT | |
| | CATCGCTATTACCATGGTGATGCGGTTTTGGCAGTACAT | |
| | CAATGGGCGTGGATAGCGGTTTGACTCACGGGGATTTC | |
| | CAAGTCTCCACCCCATTGACGTCAATGGGAGTTTGTTTT | |
| | GGCACCAAAATCAACGGGACTTTCCAAAATGTCGTAAC | |
| | AACTCCGCCCCATTGACGCAAATGGGCGGTAGGCGTGT | |
| | ACGGTGGGAGGTCTATATAAGCAGAGCTGGTTTAGTGA | |
| | ACCGTCAGATC | |
| CAGGS | ACTAGTTATTAATAGTAATCAATTACGGGGTCATTAGTT | 102 |
| | CATAGCCCATATATGGAGTTCCGCGTTACATAACTTACG | |
| | GTAAATGGCCCGCCTGGCTGACCGCCCAACGACCCCCG | |
| | CCCATTGACGTCAATAATGACGTATGTTCCCATAGTAAC | |
| | GCCAATAGGGACTTTCCATTGACGTCAATGGGTGGAGT | |
| | ATTTACGGTAAACTGCCCACTTGGCAGTACATCAAGTGT | |
| | ATCATATGCCAAGTACGCCCCCTATTGACGTCAATGAC | |
| | GGTAAATGGCCCGCCTGGCATTATGCCCAGTACATGAC | |
| | CTTATGGGACTTTCCTACTTGGCAGTACATCTACGTATT | |
| | AGTCATCGCTATTACCATGGTCGAGGTGAGCCCCACGTT | |
| | CTGCTTCACTCTCCCCATCTCCCCCCCCTCCCCACCCCC | |
| | AATTTTGTATTTATTTATTTTTTAATTATTTTGTGCAGCG | |
| | ATGGGGGCGGGGGGGGGGGGGGGGCGCGCGCCAGGCG | |
| | GGGCGGGGCGGGGCGAGGGGCGGGGCGGGGCGAGGCG | |
| | GAGAGGTGCGGCGGCAGCCAATCAGAGCGGCGCGCTCC | |
| | GAAAGTTTCCTTTTATGGCGAGGCGGCGGCGGCGGCGG | |
| | CCCTATAAAAAGCGAAGCGCGCGGCGGGCGGGGAGTC | |
| | GCTGCGACGCTGCCTTCGCCCCGTGCCCCGCTCCGCCGC | |
| | CGCCTCGCGCCGCCCGCCCCGGCTCTGACTGACCGCGTT | |
| | ACTCCCACAGGTGAGCGGGCGGGACGGCCCTTCTCCTC | |
| | CGGGCTGTAATTAGCGCTTGGTTTAATGACGGCTTGTTT | |
| | CTTTTCTGTGGCTGCGTGAAAGCCTTGAGGGGCTCCGGG | |
| | AGGGCCCTTTGTGCGGGGGGAGCGGCTCGGGGGGTGCG | |
| | TGCGTGTGTGTGTGCGTGGGGAGCGCCGCGTGCGGCTC | |
| | CGCGCTGCCCGGCGGCTGTGAGCGCTGCGGGCGCGGCG | |
| | CGGGGCTTTGTGCGCTCCGCAGTGTGCGCGAGGGGAGC | |
| | GCGGCCGGGGGCGGTGCCCCGCGGTGCGGGGGGGGCT | |
| | GCGAGGGGAACAAAGGCTGCGTGCGGGGTGTGTGCGTG | |
| | GGGGGGTGAGCAGGGGGTGTGGGCGCGTCGGTCGGGCT | |
| | GCAACCCCCCCTGCACCCCCCTCCCCGAGTTGCTGAGCA | |
| | CGGCCCGGCTTCGGGTGCGGGGCTCCGTACGGGGCGTG | |
| | GCGCGGGGCTCGCCGTGCCGGGCGGGGGGTGGCGGCA | |
| | GGTGGGGGTGCCGGGCGGGGCGGGGCCGCCTCGGGCC | |
| | GGGGAGGGCTCGGGGGAGGGGCGCGGCGGCCCCCGGA | |
| | GCGCCGGCGGCTGTCGAGGCGCGGCGAGCCGCAGCCAT | |
| | TGCCTTTTATGGTAATCGTGCGAGAGGGCGCAGGGACT | |
| | TCCTTTGTCCCAAATCTGTGCGGAGCCGAAATCTGGGA | |
| | GGCGCCGCCGCACCCCCTCTAGCGGGCGCGGGGCGAAG | |
| | CGGTGCGGCGCCGGCAGGAAGGAAATGGGCGGGGAGG | |
| | GCCTTCGTGCGTCGCCGCGCCGCCGTCCCCTTCTCCCTC | |
| | TCCAGCCTCGGGGCTGTCCGCGGGGGGACGGCTGCCTT | |
| | CGGGGGGGACGGGGCAGGGCGGGGTTCGGCTTCTGGCG | |
| | TGTGACCGGCGGCTCTAGAGCCTCTGCTAACCATGTTCA | |
| | TGCCTTCTTCTTTTTCCTACAGCTCCTGGGCAACGTGCT | |
| | GGTTATTGTGCTGTCTCATCATTTTGGCAAAGAATTC | |
| SV40 | CTGTGGAATGTGTGTCAGTTAGGGTGTGGAAAGTCCCC | 103 |
| | AGGCTCCCCAGCAGGCAGAAGTATGCAAAGCATGCATC | |
| | TCAATTAGTCAGCAACCAGGTGTGGAAAGTCCCCAGGC | |
| | TCCCCAGCAGGCAGAAGTATGCAAAGCATGCATCTCAA | |
| | TTAGTCAGCAACCATAGTCCCGCCCCTAACTCCGCCCAT | |
| | CCCGCCCCTAACTCCGCCCAGTTCCGCCCATTCTCCGCC | |
| | CCATGGCTGACTAATTTTTTTTATTTATGCAGAGGCCGA | |
| | GGCCGCCTCTGCCTCTGAGCTATTCCAGAAGTAGTGAG | |
| | GAGGCTTTTTTGGAGGCCTAGGCTTTTGCAAAAAGCT | |
| COPIA | ATCTGTTGGAATATACTATTCAACCTACAAAAATAACGT | 104 |
| | TAAACAACACTACTTTATATTTGATATGAATGGCCACAC | |
| | CTTTTATGCCATAAAACATATTGTAAGAGAATACCACTC | |
| | TTTTTATTCCTTCTTTCCTTCTTGTACGTTTTTTGCTGTGA | |
| | GTAGGTCGTGGTGCTGGTGTTGCAGTTGAAATAACTTA | |
| | AAATATAAATCATAAAACTCAAACATAAACTTGACTAT | |
| | TTATTTATTTATTAAGAAAGGAAATATAAATTATAAATT | |
| | ACAACAGGTT | |
| ACT5C | GGAAGTACACTCTTCATGGCGATATACAAGACACACAC | 105 |
| | AAGCACGAACACCCAGTTGCGGAGGAAATTCTCCGTAA | |
| | ATGAAAACCCAATCGGCGAACAATTCATACCCATATAT | |
| | GGTAAAAGTTTTGAACGCGACTTGAGAGCGGAGAGCAT | |
| | TGCGGCTGATAAGGTTTTAGCGCTAAGCGGGCTTTATA | |
| | AAACGGGCTGCGGGACCAGTTTTCATATCACTACCGTTT | |
| | GAGTTCTTGTGCTGTGTGGATACTCCTCCCGACACAAAG | |
| | CCGCTCCATCAGCCAGCAGTCGTCTAATCCAGAGACAC | |
| | C | |
| TRE | GGTACCGAGCTCGACTTTCACTTTTCTCTATCACTGATA | 106 |
| | GGGAGTGGTAAACTCGACTTTCACTTTTCTCTATCACTG | |
| | ATAGGGAGTGGTAAACTCGACTTTCACTTTTCTCTATCA | |
| | CTGATAGGGAGTGGTAAACTCGACTTTCACTTTTCTCTA | |
| | TCACTGATAGGGAGTGGTAAACTCGACTTTCACTTTTCT | |
| | CTATCACTGATAGGGAGTGGTAAACTCGACTTTCACTTT | |
| | TCTCTATCACTGATAGGGAGTGGTAAACTCGACTTTCAC | |
| | TTTTCTCTATCACTGATAGGGAGTGGTAAACTCGACCTA | |
| | TATAAGCAGAGCTCGTTTAGTGAACCGTCAGATCGCCT | |
| | GGAGACGCCATCCACGCTGTTTTGACCTCCATAGAAGA | |
| | CACCGGGACCGATCCAGCCTCCGCGGCCCCGAATTG | |

All headings and section designations are used for clarity and reference purposes only and are not to be considered limiting in any way. For example, those of skill in the art will appreciate the usefulness of combining various aspects from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method for controlling the immunogenicity of an engineered cell, the method comprising:
(a) obtaining an isolated cell;
(b) introducing into the isolated cell (i) a nucleic acid comprising an inducible RNA polymerase promoter operably linked to an shRNA sequence targeting an immunosuppressive factor and (ii) a nucleic acid comprising a promoter operably linked to a transactivator element corresponding to the inducible RNA polymerase promoter to produce an engineered cell; and
(c) exposing the engineered cell to an exogenous factor to activate the transactivator element, thereby controlling the immunogenicity of the engineered cell.

2. A method for controlling the immunogenicity of an engineered cell, the method comprising:
(a) obtaining an isolated cell;
(b) introducing into the isolated cell a nucleic acid comprising (i) a sequence encoding an inducible degron element operably linked to an immunosuppressive factor or (ii) a sequence encoding an immunosuppressive factor operably linked to an inducible degron element to produce an engineered cell; and
(c) exposing the engineered cell to an exogenous factor to activate the inducible degron element, thereby controlling the immunogenicity of the engineered cell.

3. A method for controlling immunogenicity of an engineered cell comprising:
(a) obtaining an isolated cell;
(b) introducing into the isolated cell:
(i) a first construct comprising from 5′ end to 3′ end: a first promoter and an immunosuppressive factor gene;
(ii) a second construct comprising from 5′ end to 3′ end: a second promoter and a nucleic acid sequence encoding Cas9 or a variant thereof; and
(ii) a third construct comprising from 5′ end to 3⁻ end: an inducible RNA polymerase promoter, a guide RNA (gRNA) sequence targeting the immunosuppressive factor, a third promoter, and a transactivator element corresponding to the inducible RNA polymerase promoter; and
(c) exposing the engineered cell to an exogenous factor to activate the transactivator element, thereby controlling the immunogenicity of the engineered cell.

4. A method for controlling the immunogenicity of an engineered cell, the method comprising:
(a) obtaining an isolated cell;
(b) introducing into the isolated cell (i) a nucleic acid comprising an inducible RNA polymerase promoter operably linked to an immune signaling factor gene and (ii) a nucleic acid comprising a promoter operably linked to a transactivator element corresponding to the inducible RNA polymerase promoter to produce an engineered cell; and
(c) exposing the engineered cell to an exogenous factor to activate the transactivator element, thereby controlling the immunogenicity of the engineered cell.

5. The method of any one of claims 1-4, further comprising administering the engineered cell to a subject prior to step (c).

6. The method of claim 1 or 5, wherein step (b) comprises introducing into the isolated cell a single nucleic acid construct comprising (i) the inducible RNA polymerase promoter operably linked the shRNA sequence targeting the immunosuppressive factor and (ii) the promoter operably linked to the transactivator element.

7. The method of claim 6, wherein the construct comprises from 5′ end to 3′ end: the inducible RNA polymerase promoter; the shRNA sequence; the promoter; and the transactivator element.

8. The method of claim 4 or 5, wherein step (b) comprises introducing into the isolated cell a single nucleic acid construct comprising (i) the inducible RNA polymerase promoter operably linked the immune signaling factor gene and (ii) the promoter operably linked to the transactivator element.

9. The method of any one of claim 4, 5, or 8, wherein the construct comprises from 5′ end to 3′ end: the inducible RNA polymerase promoter, the immune signaling factor gene, the promoter, and the transactivator element.

10. The method of any one of claim 1 or 5-7, wherein the isolated cell is engineered to exogenously express the immunosuppressive factor.

11. The method of any one of claim 1 or 5-7 or 10, wherein the isolated cell overexpresses the immunosuppressive factor in the absence of the exogenous factor that activates the transactivator element.

12. The method of any one of claim 1 or 5-7 or 10-11, wherein the inducible RNA polymerase promoter is a U6Tet promoter.

13. The method of claim 3 or 5, wherein the inducible RNA polymerase promoter is U6Tet promoter, the transactivator element is a Tet Repressor element, and the exogenous factor is tetracycline or a derivative thereof.

14. The method of any one of claim 4, 5, or 8-9, wherein the inducible RNA polymerase promoter is a TRE promoter and the transactivator element is a Tet-On element, and the exogenous factor is tetracycline or a derivative thereof.

15. The method of claim 2 or 5, wherein a flexible linker connects the inducible degron element to the immunosuppressive factor.

16. The method of claim 15, wherein the flexible linker is selected from the group consisting of (GSG)ₙ(SEQ ID NO:3), (GGGS)ₙ (SEQ ID NO:1), and (GGGSGGGS)ₙ (SEQ ID NO:2), wherein n is 1-10.

17. The method of any one of claim 1, 5, or 15-16, wherein step (b) comprises introducing into the isolated cell a single nucleic acid construct comprising a promoter operably linked to the nucleic acid.

18. The method of any one of claim 1-4 or 17, wherein the promoter is a constitutive promoter selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

19. The method of any one of claim 3, 5, or 13, wherein the first, second and/or third promoters are constitutive promoters, each independently selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

20. The method of any one of claim 1-3 or 5-19, wherein the immunosuppressive factor is selected from the group consisting of CD47, CD24, CD200, HLA-G, HLA-E, HLA-C, HLA-E heavy chain, PD-L1, IDO1, CTLA4-Ig, C1-Inhibitor, IL-10, IL-35, FASL, Serpinb9, CCl21, and Mfge8.

21. The method of any one of claim 1, 5-14, or 17-20, wherein the construct comprises from 5′ end to 3′ end: a U6Tet promoter, a shRNA sequence targeting CD47, an EF1a promoter, and a Tet Repressor element, and wherein the exogenous factor is tetracycline or a derivative thereof.

22. The method of any one of claim 1, 6-14, or 17-20, wherein the construct further comprises a vector backbone for lentiviral expression.

23. The method of any one of claim 2, 5, or 8-20, wherein the inducible degron element is selected from the group consisting of a ligand inducible degron element, a peptidic degron element, and a peptidic proteolysis targeting chimera (PROTAC) element.

24. The method of claim 23, wherein the ligand inducible degron element is selected from a small molecule-assisted shutoff (SMASH) degron element, Shield-1 responsive degron element, auxin responsive degron element, and a rapamycin responsive degron element.

25. The method of claim 23 or 24, wherein the ligand inducible degron element is a small molecule-assisted shutoff (SMASH) degron element and the exogenous factor is asunaprevir.

26. The method of any one of claim 17-18, 20, or 23-25, wherein the construct further comprises a 5′ homology arm and a 3′ homology arm for targeted integration to a safe harbor locus selected from the group consisting of an AAVS1 locus, a CLBYL locus, a CXCR4 locus, a Rosa26 locus, and a CCR5 locus.

27. The method of any one of claims 1-4 and 5-26, wherein the isolated cell is an isolated human cell further comprising deletion or reduced expression of MHC class I human leukocyte antigens and/or deletion or reduced expression of MHC class II human leukocyte antigens compared to an unmodified human cell.

28. The method of any one of claims 1-4 and 5-27, wherein the isolated human cell further comprises deletion or reduced expression of CIITA, B2M, and/or NLRC5.

29. The method of any one of claims 1-4 and 5-28, wherein the isolated human cell is hypoimmunogenic and either a stem cell or a differentiated cell thereof; wherein the stem cell is selected from the group consisting of an embryonic stem cell, a pluripotent stem cell, an induced pluripotent stem cell, and an adult stem cell, and wherein the differentiated cell is selected from the group consisting of a cardiac cell, liver cell, kidney cell, pancreatic cell, neural cell, immune cell, mesenchymal cell, and endothelial cell.

30. The method of claim 29, wherein the differentiated cell is a pancreatic cell.

31. A construct comprising from 5′ end to 3′ end: an inducible RNA polymerase promoter; an shRNA sequence targeting an immunosuppressive factor; a constitutive promoter; and a transactivator element corresponding to the inducible RNA polymerase promoter.

32. A construct comprising from 5′ end to 3′ end: an inducible RNA polymerase promoter; an immune signaling factor gene; a promoter; and a transactivator element corresponding to the inducible RNA polymerase promoter.

33. The construct of claim 31, wherein the inducible RNA polymerase promoter is a U6Tet promoter.

34. The construct of claim 32, wherein the inducible RNA polymerase promoter is a TRE promoter.

35. The construct of claim 31 or 33, wherein the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

36. The construct of claim 32 or 34, wherein the immune signaling factor is selected from the group consisting of B2M, MIC-A, MIC-B, HLA-A, HLA-B, HLA-C, RFXANK, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

37. The construct of any one of claims 31-36, wherein the constitutive promoter is selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

38. The construct of any one of claim 31, 33, 35, or 37, comprises from 5′ end to 3′ end: a U6Tet promoter, a shRNA sequence targeting CD47, an EF1a promoter, and a Tet Repressor element.

39. The construct of any one of claim 32, 34, 36, or 37, comprises from 5′ end to 3′ end: a TRE promoter, an immune signaling factor gene, an EF1a promoter, and a Tet-On element.

40. The construct of any one of claims 31-39, further comprising a vector backbone for lentiviral expression.

41. A composition comprising an isolated cell comprising a construct of any one of claims 31-40.

42. A composition comprising an isolated cell comprising a construct of any one of claim 31, 33, or 35-38, wherein the isolated cell is engineered to exogenously express the immunosuppressive factor.

43. The composition of claim 42, wherein the isolated cell overexpresses the immunosuppressive factor in the absence of the exogenous factor that activates the transactivator element.

44. The composition of any one of claims 40-43, wherein the isolated cell is exposed to an exogenous factor to activate the transactivator element.

45. The composition of any one of claims 41-44, wherein the isolated cell is a stem cell selected from the group consisting of an embryonic stem cell, a pluripotent stem cell, and an adult stem cell.

46. A composition comprising isolated differentiated cells prepared by culturing the stem cell of claim 45 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

47. A method of treating a patient in need of cell therapy comprising:
(a) administering the composition of claim 46 to a patient; and
(b) exposing the composition to an exogenous factor to activate the inducible RNA polymerase promoter, thereby controlling immunogenicity of the cells of the composition.

48. A pluripotent stem cell comprising (i) reduced or silenced expression of MHC class I molecules and/or MHC class II molecules, (ii) overexpression of CD47, and (iii) a factor selected from the group consisting of: an inducible shRNA targeting CD47, an inducible degron element controlling CD47, or a SMASH degron element controlling CD47.

49. A pluripotent stem cell comprising (i) reduced or silenced expression of B2M and CIITA, (ii) overexpression of CD47, and (iii) a factor selected from the group consisting of: an inducible shRNA targeting CD47, an inducible degron element controlling CD47, or a SMASH degron element controlling CD47.

50. A pluripotent stem cell comprising (i) reduced or silenced expression of MHC class I molecules and/or MHC class II molecules, (ii) overexpression of CD47, (iii) a Cas9 or a variant thereof, and (iv) an inducible guide RNA targeting CD47.

51. A pluripotent stem cell comprising (i) reduced or silenced expression of B2M and CIITA, (ii) overexpression of CD47, (iii) a Cas9 or a variant thereof, and (iv) an inducible guide RNA targeting CD47.

52. A pluripotent stem cell comprising (i) reduced or silenced expression of MHC class I molecules and/or MHC class II molecules, (ii) overexpression of CD47, and (iii) an inducible protein degradation system for modulating expression of CD47 selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

53. A pluripotent stem cell comprising (i) reduced or silenced expression of B2M and CIITA, (ii) overexpression of CD47, and (iii) an inducible protein degradation system for modulating expression of CD47 selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

54. A pluripotent stem cell comprising (i) reduced or silenced expression of MHC class I molecules and/or MHC class II molecules, (ii) overexpression of CD47, and (iii) an RNA regulation system for modulating expression of CD47 selected from the group consisting of an inducible shRNA, an inducible siRNA, a CRISPR interference (CRISPRi), and a RNA targeting nuclease system.

55. A pluripotent stem cell comprising (i) reduced or silenced expression of B2M and CIITA, (ii) overexpression of CD47, and (iii) an RNA regulation system for modulating expression of CD47 selected from the group consisting of an inducible shRNA, an inducible siRNA, a CRISPR interference (CRISPRi), and a RNA targeting nuclease system.

56. A pluripotent stem cell comprising (i) reduced or silenced expression of MHC class I molecules and/or MHC class II molecules, (ii) overexpression of CD47, and (iii) a DNA regulation system for modulating expression of CD47 selected from the group consisting of a tissue specific promoter expression system, an inducible promoter expression system, a molecule regulated riboswitch system, and an inducible nuclease-based genome editing system.

57. A pluripotent stem cell comprising (i) reduced or silenced expression of B2M and CIITA, (ii) overexpression of CD47, and (iii) a DNA regulation system for modulating expression of CD47 selected from the group consisting of a tissue specific promoter expression system, an inducible promoter expression system, a molecule regulated riboswitch system, and an inducible nuclease-based genome editing system.

58. A pluripotent stem cell comprising (i) reduced or silenced expression of MHC class I molecules and/or MHC class II molecules, (ii) overexpression of CD47, and (iii) an inducible system for modulating expression of CD47.

59. A pluripotent stem cell comprising (i) reduced or silenced expression of B2M and CIITA, (ii) overexpression of CD47, and (iii) an inducible system for modulating expression of CD47.

60. A differentiated cell derived from the pluripotent stem cell of any one of claims 48-59, wherein the differentiated cell is selected from the group consisting of a cardiac cell, liver cell, kidney cell, pancreatic cell, neural cell, immune cell, mesenchymal cell, and endothelial cell.

61. A construct comprising from 5′ end to 3′ end: a promoter, an inducible degron element, an optional sequence encoding a flexible linker, and an immunosuppressive factor gene.

62. A construct comprising from 5′ end to 3′ end: a promoter, an immunosuppressive factor gene, an optional sequence encoding a flexible linker, and an inducible degron element.

63. The construct of claim 61 or 62, wherein the promoter is a constitutive promoter selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

64. The construct of any one of claims 61-63, wherein the flexible linker is selected from the group consisting of (GSG)ₙ(SEQ ID NO:3), (GGGS)ₙ (SEQ ID NO:1), and (GGGSGGGS)ₙ (SEQ ID NO:2), wherein n is 1-10.

65. The construct of any one of claims 61-64, wherein the immunosuppressive factor is selected from the group consisting of CD47, CD24, CD200, HLA-G, HLA-E, HLA-C, HLA-E heavy chain, PD-L1, IDO1, CTLA4-Ig, C1-Inhibitor, IL-10, IL-35, FASL, Serpinb9, CCl21, and Mfge8.

66. The construct of any one of claims 61-65, wherein the inducible degron element is selected from the group consisting of a ligand inducible degron element, an inducible peptidic degron element, and a peptidic proteolysis targeting chimera (PROTAC) element.

67. The construct of claim 66, wherein the ligand inducible degron element is selected from a small molecule-assisted shutoff (SMASH) degron element, Shield-1 responsive degron element, auxin responsive degron element, and rapamycin responsive degron element.

68. The construct of any one of claims 61-67, further comprising a 5′ homology arm and a 3′ homology arm for targeted integration to a genomic safe harbor locus selected from the group consisting of an AAVS1 locus, a CLBYL locus, a CXCR4 locus, a Rosa26 locus, and a CCR5 locus.

69. A composition comprising an isolated cell comprising a construct of any one of claims 61-68.

70. The composition of claim 69, wherein the isolated cell is a stem cell selected from the group consisting of a stem cell, an embryonic stem cell, a pluripotent stem cell, and an adult stem cell.

71. A composition comprising isolated differentiated cells prepared by culturing the stem cell of claim 70 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

72. A method of treating a patient in need of cell therapy comprising:
(a) administering the composition of claim 71 to the patient; and
(b) exposing the composition to an exogenous factor to activate the inducible degron element, thereby controlling immunogenicity of the cells of the composition.

73. A composition comprising an isolated cell comprising a DNA targeted nuclease system for controlling immunogenicity of the cell comprising:
(a) a first element comprising from 5′ end to 3′ end: a first promoter and an immunosuppressive factor gene;
(b) a second element comprising from 5′ end to 3′ end: a second promoter and a nucleic acid sequence encoding Cas9 or a variant thereof; and
(c) a third element comprising from 5′ end to 3′ end: an inducible RNA polymerase promoter, a guide RNA (gRNA) sequence targeting the immunosuppressive factor, a third promoter, and a transactivator element corresponding to the inducible promoter.

74. The composition of claim 73, wherein immunogenicity of the cell is controllable upon exposing the cell to an exogenous factor to induce activity of the transactivator element.

75. The composition of claim 73 or 74, wherein the inducible RNA polymerase promoter is a U6Tet promoter, the transactivator element is a Tet Repressor element, and the exogenous factor is tetracycline or a derivative thereof.

76. The composition of any one of claims 73-75, wherein the immunosuppressive factor is selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

77. The composition of any one of claims 73-76, wherein the first, second and/or third promoters are constitutive promoters, each independently selected from the group consisting of an EF1A promoter, an EFS promoter, a CMV promoter, a CAGGS promoter, a SV40 promoter, a COPIA promoter, an ACT5C promoter, a TRE promoter, a CBh promoter, a PGK promoter, and a UBC promoter.

78. The composition of any one of claims 73-77, wherein the isolated cell is an isolated engineered human cell further comprising deletion or reduced expression of MHC class I human leukocyte antigens and/or deletion or reduced expression of MHC class II human leukocyte antigens compared to an unmodified human cell.

79. The composition of claim 78, wherein the isolated human cell further comprises deletion or reduced expression of CIITA, B2M, and/or NLRC5.

80. The composition of claim 78 or 79, wherein the isolated human cell is hypoimmunogenic and a stem cell.

81. A composition comprising isolated differentiated cells prepared by culturing the stem cell of claim 80 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

82. A method of treating a patient in need of cell therapy comprising:
(a) administering the composition of claim 81; and
(b) exposing the composition to an exogenous factor to activate the inducible RNA polymerase promoter, thereby controlling immunogenicity of the cells of the composition.

83. A composition comprising an isolated mammalian cell comprising a modification comprising a recombinant nucleic acid sequence encoding a system for conditional expression of one or more immunosuppressive factors.

84. A composition comprising an isolated mammalian cell comprising a recombinant nucleic acid sequence encoding a system for conditional expression of one or more immune signaling factors.

85. The composition of claim 83, wherein the expression of the one or more immunosuppressive factors is controllable by an exogenous factor.

86. The composition of claim 84, wherein the expression of the one or more immune signaling factors is controllable by an exogenous factor.

87. The composition of claim 83 or 85, wherein the system comprises an inducible protein degradation system to reduce protein levels of the one or more immunosuppressive factors.

88. The composition of claim 87, wherein the inducible protein degradation system is selected from the group consisting of a small molecule-assisted shutoff (SMASH) system, a Shield-1-inducible degron, an auxin-inducible degron, an IMid-inducible degron, a peptidic degron, a proteolysis targeting chimera, and an antibody for targeted degradation.

89. The composition of claim 83 or 85, wherein the system comprises an RNA regulation system to controllably reduce RNA levels of the one or more immunosuppressive factors.

90. The composition of claim 89, wherein the RNA regulation system is selected from the group consisting of an inducible shRNA, an inducible siRNA, a CRISPR interference (CRISPRi), and an RNA targeting nuclease system.

91. The composition of claim 90, the RNA regulation system is controllable by a ligand inducible transcription factor, a SynNotch receptor, or a ligand regulated riboswitch.

92. The composition of claim 83 or 85, wherein the system comprises a DNA regulation system to reduce expression levels of the one or more immunosuppressive factors that is selected from the group consisting of a tissue-specific promoter expression system, an inducible promoter expression system, a molecule regulated riboswitch system, and an inducible nuclease-based genome editing system.

93. The composition of claim 92, wherein the inducible promoter expression system comprises a U6Tet promoter and a Tet Repressor element.

94. The composition of claim 84 or 86, wherein the system comprises an inducible protein stabilization system to increase protein levels of the one or more immune signaling factors.

95. The composition of claim 94, wherein the inducible protein stabilization system comprises a ligand-inducible protein stabilization system and a small molecule-inducible protein stabilization system.

96. The composition of claim 84 or 86, wherein the system comprises an RNA regulation system to increase RNA levels of the one or more immune signaling factors.

97. The composition of claim 96, wherein the RNA regulation system comprises a CRISPR activation (CRISPRa) system.

98. The composition of claim 84 or 86, wherein the system comprises a DNA regulation system to increase expression levels of the one or more immune signaling factors.

99. The composition of claim 98, wherein the DNA regulation system comprises one or more DNA regulation systems selected from the group consisting of a CRISPR activation (CRISPRa) system, a tissue-specific promoter, an inducible promoter, and a molecule regulated riboswitch system.

100. The composition of claim 92 or 99, wherein the tissue-specific promoter is selected from the group consisting of a cardiac cell-specific promoter, hepatocyte-specific promoter, kidney cell-specific promoter, pancreatic cell-specific promoter, neural cell-specific promoter, immune cell-specific promoter, mesenchymal cell-specific promoter, and endothelial cell-specific promoter.

101. The composition of claim 99, wherein the inducible promoter comprises a TetOn system.

102. The composition of claim 92 or 99, wherein the molecule regulated riboswitch system comprises a theophylline regulated riboswitch or a guanine regulated riboswitch.

103. The composition of claim 92, wherein the inducible nuclease-based genome editing system comprises one selected from the group consisting of CRISPR genome editing comprising an inducible guide RNA targeting the one or more immunosuppressive factors, inducible TALEN genome editing, inducible ZFN genome editing, and small molecule enhanced CRISPR-based genome editing.

104. The composition of any one of claim 83, 85, 87-93, 100, or 102-103, wherein the one or more immunosuppressive factors are selected from the group consisting of CD47, CD24, CD200, HLA-G, HLA-E, HLA-C, HLA-E heavy chain, PD-L1, ID01, CTLA4-Ig, C1-Inhibitor, IL-10, IL-35, FASL, Serpinb9, CCl21, and Mfge8.

105. The composition of any one of claim 84, 86, or 94-102, wherein the one or more immune signaling factors are selected from the group consisting of beta-2-microglobulin (B2M), MHC class I chain-related protein A (MIC-A), MHC class I chain-related protein B (MIC-B), HLA-A, HLA-B, HLA-C, RFXANK, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

106. The composition of any one of claims 83-105, wherein the isolated mammalian cell is an isolated engineered human cell further comprising deletion or reduced expression of MHC class I human leukocyte antigens and/or deletion or reduced expression of MHC class II human leukocyte antigens compared to an unmodified human cell.

107. The composition of claim 106, wherein the isolated engineered human cell further comprises deletion or reduced expression of CIITA, B2M, and/or NLRC5.

108. The composition of any one of claims 106-107, wherein the isolated engineered human cell is hypoimmunogenic and a stem cell.

109. A composition comprising an isolated differentiated cell prepared by culturing the stem cell of claim 108 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

110. A method of treating a patient in need of cell therapy comprising:
(a) administering the composition of claim 109; and
(b) exposing the composition to an exogenous factor to control expression of the one or more immunosuppressive factors, thereby controlling immunogenicity of the cells of the composition.

111. A construct comprising from 5′ to 3′ end: (1) a safety switch transgene; (2) a ribosomal skipping sequence and/or a sequence encoding a linker; (3) a hypoimmunity gene.

112. A construct comprising from 5′ to 3′ end: (1) a hypoimmunity gene; (2) a ribosomal skipping sequence or a linker; (3) a safety switch transgene.

113. The construct of claim 111 or 112, wherein the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

114. The construct of any one of claims 101-103, wherein the ribosomal skipping sequence comprises a sequence encoding an IRES sequence or a sequence encoding a 2A-coding sequence.

115. The construct of any one of claims 111-114, wherein the linker is selected from any one of the linkers provided in Table 3.

116. The construct of any one of claims 111-115, wherein the hypoimmunity gene is selected from the group consisting of: CD47, CD24, CD200, HLA-G, HLA-E, HLA-C, HLA-E heavy chain, PD-L1, IDO1, CTLA4-Ig, IL-10, IL-35, FASL, Serpinb9, CCl21, and Mfge8.

117. The construct of any one of claim 111 or 113-116, further comprising a transcriptional regulatory element operably linked to the safety switch transgene and a polyadenylation sequence at the 3′ end of the hypoimmunity gene, or a transcriptional regulatory element operably linked to the hypoimmunity gene and a polyadenylation sequence at the 3′ end of the safety switch transgene.

118. The construct of any one of claims 111-117, wherein the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

119. The construct of any one of claims 111-118, further comprising a vector backbone for lentiviral expression.

120. A method of delivering a construct into an isolated cell comprising transducing an isolated cell with a lentiviral construct comprising a construct of claim 119; and selecting an engineered cell carrying the safety switch transgene and the hypoimmunity gene.

121. An isolated cell or a population thereof comprising a construct of any one of claims 111-119.

122. The isolated cell or the population thereof of claim 121, wherein the construct has been introduced into a target gene locus.

123. The isolated cell or the population thereof of claim 121 or 122, wherein the target gene locus is either a safe harbor locus selected from the group consisting of an AAVS1 locus, a CLBYL locus, a CXCR4 locus, a Rosa26 locus, and a CCR5 locus, or an immune signaling gene locus selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

124. The isolated cell or the population thereof of any one of claims 121-123, wherein the isolated cell is an isolated engineered human cell further comprising deletion or reduced expression of MHC class I human leukocyte antigens and/or deletion or reduced expression of MHC class II human leukocyte antigens compared to an unmodified human cell.

125. The isolated cell or the population thereof of any one of claims 121-124, wherein the isolated cell further comprises deletion or reduced expression of CIITA, B2M, and/or NLRC5.

126. The isolated cell or the population thereof of any one of claims 121-125, wherein the isolated cell is hypoimmunogenic and a stem cell.

127. A differentiated cell or a population thereof prepared by culturing the stem cell of claim 126 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

128. A method of treating a patient in need of cell therapy comprising administering to patient the differentiated cell or the population thereof of claim 127.

129. A method of treating a patient comprising activating a safety switch in a patient previously administered the differentiated cell or the population thereof of claim 127.

130. A construct for homology directed repair into a safe harbor locus comprising from 5′ to 3′ end: (1) a first homology arm homologous to a first endogenous sequence of a safe harbor locus; (2) a safety switch transgene; (3) a ribosomal skipping sequence and/or a sequence encoding a linker; (4) an hypoimmunity gene; (5) a polyadenylation sequence; and (6) a second homology arm homologous to a second endogenous sequence of the safe harbor locus.

131. A construct for homology directed repair into a safe harbor locus comprising from 5′ to 3′ end: (1) a first homology arm homologous to a first endogenous sequence of an immune signaling gene locus; (2) a safety switch transgene; (3) a ribosomal skipping sequence and/or a sequence encoding a linker; (4) an hypoimmunity gene; (5) a polyadenylation sequence; and (6) a second homology arm homologous to a second endogenous sequence of the immune signaling gene locus.

132. A construct for homology directed repair into a safe harbor locus comprising from 5′ to 3′ end: (1) a first homology arm homologous to a first endogenous sequence of a safe harbor locus; (2) a safety switch transgene; (3) a ribosomal skipping sequence or a sequence encoding a linker; (4) an essential cell factor gene; (5) a polyadenylation sequence; and (6) a second homology arm homologous to a second endogenous sequence of the safe harbor locus.

133. A construct for homology directed repair into an immune signaling comprising from 5′ to 3′ end: (1) a first homology arm homologous to a first endogenous sequence of an immune signaling gene locus; (2) a safety switch transgene; (3) a ribosomal skipping sequence or a sequence encoding a linker; (4) an essential cell factor gene; (5) a polyadenylation sequence; and (6) a second homology arm homologous to a second endogenous sequence of the immune signaling gene locus.

134. A construct for homology directed repair into an essential cell factor gene locus comprising from 5′ to 3′ end: (1) a first homology arm homologous to a first endogenous sequence of an essential cell factor gene locus; (2) a sequence encoding a linker; (3) a safety switch transgene; and (4) a second homology arm homologous to a second endogenous sequence of the essential cell factor gene locus.

135. The construct of claim 130 or 131, wherein the hypoimmunity gene is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, and Mfge8.

136. The construct of claim 132 or 133, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

137. The construct of claim 134, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

138. The construct of any one of claim 130, 132, or 135-136, wherein the safe harbor locus is selected from the group consisting of an AAVS1 locus, a CLBYL locus, a CXCR4 locus, a Rosa26 locus, and a CCR5 locus.

139. The construct of any one of claim 131, 133, or 135-136, wherein the immune signaling gene locus is selected from the group consisting of an B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAE11L/ULBP6, RAET1N/ULBP3, and other ligands of NKG2D.

140. The construct of any one of claim 133, 136, or 139, wherein the immune signaling gene locus is selected from the group consisting of B2M, HLA-A, HLA-B, HLA-C, HLA-D, and HLA-E.

141. The construct of any one of claim 130-133, 135-136, or 138-140, wherein the ribosomal skipping sequence comprises a sequence encoding an IRES sequence or a sequence encoding a 2A-coding sequence.

142. The construct of claim 141, wherein the 2A-coding sequence is selected from the group consisting of T2A, P2A, E2A, and F2A.

143. The construct of any one of claim 130-133, 135-136, or 138-142, wherein the construct enables a targeting nuclease to cleave the safe harbor locus or the immune signaling gene locus, thereby allowing the construct to recombine into the locus by homology directed repair.

144. The construct of any one of claim 134 or 137, wherein the construct enables a targeting nuclease to cleave the essential cell factor gene locus, thereby allowing the construct to recombine into the locus by homology directed repair.

145. The construct of any one of claims 130-144, further comprising a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter located at the 5′ end of the safety switch transgene.

146. The construct of any one of claims 130-145, wherein the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

147. The construct of any one of claims 130-146, wherein the linker is selected from any one of the linkers provided in Table 3.

148. An isolated cell or a population thereof comprising a safety switch transgene and a hypoimmunity gene integrated into a safe harbor locus or an immune signaling gene locus, wherein the construct of any one of claim 130, 135, 138, 141-143, or 145-147 has recombined into the endogenous safe harbor locus of a cell, or wherein the construct of any one of claim 131, 135, or 135-147 has recombined into the endogenous immune signaling gene locus of a cell.

149. An isolated cell or a population thereof comprising a safety switch transgene and an essential cell factor gene integrated into a safe harbor locus or an immune signaling gene locus, wherein the construct of any one of claim 132, 136, 138, 141, 143, or 145-147 has recombined into the endogenous safe harbor locus of a cell, or wherein the construct of any one of claim 133, 136, 139-143, or 145-147 has recombined into the endogenous immune signaling gene locus of a cell, and wherein the cell or the population thereof is unable to express the essential cell factor from the endogenous locus.

150. The isolated cell or the population thereof of claim 148 or 149, wherein the isolated cell is an isolated engineered human cell further comprising deletion or reduced expression of MHC class I human leukocyte antigens and/or deletion or reduced expression of MHC class II human leukocyte antigens compared to an unmodified human cell.

151. The isolated cell or the population thereof of any one of claims 148-150, wherein the isolated cell further comprises deletion or reduced expression of CIITA, B2M and/or NLRC5.

152. The isolated cell or the population thereof of any one of claims 148-151, wherein the isolated cell is hypoimmunogenic and a stem cell.

153. A differentiated cell or a population thereof prepared by culturing the stem cell of claim 152 under differentiation conditions appropriate for differentiation of the stem cell into a cell type selected from the group consisting of cardiac cells, liver cell, kidney cells, pancreatic cells, neural cells, immune cells, mesenchymal cells, and endothelial cells.

154. A method of treating a patient in need of cell therapy comprising administering to a patient the differentiated cell or the population thereof of claim 153.

155. A method of treating a patient comprising activating a safety switch in a patient previously administered the differentiated cell or the population thereof of claim 153.

156. A homology independent donor construct comprising from 5′ to 3′ end: (1) a 5′ long terminal repeats (LTR) comprising a left element (LE); (2) a splice acceptor-viral 2A peptide (SA-2A) element; (3) a safety switch transgene; (4) a ribosomal skipping sequence or sequence encoding a linker; (5) a hypoimmunity gene; (6) a polyadenylation sequence; and (7) 3′ LTR comprising a right element (RE).

157. A homology independent donor construct comprising from 5′ to 3′ end: (1) a 5′ long terminal repeats (LTR) comprising a left element (LE); (2) a splice acceptor-viral 2A peptide (SA-2A) element; (3) a safety switch transgene; (4) a ribosomal skipping sequence or a sequence encoding a linker; (5) an essential cell factor gene; (6) a polyadenylation sequence; and (7) 3′ LTR comprising a right element (RE).

158. A homology independent donor construct comprising from 5′ to 3′ end: (1) a 5′ long terminal repeats (LTR) comprising a left element (LE); (2) a splice acceptor-viral 2A peptide (SA-2A) element; (3) an essential cell factor gene; (4) a ribosomal skipping sequence or a sequence encoding a linker; (5) a safety switch transgene; (6) a polyadenylation sequence; and (7) 3′ LTR comprising a right element (RE).

159. The construct of claim 156, wherein the hypoimmunity gene is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, and Mfge8.

160. The construct of claim 157 or 158, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

161. The construct of any one of claims 156-160, wherein the construct is configured to integrate into a target gene locus of an isolated cell to disrupt expression of the target gene.

162. The construct of any one of claims 156-161, wherein the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

163. The construct of any one of claims 156-162, wherein the target gene locus is an immune signaling gene locus selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

164. The construct of any one of claims 156-162, wherein the target gene locus is an immune signaling gene locus selected from the group consisting of B2M, HLA-A, HLA-B, HLA-C, HLA-D, and HLA-E.

165. The construct of any one of claims 156-164, wherein the target gene locus is a safe harbor locus selected from the group consisting of an AAVS1 locus, a CLBYL locus, a CXCR4 locus, a Rosa26 locus, and a CCR5 locus.

166. An isolated cell or a population thereof comprising the construct of any one of claims 156-165, wherein the construct has integrated into an endogenous target gene to disrupt expression target gene expression in the isolated cell.

167. The isolated cell or the population of claim 166, wherein the isolated cell is unable to express the essential cell factor from the endogenous loci.

168. The isolated cell or the population thereof of claim 167, wherein the construct has integrated into the target gene at a nuclease or transposase target site.

169. The isolated cell or the population thereof of any one of claims 166-168, wherein one allele of the target gene is disrupted by a nuclease or transposase targeting.

170. The isolated cell or the population thereof of any one of claims 166-169, wherein both alleles of the target gene are disrupted by the nuclease or transposase targeting.

171. The isolated cell or the population thereof of any one of claims 166-170, wherein the isolated cell is an isolated engineered human cell further comprising deletion or reduced expression of MHC class I human leukocyte antigens and/or deletion or reduced expression of MHC class II human leukocyte antigens compared to an unmodified human cell.

172. The isolated cell or the population thereof of claim 171, wherein the isolated cell further comprises deletion or reduced expression of CIITA, B2M, and/or NLRC5.

173. The isolated cell or the population thereof of any one of claims 166-172, wherein the isolated cell is hypoimmunogenic and a stem cell.

174. A differentiated cell or a population thereof prepared by culturing the stem cell of claim 173 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

175. A method of treating a patient in need of cell therapy comprising administering to a patient the differentiated cell or the population thereof of claim 174.

176. A method of treating a patient comprising activating the safety switch in the patient previously administered the differentiated cell or the population thereof of claim 174 or 175.

177. An isolated cell or a population thereof comprising an essential cell factor gene operably linked to a sequence encoding a linker that is operably linked to a safety switch transgene.

178. The isolated cell or the population thereof of claim 177, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

179. The isolated cell or the population thereof of claim 177 or 178, wherein the linker is selected from any one of the linkers provided in Table 3.

180. The isolated cell or the population thereof of any one of claims 177-179, wherein the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD30 transgene, and CD16 transgene.

181. A recombinant peptide epitope fusion protein comprising: (1) a hypoimmunity factor selected from the group consisting of CD47, CD24, CD200, HLA-G, HLA-E, HLA-C, HLA-E heavy chain, PD-L1, IDO1, CTLA4-Ig, IL-10, IL-35, FASL, Serpinb9, CCl21, Mfge8, and membrane-bound forms thereof; and (2) a surface-exposed peptide epitope heterologous to the hypoimmunity factor selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

182. A construct encoding a recombinant peptide epitope fusion protein comprising: (1) a sequence encoding a hypoimmunity factor selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof; and (2) a sequence encoding a surface-exposed peptide epitope heterologous to the hypoimmunity factor selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

183. The protein of claim 181 or the construct of claim 182, wherein the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

184. The protein of claim 181 or 183, wherein the hypoimmunity factor and/or the peptide epitope is at the N-terminus of the fusion protein.

185. The protein of any one of claim 181 or 183, further comprising a linker connecting the hypoimmunity factor and the peptide epitope and/or located at the N-terminus or C-terminus of the fusion protein, wherein the linker is selected from any one of the linkers provided in Table 3.

186. The construct of any one of claim 182 or 183, wherein the sequence encoding the hypoimmunity factor is 5′ of the sequence encoding the peptide epitope and/or the sequence encoding the peptide epitope is at the 5′ of the sequence encoding the hypoimmunity factor.

187. The construct of any one of claim 182 or 183, further comprising a sequence encoding a linker connecting the sequence encoding the hypoimmunity factor and the sequence encoding the peptide epitope and/or located at the N-terminus or C-terminus of the fusion protein.

188. The construct of claim 187, wherein the linker is selected from any one of the linkers provided in Table 3.

189. The construct of any one of claim 182-184 or 186-188, further comprising a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

190. The construct of any one of claim 182-184 or 186-189, further comprising a first homology arm and a second homology arm homologous to a target gene locus for CRISPR-based homology directed repair.

191. The construct of any one of claim 182-184 or 186-190, further comprising a vector backbone for lentiviral expression.

192. A method comprising transducing an isolated cell with the construct of claim 191; and selecting the isolated cell that expresses the recombinant peptide epitope fusion protein.

193. An isolated cell or a population thereof comprising a construct of any one of claim 182-184 or 186-191.

194. The isolated cell or the population thereof of claim 193, wherein the isolated cell is an isolated human cell further comprising deletion or reduced expression of MHC class I human leukocyte antigens and/or deletion or reduced expression of MHC class II human leukocyte antigens compared to an unmodified human cell.

195. The isolated cell or the population thereof of any one of claims 193-194, wherein the isolated cell further comprises deletion or reduced expression of CIITA, B2M, and/or NLRC5.

196. The isolated cell or the population thereof of any one of claims 193-195, wherein the isolated cell is hypoimmunogenic and a stem cell.

197. A differentiated cell or a population thereof prepared by culturing the stem cell of claim 196 under differentiation conditions appropriate for differentiation of a stem cell into the cell type selected from the group consisting of cardiac cells, liver cells, kidney cells, pancreatic cells, neural cells, immune cells, mesenchymal cells, and endothelial cells.

198. A method of treating a patient in need of cell therapy comprising administering to patient the differentiated cell or the population thereof of claim 197.

199. A method of treating a patient comprising administering to a patient previously administered the differentiated cell or the population thereof of claim 197 an antibody that binds the peptide epitope.

200. The method of claim 199, wherein the antibody mediates ADCC or CDC.

201. A recombinant CD47-internal-peptide epitope fusion protein comprising from N to C-terminal: (1) a human CD47 fragment comprising a IgV domain of CD47; (2) a first linker; (3) a heterologous peptide epitope; (4) a second linker; and (5) a human CD47 transmembrane domain.

202. The protein of claim 201, wherein the human CD47 fragment comprising the IgV domain comprises amino acid residues 1-127 of the human CD47 protein.

203. The protein of claim 201 or 202, wherein the human CD47 transmembrane domain comprises amino acid residues 128-348 of the human CD47 protein.

204. The protein of any one of claims 201-203, wherein the first and second linkers are selected from any one of the linkers provided in Table 3.

205. The protein of any one of claims 201-204, wherein the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

206. The protein of claim 205, wherein the CD20 epitope is recognized by a therapeutic antibody selected from the group consisting of obinutuzumab, ublituximab, ocaratuzumab, rituximab, rituximab-RLIb and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof, the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof, the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof, the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

207. A construct comprising from 5′ to 3′ end: (1) a transcriptional regulatory element; (2) a sequence encoding a human CD47 fragment comprising a IgV domain of CD47; (3) a first linker; (4) a sequence encoding a peptide epitope; (5) a second linker; and (6) a sequence encoding a human CD47 fragment comprising a transmembrane domain and C-terminus.

208. The construct of 207, wherein the human CD47 fragment comprising the IgV domain encodes amino acid residues 1-127 of the human CD47 protein.

209. The construct of claim 207 or 208, wherein the human CD47 fragment encoding the transmembrane domain and C-terminus comprises amino acid residues 128-348 of the human CD47 protein.

210. The construct of any one of claims 207-209, wherein the first and second linkers are selected from any one of the linkers provided in Table 3.

211. The construct of any one of claims 207-210, wherein the peptide epitope encoded by the sequence of (4) of the construct is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

212. The construct of claim 211, wherein the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

213. The construct of any one of claims 207-212, wherein the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a AGK promoter, and a UBC promoter.

214. The construct of any one of claims 207-213, further comprising a first homology arm and a second homology arm homologous to a target gene locus for CRISPR-based homology directed repair.

215. The construct of any one of claims 207-214, further comprising a vector backbone for lentiviral expression.

216. A method comprising transducing an isolated cell with the construct of claim 215; and selecting the isolated cell that expresses the CD47-internal-peptide epitope fusion protein.

217. An isolated cell or a population thereof comprising a construct of any one of claims 207-215.

218. The isolated cell or the population thereof of claim 217, wherein the isolated cell is an isolated engineered human cell further comprising deletion or reduced expression of MHC class I human leukocyte antigens and/or deletion or reduced expression of MHC class II human leukocyte antigens compared to an unmodified human cell.

219. The isolated cell or the population thereof of any one of claims 217-218, wherein the isolated cell further comprises deletion or reduced expression of CIITA, B2M, and/or NLRC5.

220. The isolated cell or the population thereof of any one of claims 217-219, wherein the isolated cell is hypoimmunogenic and a stem cell.

221. A differentiated cell or a population thereof prepared by culturing the stem cell of claim 220 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

222. A method of treating a patient in need of cell therapy comprising administering to patient the differentiated cell or the population thereof of claim 221.

223. A method of treating a patient previously administered the differentiated cell or the population thereof of claim 221, comprising administering to the patient an antibody that binds the peptide epitope.

224. The method of claim 223, wherein the antibody mediates ADCC or CDC.

225. A construct comprising (1) a transcriptional regulatory element, (2) an essential cell factor gene, (3) a post-transcriptional or post-translational regulatory element, and (4) a polyadenylation sequence.

226. The construct of claim 225, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

227. The construct of claim 225 or 226, wherein the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

228. The construct of any one of claims 225-227, wherein the post-transcriptional regulatory element is a RNA regulation system selected from the group consisting of an inducible shRNA, an inducible siRNA, a CRISPR interference (CRISPRi), and a RNA targeting nuclease system.

229. The construct of any one of claims 225-227, wherein the post-translational regulatory element is an inducible protein degradation system is selected from the group consisting of a small molecule-assisted shutoff (SMASH) system, a shield-1-inducible degron, an auxin-inducible degron, an IMid-inducible degron, a peptidic degron, a proteolysis targeting chimera, and an antibody for targeted degradation.

230. An isolated cell comprising a recombinant essential cell factor under the control of a post-transcriptional or post-translational regulatory element, wherein the endogenous essential cell factor gene is inactivated and expression of the recombinant essential cell factor is controllable by an exogenous factor.

231. The isolated cell of claim 230, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, and a spliceosome subunit protein.

232. The isolated cell of claim 230-231, wherein the post-transcriptional regulatory element is a RNA regulation system selected from the group consisting of an inducible shRNA, an inducible siRNA, a CRISPR interference (CRISPRi), and a RNA targeting nuclease system.

233. The isolated cell of any one of claims 230-232, wherein the post-translational regulatory element is an inducible protein degradation system is selected from the group consisting of a small molecule-assisted shutoff (SMASH) system, a shield-1-inducible degron, an auxin-inducible degron, an IMid-inducible degron, a peptidic degron, a proteolysis targeting chimera, and an antibody for targeted degradation.

234. The isolated cell or the population thereof of any one of claims 230-233, wherein the isolated cell is an autologous human cell or an allogeneic human cell.

235. The isolated cell or the population thereof of claim 234, wherein the isolated cell is an isolated engineered human cell further comprising deletion or reduced expression of MHC class I human leukocyte antigens and/or deletion or reduced expression of MHC class II human leukocyte antigens compared to an unmodified human cell.

236. The isolated cell or the population thereof of claim 234 or 235, wherein the isolated cell further comprises deletion or reduced expression of CIITA, B2M, and/or NLRC5.

237. The isolated cell or the population thereof of any one of claims 234-236, wherein the isolated cell is hypoimmunogenic and selected from the group consisting of a stem cell and a differentiated cell.

238. A bicistronic construct comprising from 5′ to 3′ end: (1) a transcriptional regulatory element; (2) a sequence encoding a surface-exposed peptide epitope: (3) a ribosomal skipping sequence; and (4) a sequence encoding a hypoimmunity factor.

239. The construct of claim 238, wherein the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

240. The construct of claim 238 or 239, wherein the surface-exposed peptide epitope encoded by the sequence of (2) of the construct is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

241. The construct of claim 240, wherein the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

242. The construct of any one of claims 238-241, wherein the ribosomal skipping sequence comprises a sequence encoding an IRES sequence or a sequence encoding a 2A-coding sequence.

243. The construct of any one of claims 238-242, wherein the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

244. The construct of any one of claims 238-243, further comprising a first homology arm and a second homology arm homologous to a target gene locus for CRISPR-based homology directed repair.

245. The construct of any one of claims 238-244, further comprising a vector backbone for lentiviral expression.

246. A method comprising transducing an isolated cell with the construct of claim 245; and selecting the isolated cell that expresses the hypoimmunity factor and the peptide epitope.

247. An isolated cell or a population thereof comprising a construct of any one of claims 238-245.

248. The isolated cell or the population thereof of claim 247, wherein the isolated cell is an isolated human engineered cell further comprising deletion or reduced expression of MHC class I human leukocyte antigens and/or deletion or reduced expression of MHC class II human leukocyte antigens compared to an unmodified human cell.

249. The isolated cell or the population thereof of any one of claims 247-248, wherein the isolated cell further comprises deletion or reduced expression of CIITA, B2M, and/or NLRC5.

250. The isolated cell or the population thereof of any one of claims 247-249, wherein the isolated cell is hypoimmunogenic and a stem cell.

251. A differentiated cell or a population thereof prepared by culturing the stem cell of claim 250 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

252. A method of treating a patient in need of cell therapy comprising administering to patient the differentiated cell or the population thereof of claim 251.

253. A method of treating a patient comprising administering to a patient previously administered the differentiated cell or the population thereof of claim 251 an antibody that binds to the peptide epitope.

254. The method of claim 253, wherein the antibody mediates ADCC or CDC.

255. A pluripotent stem cell comprising (i) reduced or silenced expression of MHC class I molecules and/or MHC class II molecules, (ii) a safety switch transgene and (iii) a hypoimmunity factor gene, wherein expression of the safety switch transgene modulates expression of the hypoimmunity factor gene.

256. A pluripotent stem cell comprising (i) reduced or silenced expression of B2M and CIITA, (ii) overexpression of CD47, (iii) a safety switch transgene, and (iv) a hypoimmunity factor gene, wherein expression of the safety switch transgene modulates expression of the hypoimmunity factor gene.

257. A pluripotent stem cell comprising (i) reduced or silenced expression of MHC class I molecules and/or MHC class II molecules, (ii) a safety switch and (iv) a hypoimmunity factor, wherein expression of the safety switch modulates expression of the hypoimmunity factor.

258. A pluripotent stem cell comprising (i) reduced or silenced expression of B2M and CIITA, (ii) overexpression of CD47, (iii) a safety switch and (iv) a hypoimmunity factor, wherein expression of the safety switch modulates expression of the hypoimmunity factor.

259. A pluripotent stem cell comprising (i) reduced or silenced expression of MHC class I molecules and/or MHC class II molecules, and (ii) a hypoimmunity factor linked to a surface-exposed peptide epitope; wherein the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope, and wherein the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

260. A pluripotent stem cell comprising (i) reduced or silenced expression of B2M and CIITA, (ii) overexpression of CD47, and (iii) a hypoimmunity factor linked to a surface-exposed peptide epitope; wherein the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope, and wherein the hypoimmunity factor is selected from the group consisting of CD47, CD24, CD200, HLA-G, HLA-E, HLA-C, HLA-E heavy chain, PD-L1, ID01, CTLA4-Ig, IL-10, IL-35, FASL, Serpinb9, CCl21, Mfge8, and membrane-bound forms thereof.

261. A construct comprising from 5′ to 3′ end: (1) a safety switch transgene; (2) a ribosomal skipping sequence and/or a sequence encoding a linker; and (3) an essential cell factor gene.

262. A construct comprising from 5′ to 3′ end: (1) an essential cell factor gene: (2) a ribosomal skipping sequence or a linker; and (3) a safety switch transgene.

263. The construct of claim 261 or 262, wherein the safety switch transgene is selected from the group consisting of a HSVtk gene, a cytosine deaminase gene, a nitroreductase gene, a purine nucleoside phosphorylase gene, a horseradish peroxidase gene, iCaspase9 gene, HER1 transgene, RQR8 transgene, CD20 transgene, CCR4 transgene, HER2 transgene, CD19 transgene, MUC1 transgene, EGFR transgene, GD2 transgene, PSMA transgene, CD16 transgene, and CD30 transgene.

264. The construct of any one of claims 261-263, wherein the ribosomal skipping sequence comprises a sequence encoding an IRES sequence or a sequence encoding a 2A-coding sequence.

265. The construct of any one of claims 261-264, wherein the linker is selected from any one of the linkers provided in Table 3.

266. The construct of any one of claims 261-265, wherein the hypoimmunity gene is selected from the group consisting of: CD47, CD24, CD200, HLA-G, HLA-E, HLA-C, HLA-E heavy chain, PD-L1, IDO1, CTLA4-Ig, IL-10, IL-35, FASL, Serpinb9, CCl21, and Mfge8.

267. The construct of any one of claim 261 or 263-266, further comprising a transcriptional regulatory element operably linked to the safety switch transgene and a polyadenylation sequence at the 3′ end of the hypoimmunity gene, or a transcriptional regulatory element operably linked to the hypoimmunity gene and a polyadenylation sequence at the 3′ end of the safety switch transgene.

268. The construct of any one of claims 261-267, wherein the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

269. The construct of any one of claims 261-268, further comprising a vector backbone for lentiviral expression.

270. A method comprising transducing an isolated cell with the construct of claim 269; and selecting the isolated cell carrying the safety switch transgene and the hypoimmunity gene.

271. An isolated cell or a population thereof comprising a construct of any one of claims 261-269.

272. The isolated cell or the population thereof of claim 271, wherein the construct has been introduced into a target gene locus.

273. The isolated cell or the population thereof of claim 271 or 272, wherein the target gene locus is selected from the group consisting of a safe harbor locus selected from the group consisting of an AAVS1 locus, a CLBYL locus, a CXCR4 locus, a Rosa26 locus, and a CCR5 locus and an immune signaling gene locus selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

274. The isolated cell or the population thereof of any one of claims 271-273, wherein the isolated cell further comprises deletion or reduced expression of MHC class I human leukocyte antigens and/or deletion or reduced expression of MHC class II human leukocyte antigens compared to an unmodified human cell.

275. The isolated cell or the population thereof of any one of claims 271-274, wherein the isolated cell further comprises deletion or reduced expression of CIITA, B2M, and/or NLRC5.

276. The isolated cell or the population thereof of any one of claims 271-275, wherein the isolated cell is hypoimmunogenic and a stem cell.

277. A differentiated cell or a population thereof prepared by culturing the stem cell of claim 276 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

278. A method of treating a patient in need of cell therapy comprising administering to patient the differentiated cell or the population thereof of claim 277.

279. A method of treating a patient previously administered the differentiated cell or the population thereof of claim 277, comprising activating a safety switch in the patient.

280. A recombinant peptide epitope fusion protein comprising: (1) an essential cell factor; and (2) a surface-exposed peptide epitope heterologous to the essential cell factor.

281. The protein of claim 280, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, a spliceosome subunit protein, and membrane-bound forms thereof.

282. The protein of claim 280 or 281, wherein the peptide epitope is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

283. The protein of claim 282, wherein the CD20 epitope is recognized by a therapeutic antibody selected from the group consisting of obinutuzumab, ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof the GD2 epitope is recognized by a therapeutic antibody selected from the group consisting of Hu14.18K322A, Hu14.18-1L2, Hu3F8, dinituximab, c.60C3-RLIc, and biosimilars thereof; the PSMA epitope is recognized by a therapeutic antibody selected from the group consisting of KM2812 and biosimilars thereof the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

284. The protein of any one of claims 280-283, wherein the essential cell factor is at the N-terminus of the fusion protein.

285. The protein of any one of claims 280-284, wherein the peptide epitope is at the N-terminus of the fusion protein.

286. The protein of any one of claims 280-285, further comprising a linker connecting the essential cell factor and the peptide epitope.

287. The protein of any one of claims 280-286, further comprising a linker located at the N-terminus of the peptide epitope.

288. The protein of claim 286 or 287, wherein the linker is selected from any one of the linkers provided in Table 3.

289. A construct encoding a recombinant peptide epitope fusion protein comprising: (1) a sequence encoding an essential cell factor; and (2) a sequence encoding a surface-exposed peptide epitope heterologous to the essential cell factor.

290. The construct of claim 289, wherein the essential cell factor is selected from the group consisting of RpS2, RpS9, RpS11, RpS13, RpS18, RpL8, RpL11, RpL32, RpL36, Rpn22, Psmd14, PSMA3, a ribosome subunit protein, a proteasome subunit protein, a spliceosome subunit protein, and membrane-bound forms thereof.

291. The construct of claim 289 or 290, wherein the peptide epitope encoded by the sequence of (2) of the construct is selected from the group consisting of a CD20 epitope, CCR4 epitope, HER2 epitope, CD19 epitope, MUC1 epitope, EGFR epitope, GD2 epitope, PSMA epitope, CD16 epitope, and CD30 epitope.

292. The construct of claim 291, wherein the CD20 epitope is recognized by a therapeutic antibody selected from the group consisting of obinutuzumab; ublituximab, ocaratuzumab, rituximab, rituximab-RLIb, and biosimilars thereof; the CCR4 epitope is recognized by a therapeutic antibody selected from the group consisting of mogamulizumab and biosimilars thereof; the HER2 epitope is recognized by a therapeutic antibody selected from the group consisting of margetuximab, trastuzumab, TrasGEX, and biosimilars thereof; the CD19 epitope is recognized by a therapeutic antibody selected from the group consisting of MOR208 and biosimilars thereof; the MUC1 epitope is recognized by a therapeutic antibody selected from the group consisting of gatipotuzumab and biosimilars thereof; the EGFR epitope is recognized by a therapeutic antibody selected from the group consisting of tomuzotuximab, RO5083945 (GA201), cetuximab, and biosimilars thereof; the GD2 epitope is recognized by a therapeutic antibody selected from the group consisting of Hu14.18K322A, Hu14.18-IL2, Hu3F8, dinituximab, c.60C3-RLIc, and biosimilars thereof; the PSMA epitope is recognized by a therapeutic antibody selected from the group consisting of KM2812 and biosimilars thereof; the CD30 or CD16 epitope is recognized by a therapeutic antibody selected from the group consisting of AFM13 and biosimilars thereof, or the CD20 or CD16 epitope is recognized by a therapeutic antibody selected from the group consisting of (CD20)2×CD16 and biosimilars thereof.

293. The construct of any one of claims 289-292, wherein the sequence encoding the essential cell factor is 5′ of the sequence encoding the peptide epitope.

294. The construct of any one of claims 289-293, wherein the sequence encoding the peptide epitope is at the 5′ of the sequence encoding the essential cell factor.

295. The construct of any one of claims 289-294, further comprising a sequence encoding a linker connecting the sequence encoding the essential cell factor and the sequence encoding the peptide epitope.

296. The construct of any one of claims 289-295, further comprising a sequence encoding a linker located at the N-terminus or C-terminus of the fusion protein.

297. The construct of any one of claims 295-296, wherein the linker is selected from any one of the linkers provided in Table 3.

298. The construct of any one of claims 289-297, further comprising a transcriptional regulatory element selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

299. The construct of any one of claims 289-298, further comprising a first homology arm and a second homology arm homologous to a target gene locus for CRISPR-based homology directed repair.

300. The construct of any one of claims 289-299, further comprising a vector backbone for lentiviral expression.

301. A method comprising transducing an isolated cell with the construct of claim 300; and selecting the isolated cell expressing the recombinant peptide epitope fusion protein.

302. An isolated cell or a population thereof comprising a construct of any one of claims 289-300.

303. The isolated cell or the population thereof of claim 302, wherein the isolated cell is an isolated human cell further comprising deletion or reduced expression of MHC class I human leukocyte antigens and/or deletion or reduced expression of MHC class II human leukocyte antigens compared to an unmodified human cell.

304. The isolated cell or the population thereof of any one of claims 302-303, wherein the isolated cell further comprises deletion or reduced expression of CIITA, B2M, and/or NLRC5.

305. The isolated cell or the population thereof of any one of claims 302-304, wherein the isolated cell is hypoimmunogenic and a stem cell.

306. A differentiated cell or a population thereof prepared by culturing the stem cell of claim 305 under differentiation conditions appropriate for differentiation of the stem cell into a cell type selected from the group consisting of cardiac cells, liver cells, kidney cells, pancreatic cells, neural cells, immune cells, mesenchymal cells, and endothelial cells.

307. A method of treating a patient in need of cell therapy comprising administering to patient the differentiated cell or the population thereof of claim 306.

308. A method of treating a patient comprising administering to a patient previously administered the differentiated cell or the population thereof of claim 307 an antibody that binds the peptide epitope.

309. The method of claim 308, wherein the antibody mediates ADCC or CDC.

310. A construct for homology directed repair into a safe harbor locus comprising from 5′ to 3′ end: (1) a first homology arm homologous to a first endogenous sequence of a safe harbor locus; (2) a transcriptional regulatory element; (3) an HSVtk safety switch transgene; (4) a ribosomal skipping sequence and/or a sequence encoding a linker; (5) a CD47 hypoimmunity gene; (6) a polyadenylation sequence; and (7) a second homology arm homologous to a second endogenous sequence of the safe harbor locus.

311. A construct for homology directed repair into a safe harbor locus comprising from 5′ to 3′ end: (1) a first homology arm homologous to a first endogenous sequence of an immune signaling gene locus; (2) a transcriptional regulatory element; (3) an HSVtk safety switch transgene; (4) a ribosomal skipping sequence and/or a sequence encoding a linker; (5) an CD47 hypoimmunity gene; (6) a polyadenylation sequence; and (7) a second homology arm homologous to a second endogenous sequence of the immune signaling gene locus.

312. The construct of claim 310 or 311, wherein the transcriptional regulatory element is selected from the group consisting of an EF1A promoter, an EFS promoter, a CMV promoter, a CAGGS promoter, an SV40 promoter, a COPIA promoter, an ACT5C promoter, a TRE promoter, a CBh promoter, a PGK promoter, and a UBC promoter.

313. The construct of any one of claims 310-312, further comprising a vector backbone for lentiviral expression.

314. An isolated cell or a population thereof comprising a safety switch transgene and a hypoimmunity gene integrated into a safe harbor locus or an immune signaling gene locus, wherein the construct of any one of claims 310-313 has recombined into the endogenous safe harbor locus of the isolated cell or into the endogenous targeted gene locus of the isolated cell.

315. The isolated cell or the population thereof of claim 314, wherein the isolated cell further comprises deletion or reduced expression of MHC class I human leukocyte antigens and/or deletion or reduced expression of MHC class II human leukocyte antigens compared to an unmodified human cell.

316. The isolated cell or the population thereof of claim 314 or 315, wherein the isolated cell further comprises deletion or reduced expression of CIITA, B2M, and/or NLRC5.

317. The isolated cell or the population thereof of any one of claims 314-316, wherein the isolated cell is hypoimmunogenic and a stem cell.

318. A differentiated cell or a population thereof prepared by culturing the stem cell of claim 317 under differentiation conditions appropriate for differentiation into pancreatic cells.

319. The differentiated cell or the population thereof of claim 318, wherein the pancreatic cells are beta-islet cells.

320. A method of treating a patient in need of cell therapy comprising administering to a patient the differentiated cell or the population thereof of claim 318 or 319, and activating the safety switch in a patient previously administered the differentiated cell or the population thereof of claim 318 or 319.


SEQUENCE LISTING (110 sequences)

SEQ ID NO: 1 - protein, 4 aa, Artificial Sequence
  Linker
  REPEAT (1)...(4): n is 1-10
GGGS

SEQ ID NO: 2 - protein, 8 aa, Artificial Sequence
  Linker
  REPEAT (1)...(8): n is 1-10
GGGSGGGS

SEQ ID NO: 3 - protein, 3 aa, Artificial Sequence
  Linker
  REPEAT (1)...(3): n is 1-10
GSG

SEQ ID NO: 4 - protein, 21 aa, Artificial Sequence
  T2A self-cleaving peptide sequence
GSGEGRGSLLTCGDVEENPGP

SEQ ID NO: 5 - protein, 22 aa, Artificial Sequence
  P2A self-cleaving peptide sequence
GSGATNFSLLKQAGDVEENPGP

SEQ ID NO: 6 - protein, 23 aa, Artificial Sequence
  E2A self-cleaving peptide sequence
GSGQCTNYALLKLAGDVESNPGP

SEQ ID NO: 7 - protein, 25 aa, Artificial Sequence
  F2A self-cleaving peptide sequence
GSGVKQTLNFDLLKLAGDVESNPGP

SEQ ID NO: 8 - protein, 5 aa, Artificial Sequence
  Linker RH_1
EAAAK

SEQ ID NO: 9 - protein, 10 aa, Artificial Sequence
  Linker RH_2
EAAAKEAAAK

SEQ ID NO: 10 - protein, 15 aa, Artificial Sequence
  Linker RH_3
EAAAKEAAAKEAAAK

SEQ ID NO: 11 - protein, 17 aa, Artificial Sequence
  Linker RH_5
AEAAAKEAAAKEAAAKA

SEQ ID NO: 12 - protein, 24 aa, Artificial Sequence
  Linker RH_6
AEAAAKAEAAAKAEAAAKAEAAAK

SEQ ID NO: 13 - protein, 30 aa, Artificial Sequence
  Linker RH_7
AEAAAKAEAAAKAEAAAKAEAAAKAEAAAK

SEQ ID NO: 14 - protein, 46 aa, Artificial Sequence
  Linker RH_8
AEAAAKEAAAKEAAAKEAAAKALEAEAAAKEAAAKEAAAKEAAAKA

SEQ ID NO: 15 - protein, 5 aa, Artificial Sequence
  Linker REPR_15
PAPAP

SEQ ID NO: 16 - protein, 10 aa, Artificial Sequence
  Linker REPR_2
APAPAPAPAP

SEQ ID NO: 17 - protein, 16 aa, Artificial Sequence
  Linker REPR_5
APAPAPAPAPAPAPAP

SEQ ID NO: 18 - protein, 22 aa, Artificial Sequence
  Linker REPR_8
APAPAPAPAPAPAPAPAPAPAP

SEQ ID NO: 19 - protein, 28 aa, Artificial Sequence
  Linker REPR_11
APAPAPAPAPAPAPAPAPAPAPAPAPAP

SEQ ID NO: 20 - protein, 34 aa, Artificial Sequence
  Linker REPR_14
APAPAPAPAPAPAPAPAPAPAPAPAPAPAPAPAP

SEQ ID NO: 21 - protein, 18 aa, Artificial Sequence
  Linker FGSR_15
KESGSVSSEQLAQFRSLD

SEQ ID NO: 22 - protein, 14 aa, Artificial Sequence
  Linker FGSR_16
EGKSSGSGSESKST

SEQ ID NO: 23 - protein, 21 aa, Artificial Sequence
  Linker FGSR_18
NSGAGGSGGSSGSDGASGSRD

SEQ ID NO: 24 - protein, 5 aa, Artificial Sequence
  Linker FGSR_1
GGGGS

SEQ ID NO: 25 - protein, 4 aa, Artificial Sequence
  Linker FGSR_10
GGGS

SEQ ID NO: 26 - protein, 2 aa, Artificial Sequence
  Linker FGSR_14
GS

SEQ ID NO: 27 - protein, 12 aa, Artificial Sequence
  Linker FGSR_17
GSAGSAAGSGEF

SEQ ID NO: 28 - protein, 10 aa, Artificial Sequence
  Linker FGSR_2
GGGGSGGGGS

SEQ ID NO: 29 - protein, 15 aa, Artificial Sequence
  Linker FGSR_3
GGGGSGGGGSGGGGS

SEQ ID NO: 30 - protein, 20 aa, Artificial Sequence
  Linker FGSR_4
GGGGSGGGGSGGGGSGGGGS

SEQ ID NO: 31 - protein, 3 aa, Artificial Sequence
  Linker FGSR_5
GGS

SEQ ID NO: 32 - protein, 6 aa, Artificial Sequence
  Linker FGSR_6
GGGGGG

SEQ ID NO: 33 - protein, 8 aa, Artificial Sequence
  Linker FGSR_7
GGGGGGGG

SEQ ID NO: 34 - protein, 5 aa, Artificial Sequence
  Linker AP_5
SSSSG

SEQ ID NO: 35 - protein, 10 aa, Artificial Sequence
  Linker AP_6
SSSSGSSSSG

SEQ ID NO: 36 - protein, 15 aa, Artificial Sequence
  Linker AP_7
SSSSGSSSSGSSSSG

SEQ ID NO: 37 - protein, 20 aa, Artificial Sequence
  Linker AP_8
SSSSGSSSSGSSSSGSSSSG

SEQ ID NO: 38 - protein, 25 aa, Artificial Sequence
  Linker AP_9
SSSSGSSSSGSSSSGSSSSGSSSSG

SEQ ID NO: 39 - protein, 62 aa, Artificial Sequence
  Linker AP_18
ALSNSIMYFSHFVPVFLPAKPTTTPAPRPPTPAPTIASQPLSLRPEASRPAAGGAVHTRGLD

SEQ ID NO: 40 - protein, 5 aa, Artificial Sequence
  Linker NP_35
TVAAP

SEQ ID NO: 41 - protein, 6 aa, Artificial Sequence
  Linker NP_36
ASTKGP

SEQ ID NO: 42 - protein, 13 aa, Artificial Sequence
  Linker NP_37
QPKAAPSVTLFPP

SEQ ID NO: 43 - protein, 12 aa, Artificial Sequence
  Linker NP_38
TVAAPSVFIFPP

SEQ ID NO: 44 - protein, 13 aa, Artificial Sequence
  Linker NP_39
ASTKGPSVFPLAP

SEQ ID NO: 45 - protein, 22 aa, Artificial Sequence
  Linker NP_1
AQGTLSPADKTNVKAAWGKVMT

SEQ ID NO: 46 - protein, 36 aa, Artificial Sequence
  Linker NP_21
PGNPTTTVVPPASTSTSRPTSSTSSPVSTPTGQPGG

SEQ ID NO: 47 - protein, 19 aa, Artificial Sequence
  Linker 1EI1A
GGKFDDNSYKVSGGLHGVG

SEQ ID NO: 48 - protein, 44 aa, Artificial Sequence
  Linker 1KQFA
GRLFAINKMAEGPFPEHYEPIETPLGTNPLHPNVVSNPVVRLYE

SEQ ID NO: 49 - protein, 51 aa, Artificial Sequence
  Linker 1B8TA
YGPKGKGKGMGAGTLSTDKGESLGIKYEEGQSHRPTNPNASRMAQKVGGSD

SEQ ID NO: 50 - protein, 5 aa, Artificial Sequence
  Linker JP1
GKGKG

SEQ ID NO: 51 - protein, 9 aa, Artificial Sequence
  Linker JP2
GKGKGMGAG

SEQ ID NO: 52 - protein, 16 aa, Artificial Sequence
  Linker JP3
GKGKGMGAGTLSTDKG

SEQ ID NO: 53 - protein, 20 aa, Artificial Sequence
  Linker JP4
GKGKGMGAGTLSTDKGESLG

SEQ ID NO: 54 - protein, 85 aa, Artificial Sequence
  Linker 2ZT5A
KPLKEPKTVNVVQFEPSKGAIGKAYKKDAKLVMEYLAICDECYITEMEMLLNEKGEFTIETEGKTFQLTKDMINVKRFQKTLYVE

SEQ ID NO: 55 - protein, 6 aa, Artificial Sequence
  Linker LP1
IEGRMD

SEQ ID NO: 56 - protein, 20 aa, Artificial Sequence
  Linker pVT197
GGGGSGGGGSGGGGSAAQPA

SEQ ID NO: 57 - protein, 21 aa, Artificial Sequence
  Linker pVT311
GGGGSGGGGSGGGGSAAQPAS

SEQ ID NO: 58 - protein, 16 aa, Artificial Sequence
  Linker LP1
GGGGSLVPRGSGGGGS

SEQ ID NO: 59 - protein, 15 aa, Artificial Sequence
  Linker LP2
GGGGSLVPRGSGGGG

SEQ ID NO: 60 - protein, 11 aa, Artificial Sequence
  Linker LP3
GGSGGHMGSGG

SEQ ID NO: 61 - protein, 11 aa, Artificial Sequence
  Linker LP4
GGSGGSGGSGG

SEQ ID NO: 62 - protein, 5 aa, Artificial Sequence
  Linker LP5
GGSGG

SEQ ID NO: 63 - protein, 8 aa, Artificial Sequence
  Linker LP6
GGSGGGGG

SEQ ID NO: 64 - protein, 18 aa, Artificial Sequence
  Linker LP7
GGGSEGGGSEGGGSEGGG

SEQ ID NO: 65 - protein, 11 aa, Artificial Sequence
  Linker LP8
GSGGGTGGGSG

SEQ ID NO: 66 - protein, 2 aa, Artificial Sequence
  Linker LP9
GT

SEQ ID NO: 67 - protein, 6 aa, Artificial Sequence
  Linker LP10
GSGSGS

SEQ ID NO: 68 - protein, 8 aa, Artificial Sequence
  Linker LP11
GSGSGSGS

SEQ ID NO: 69 - protein, 10 aa, Artificial Sequence
  Linker LP12
GSGSGSGSGS

SEQ ID NO: 70 - protein, 12 aa, Artificial Sequence
  Linker LP13
GSGSGSGSGSGS

SEQ ID NO: 71 - protein, 14 aa, Artificial Sequence
  Linker LP14
GSGSGSGSGSGSGS

SEQ ID NO: 72 - protein, 16 aa, Artificial Sequence
  Linker LP15
GSGSGSGSGSGSGSGS

SEQ ID NO: 73 - protein, 10 aa, Artificial Sequence
  Linker LP16
SGGSGGSSHS

SEQ ID NO: 74 - protein, 8 aa, Artificial Sequence
  Linker LP17
AAGAATAA

SEQ ID NO: 75 - protein, 15 aa, Artificial Sequence
  Linker LP18
EPKSADKTHTAPPAP

SEQ ID NO: 76 - protein, 12 aa, Artificial Sequence
  Linker JP5
EPKSDKTHTPPP

SEQ ID NO: 77 - protein, 15 aa, Artificial Sequence
  Linker JP6
EPKSCDKTHTCPPCP

SEQ ID NO: 78 - protein, 23 aa, Artificial Sequence
  Linker JP7
EPKSCDKTHTCPPCPAPELLGGP

SEQ ID NO: 79 - protein, 20 aa, Artificial Sequence
  Linker JP8
EPKSDKTHTPPPAPELLGGP

SEQ ID NO: 80 - protein, 21 aa, Artificial Sequence
  Linker JP9
EPKSCDKTHTCPPCPAPEGGP

SEQ ID NO: 81 - protein, 18 aa, Artificial Sequence
  Linker JP10
EPKSDKTHTPPPAPEGGP

SEQ ID NO: 82 - protein, 8 aa, Artificial Sequence
  Linker JP11
ERKVEPPP

SEQ ID NO: 83 - protein, 12 aa, Artificial Sequence
  Linker JP12
ERKCCVECPPCP

SEQ ID NO: 84 - protein, 19 aa, Artificial Sequence
  Linker JP13
ERKCCVECPPCPAPPVAGP

SEQ ID NO: 85 - protein, 15 aa, Artificial Sequence
  Linker JP14
ERKVEPPPAPPVAGP

SEQ ID NO: 86 - protein, 59 aa, Artificial Sequence
  Linker JP15
ELKTPLGDTTHTCPRCPEPKSCDTPPPCPRCPEPKSCDTPPPCPRCPEPKSCDTPPPCP

SEQ ID NO: 87 - protein, 49 aa, Artificial Sequence
  Linker JP16
ELKTPLGDTTHTPRPEPKSDTPPPPRPEPKSDTPPPPRPEPKSDTPPPP

SEQ ID NO: 88 - protein, 12 aa, Artificial Sequence
  Linker NP_8
ELKTPLGDTTHT

SEQ ID NO: 89 - protein, 10 aa, Artificial Sequence
  Linker JP17
ESKYGPPPSP

SEQ ID NO: 90 - protein, 12 aa, Artificial Sequence
  Linker JP18
ESKYGPPCPSCP

SEQ ID NO: 91 - protein, 20 aa, Artificial Sequence
  Linker JP19
ESKYGPPCPSCPAPEFLGGP

SEQ ID NO: 92 - protein, 18 aa, Artificial Sequence
  Linker JP20
ESKYGPPPSPAPEFLGGP

SEQ ID NO: 93 - protein, 18 aa, Artificial Sequence
  Linker JP21
ESKYGPPCPSCPAPEGGP

SEQ ID NO: 94 - protein, 16 aa, Artificial Sequence
  Linker JP22
ESKYGPPPSPAPEGGP

SEQ ID NO: 95 - protein, 21 aa, Artificial Sequence
  Linker JP23
SGSETPGTSESATPEGGSGGS

SEQ ID NO: 96 - protein, 16 aa, Artificial Sequence
  Linker JP24
SGSETPGTSESATPES

SEQ ID NO: 97 - protein, 11 aa, Artificial Sequence
  Linker JP25
SGSETPGTSES

SEQ ID NO: 98 - DNA, 966 bp, Artificial Sequence
  Promoter EF1A
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttc

SEQ ID NO: 99 - DNA, 1177 bp, Artificial Sequence
  Promoter UBC
ggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 100 - DNA, 511 bp, Artificial Sequence
  Promoter Pgk
ttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 101 - DNA, 589 bp, Artificial Sequence
  Promoter CMV
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 102 - DNA, 1718 bp, Artificial Sequence
  Promoter CAGGS
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 103 - DNA, 344 bp, Artificial Sequence
  Promoter SV40
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 104 - DNA, 283 bp, Artificial Sequence
  Promoter COPIA
atctgttggaatatactattcaacctacaaaaataacgttaaacaacactactttatatttgatatgaatggccacaccttttatgccataaaacatattgtaagagaataccactctttttattccttctttccttcttgtacgttttttgctgtgagtaggtcgtggtgctggtgttgcagttgaaataacttaaaatataaatcataaaactcaaacataaacttgactatttatttatttattaagaaaggaaatataaattataaattacaacaggtt

SEQ ID NO: 105 - DNA, 307 bp, Artificial Sequence
  Promoter ACT5C
ggaagtacactcttcatggcgatatacaagacacacacaagcacgaacacccagttgcggaggaaattctccgtaaatgaaaacccaatcggcgaacaattcatacccatatatggtaaaagttttgaacgcgacttgagagcggagagcattgcggctgataaggttttagcgctaagcgggctttataaaacgggctgcgggaccagttttcatatcactaccgtttgagttcttgtgctgtgtggatactcctcccgacacaaagccgctccatcagccagcagtcgtctaatccagagacacc

SEQ ID NO: 106 - DNA, 424 bp, Artificial Sequence
  Promoter TRE
ggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattg

SEQ ID NO: 107 - DNA, 23 bp, Artificial Sequence
  Cas9 Target site
cgtgagtaaacctgaatctttgg

SEQ ID NO: 108 - protein, 10 aa, Artificial Sequence
  CD20 fragment
CPYSNPSLCS

SEQ ID NO: 109 - DNA, 23 bp, Artificial Sequence
  Cas9 Target site
ggggccactagggacaggattgg

SEQ ID NO: 110 - DNA, 23 bp, Artificial Sequence
  Cas9 Target site
ccagctgtggctacaacataggg